SEQUENCE LISTING (34 sequences)

SEQ ID NO: 1 - protein, 279 aa, Escherichia coli
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 2 - protein, 279 aa, Escherichia coli
FACKTANGTAIPIGGGSANVYVNLAPAVNVGQNLVVDLSTQIFCHNDYPETITDYVTLQRGAAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 3 - protein, 160 aa, Escherichia coli
FSCNVDGGSSIGAGTTSVYVNLDPVIQPGQNLVVDLSQHISCWNDYGGWYDTDHINLVQGSAFAGSLQSYKGSLYWNNVTYPFPLTTNTNVLDIGDKTPMPLPLKLYITPVGAAGGVVIKAGEVIARIHMYKIATLGSGNPRNFTWNIISNNSVVMPTGG

SEQ ID NO: 4 - protein, 160 aa, Escherichia coli
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 5 - protein, 160 aa, Klebsiella pneumoniae
FACKTATGATIPIGGGSANVYVNLTPAVNVGQNLVVDLSTQIFCHNDYPETITDYVTLQRGSAYGGVLSSFSGTVKYNGTSYPFPTTTETARVIYDSRTDKPWPAVLYLTPVSTAGGVAITAGSLIAVLILHQTNNYNSDSFQFIWNIYANNDVVVPTGG

SEQ ID NO: 6 - protein, 160 aa, Kluyvera cryocrescens
FACKTASGATIPIGGGSANVYVDLAPAVSVGQNLVVDLSTQIFCHNDYPDTITDYVTLQRGSAYGGILASFSGTVKYNGVSYPFPTTTETARLTYNSKTDKPWPTVLYLTPISSAGGVAITAGTLIAVLILRQTNNKDADDFQFVWNIYANNNVVVPTGG

SEQ ID NO: 7 - protein, 160 aa, Klebsiella sp.
FACRTAAGVTIPIGGGSANVYVNMAPSVSVGQNLVVDLSTQIFCHNDFPDTITDYVTLQRGSAYGGVLKNFTGTVRYNGLAYPFPTTSETARVIYNSKTDKPWPAVLYLTPVSTAGGVAISAGSLVAVLILHQTNSKDADDFQFIWNIYASNDVVVPTGG

SEQ ID NO: 8 - protein, 160 aa, Erwinia tasmaniensis
FACRTAGGVEIPIGGGSANVYVNLTPSVGIGQNLVVDLATQIACRNDYPSTHTDYVSLLQGSAYGGALKNFKGSIIYSDSTYPFPTTSETKILTYKSKTETPWPTKLYLTAIGSAADGVAIKAGTLVAILNMHQTNNYNESNSYVWNVYAQNSVVVPTGG

SEQ ID NO: 9 - protein, 159 aa, Serratia fonticola
FACRTAAGATIPIGGGSANVFVDLTPQIGVGQNLIVDLSRQISCRNDYPDTRIDYVSLQRGSAYGGVLASFTGSFVYDGTSYPFPTTSETKSVVYSTTTMTPWPAVLYLTPISTAGGVAITSGSLIAVLNMHQTNNVGESHAYIWNIYASNSVVIPTGG

SEQ ID NO: 10 - protein, 160 aa, Izhakiella capsodis
FACRTAAGAEIPIGGGSADVYVTLAPSVGVGQNLVVDLSTQISCRNDYPDTYIDYVSLMSGSAYGGALENFKGSIAYSDRTYPFPTTSETIKLTYRSRTLTPWPTKLYLTALGNAGSGVAIQGGSLIAKLNMSQTNNYGDFNTYVWYIYALNSVVVPTGG

SEQ ID NO: 11 - protein, 160 aa, bacteria symbiont BFo2 of Frankliniella occidentalis
FACKTAAGATIPIGGGNANVYVNLTPQVQSGGNLVVDLANSIFCHNDYPASITDYVTLASGSSFNGVLKNFKGSVIYNGQNYSFPTTRETSRVLYRSKSDTAWPARLLLTPISTAEGVVVNSGSLIASLVLHQTNNVGNDSYYYTWNIYASNNVVVPTGG

SEQ ID NO: 12 - protein, 159 aa, Tatumella ptyseos
FTCKTAAGVTVPAGGGDANVYVTLTPSVGASSNLVVDLSQSITCNDEYPPTYIDYVSLQSGSAFGGLLSSFSGTVDYNGTSYAFPTTSETAKVTYNSKSFKSWPVKLNLTPISTAGGVVISSGSLIASLVLHQTNNFSSSASYVWNIYANNNVTIPTGG

SEQ ID NO: 13 - protein, 159 aa, Chania multitudinisentens
FACQTAAGATIPIGGGNADVYVTLSPQIGVGQNLIVDLSTQISCRNDFPQTIKDFVSLQAGSAYGGVLANFRGSFVYGGISYPFPTTNETHSIEFKTTTMTPWPAVLYLTPISSAGGVAITSGSLIAMLNMHQTNDKGESHPYIWRIYANNNVVIPTGG

SEQ ID NO: 14 - protein, 168 aa, Chania multitudinisentens
FTCYDSTGNTLNSASGTATATVYVNLQPSITASQNLVVDLSNSIFCKNDNPTVRNDHVSLLNGSAYGGVLSNFNGTVEYYGSSYPFPLRSATSSKNFTSGTYTKWDAKLYLTPLSSAASGVVVKKNSQFASLVMFQRGSNIVGGGNVHTATFTWNLYANNDVVVPTGG

SEQ ID NO: 15 - protein, 167 aa, Providencia heimbachae
FTCYDSTGNVLNSASGTATANVYVNLQPSVQAGQNLVVDLSQSIYCKNDSPAIRKDLVSMIRGSAYGGVLSKFTGSLRYYGSSYSFPLTSPTHQQNFSSGSYVPWNTQLYLTPISAAGGVVINRGTLFASLVMYQIGSDIPGGGNIHTATFTWNLYANNNVIVPIGG

SEQ ID NO: 16 - protein, 162 aa, Pluralibacter gergoviae
FTCKILSTGQMVQTGSANLYVNLTPQIGVGQNLIVDLTQEIKCKNDSAPPDIDVDHLKLLSGSAYQGALDNFSGAVYWVGKYYPIPLTANTPYYSISNTSYAPLPLKLYLTPLGAAGGKLIRSGELIAVLSVYKIATFDGGYPTTFTWNIYANNDVVLPTGG

SEQ ID NO: 17 - protein, 159 aa, Citrobacter koseri
FTCKVNDTGQTMTSGSANVYVNLTPSVGVGQNLVVDLSSSVSCKNDSSGGSIIDYINLTSGSAFGGALAAFTGSVYWAGNTYPLPMNGNSSVYTITHTDYRGLPLRMYLTATGAAGGVVINSGELIAQLNMHKVASDGNPNNFIWNIYANNNVVVPTGG

SEQ ID NO: 18 - protein, 165 aa, Proteus mirabilis
FTCKVASTGQIVGDGAANIRVNLTPRVEVNQNLVVDLSQQILCKNDSYGGPRDPIDVDHVNLVSGTTFGGALNNFDGSLLWYNSYYPIPLYYDTKVINIKDRNFIPLNLRLYLKSIGAAGGKQINKGDLIAKIEMYKIADWDGGHPRFFTWNIYALNDVIMPTGG

SEQ ID NO: 19 - protein, 158 aa, Tatumella morbirosei
FTCQANGTSISGSGTVTVPVTLAPSVQSTENLVVNLGNSIQCKNDLPSVYKDPIRVGTASAYAGALSAFTGSITYNGDTYSFPLPSPTSWVPTPNGTYAPWNTILYLTPTGSASGVVIQAGQIFATLQLQKEGNPGQVSQTITWNLKANNTVTVPTGG

SEQ ID NO: 20 - protein, 160 aa, Escherichia coli
FSCNVDGGSSIGAGTTSVYVNLDPVIQPGQNLVVDLSQHISCWNDYGGWYDTDHINLVQGSAFAGSLQSYKGSLYWNNVTYPFPLTTNTNVLDIGDKTPMPLPLKLYITPVGAAGGVVIKAGEVIARIHMYKIATLGSGNPRNFTWNIISNNNVVMPTGG

SEQ ID NO: 21 - protein, 159 aa, Tatumella ptyseos
FTCQANGTTINGSGTVTVPVTLTPSLGTTQNLVINLGSSIQCKNDAPKQYTDPIRVGTASAYAGALSSFTGSITYNNSTYAFPLSSPTPYVPTTSGSYVAWNTILYLSPTGAASGVVIQAGQIFASLSLQKENTSTGGVSQTIIWNLKANNTVTVPTGG

SEQ ID NO: 22 - protein, 162 aa, Proteus mirabilis
FGCSVDNGYDIESGNYNIYVQLEAEVQPYQNLIVDLSRHLKCWNSYGGWQDVDHINLEPGTGLSGPLFNFSGSVYWYGNTYSLPLNSATKVLHIGDLTPMPMPMMLYLTPINNSPAGGVLIRSGETIATINMYKIADWDGGNPKYFTWEIVSKYDVTMPISG

SEQ ID NO: 23 - protein, 168 aa, Zymobacter palmae
YTCRYRSSAPAGLAGTTIQGSSNATIPVPVQDTAETGQVNIIDLAAYIECKNDNPGTYTDHMDMQRASTVLSNNFDVRVKANGVEYTVPFSGSATVLHLPKGGSGSYAPIPLQIYYYMKEIPGERVAIKKGQVIGSIQAHKYSIPAGGAPTFTWTFTAANDTIVTSGG

SEQ ID NO: 24 - protein, 168 aa, Zymobacter palmae
YTCRYRSTAPNGLALQVIKGNSNATIQVPVSNTAETGQTNIIDLSSYIECKNDIPQSYSDYMDLQQATTILSNNFDVKVKARSNEYSVPFSGNANILTLPRGGSGSYAPIPLQIYYYMKEIPGEKVAIKKGQTIGAIRAHHHSIPVEGDYIFTWNFVAANETIVTSGG

SEQ ID NO: 25 - protein, 163 aa, Candidatus Erwinia dacicola
FECIDKRDGKPFDAVGQRNIDVTVNLSPEIIIGDVVVFDLSDTFTCRNTMPKTYVDYMLLNSETYQTSLDESFSSGLEVNGERYLNPINSAITVFKLRDGNWHDLQVKAFYQLMNSPGRGVFIKAGTLVASMQMYKWSAPAGGVFTANWRILAANDAHYTSGT

SEQ ID NO: 26 - protein, 163 aa, bacteria symbiont BFo1 of Frankliniella occidentalis
FACIDKRDGKPFNAVGQRNIDITVNLSPEIIIGDVVVFDLANYFTCKNELPNSYVDYMRLNSGTYQTSLDESFSSGLEVNGTRYLNPINSPITVFELRDGNWHDLQVKAFYQLKNSPGRGVFIKAGTVVASMQMYKWSAPAGGVFTANWRILAANDAYYTAGT

SEQ ID NO: 27 - protein, 170 aa, Providencia sneebia
FVCKTNAGQEVSSGTQSINNIPIDNDIFAVPNRINEFANVNDYMTCRNELPSAYVDYLNLQSMTLGPVLQSNPDLKAGVSVRGVRYLAPFSGRDIQIFRLTQSSESLQIKLYIQVNHKPTPSVLIKKGDLLMTLGLQKYATRVRSNNPIDFLNFTWKFYAGNDVIIGTGT

SEQ ID NO: 28 - protein, 163 aa, Erwinia iniecta
FSCIDKRDGKAFDAVGQRNIDVTVNLSPEIMIGDVVIFDLADYFTCMNTIPNRYVDYMRLNAGSYNTTLDASFNSGLEIKGQRYLNPINSSVNVFELRDGNWHDLEVKAFYQLKNSPAKGVYIKAGAVVASMQFYKWSVPAGGVFTANWRIIAANDAYYTSGT

SEQ ID NO: 29 - protein, 165 aa, Erwinia tasmaniensis
YTCRNKQTGQSLKGGNSSVTVPISRTLTFGEQVVFDIGQYYECKNDNPGTYSDYMETQANASSTTLPTAFDTGALINGGKYDFPLPQVNVFTLPKGGDSNYHNVPIQVYYSMKDTPGQLVKISKGQTLATLQLHKYATTTGGPQDPQDFTWNIIAANDSIFTSGS

SEQ ID NO: 30 - protein, 165 aa, Serratia odorifera
FTCRTSDGGLIPPGGSTTPVDVRVRIGPQLSYGKNEIVNVSQVTCKNDVSSWTDYLKTDSPALSMNSAIFGGIGSGMTINGRDYPSPVSSGISVVTLTNLNSQSIAIRVYIVLNRFPTPDIKINKGDVIGQINFSQTNDRPNCPQCGPYRWRLIADNDAYFVTTT

SEQ ID NO: 31 - protein, 167 aa, Erwinia sp.
YICRNKQTGQQLRGGDSPVVIPLSRQITPGEVIFIDLWKYYECRNEEPYFYDDFMYLEDNGISTVLNKDFEVGAYINGGKYDIPVPYTQIFKLPWHGDGNWHAIPFRVFYKVSETPGYLTRISKGQEIATVRLYKYAHFKGGTESDHPRHFIWKIIAGNDSIFTSGT

SEQ ID NO: 32 - protein, 168 aa, Orbus hercynius
FVCYDLSTGKEIKSNGTVSVPIYINSNIVRGENIFGNVGDYLACQNQQPYFYVDHMELKANGITPGAAISHLDNGVYINGARILSTNTPKTNIFSIADNNIYPLDINMFFVVPNTIGSSLVISPGETLMTLRLYKYSTSNSSHGYEGVQENFTWVFVAANRAALQAGN

SEQ ID NO: 33 - protein, 16 aa, Artificial Sequence
  Portion of wild type pilin domain
YCAKSQNLGYYLSGTT

SEQ ID NO: 34 - protein, 16 aa, Artificial Sequence
  Portion of homologous adhesin
RCDQTQSVSYTLSGSV

(19) United States
(12) Patent Application Publication
(10) Pub. No.: US 2024/0165214 A1
(43) Pub. Date: May 23, 2024

(54) COMPOSITIONS AND METHODS FOR STABILIZATION OF ALLOSTERIC PROTEINS

(71) Applicant: UNIVERSITY OF WASHINGTON, SEATTLE, WA (US)

(72) Inventors: Evgeni V. Sokurenko, SEATTLE, WA (US); Dagmara Kisiela, SEATTLE, WA (US); Pearl Magala, SEATTLE, WA (US); Wendy Evelyn Thomas, SEATTLE, WA (US); Rachel E. Klevit, SEATTLE, WA (US)

(73) Assignee: UNIVERSITY OF WASHINGTON, SEATTLE, WA (US)

(21) Appl. No.: 18/547,644

(22) PCT Filed: Feb. 25, 2022
(86) PCT No.: PCT/US22/70837

§ 371 Date: Aug. 23, 2023

Related U.S. Application Data
(60) Provisional application No. 63/200,285, filed on Feb. 26, 2021.

(51) Int. Cl. A61K 39/108; C07K 1/00; C07K 14/245; C12N 1/20

(52) U.S. Cl. CPC A61K 39/0258; C07K 1/006; C07K 14/245; C12N 1/205; C12R 2001/19

(57) ABSTRACT

Methods that stabilize allosteric cell attachment proteins in either an active or inactive conformation enable toggling a protein between conformational states by restricting the repacking of side chains that occurs during the conformational transition. This restriction traps the protein in the desired high affinity or low affinity state. An allosteric cell attachment protein stabilized in an ‘active’ (high-affinity) or ‘inactive’ (low-affinity) conformation is stabilized by the substitution of a hydrophobic amino acid residue with a charged amino acid, in some embodiments, the attachment protein is bacterial. In some embodiments, the attachment protein is viral, in some embodiments, the protein is a bacterial adhesin.

This application claims benefit of U.S. provisional patent application No. 63/200,285, filed Feb. 26, 2021, the entire contents of which are incorporated by reference into this application.

### ACKNOWLEDGEMENT OF GOVERNMENT SUPPORT

This invention was made with government support under Grant No. R01 AI119675, awarded by the National Institutes of Health. The government has certain rights in the invention.

### REFERENCE TO A SEQUENCE LISTING SUBMITTED VIA EFS-WEB

The content of the ASCII text file of the sequence listing named “UW75WOU1_seq” which is 49 kb in size was created on Feb. 24, 2022, and electronically submitted via EFS-Web herewith the application is incorporated herein by reference in its entirety.

### BACKGROUND OF THE INVENTION

Receptor-ligand interactions are essential for cell adhesion, molecular transport, signal transduction, enzymatic catalysis, etc. Most ligand-binding proteins adopt both ‘inactive’ and ‘active’ conformations and the transition between the two states is usually allosterically controlled. Inactive states are characterized by low-affinity ligand binding, while active states have much higher affinity towards the ligand. The conformational kinetics of the allosteric states and the dynamic mechanisms of ligand recognition have been difficult to decipher due to a lack of tools that can block the transitions without favoring one state over the other.

FimH protein is the most prevalent adhesin of Escherichia coli and a prototypic allosteric receptor protein. It is positioned on the tip of hair-like fimbriae (or pili) and binds to mannosylated cell surface glycoproteins. FimH has two domains—an N-terminal lectin domain (LD) that contains the mannose-binding pocket and a C-terminal pilin domain (PD) that anchors the adhesin to the fimbriae. It is the LD that assumes two conformational states. In the inactive, low-affinity state (LAS), the mannose-binding pocket is wide open due to a structural compression of the β-sandwich domain. The LAS is stabilized by interactions between the LD and the PD, on the opposite side from the binding pocket. In contrast, the binding pocket of the active, high-affinity state (HAS) is more closed and the domain adopts a more elongated β-sandwich conformation, in which LD/PD contacts are disrupted. Ligand binding to FimH induces the transition from LAS to HAS, i.e. adhesin activation, through conformational changes across the LD that favor its separation from the PD. Similarly, removal of the PD to create an isolated LD or structural alteration that leads to disruption of the interdomain interface favors the HAS. The allosteric properties of FimH enable it to form catch bonds-interactions whose lifetimes increase under shear-induced tensile force that facilitates separation of LD and PD. However, while endpoint structural differences between the inactive and active states of LD are known, the key molecular events underlying allosteric transition kinetics remain unclear.

There remains a need for tools that can block the transitions of allosteric proteins without favoring one state over the other. Locked conformations of bacterial adhesins, for example, can be used to inhibit adhesin function, and to develop more effective antibodies, for example, which can be used to disrupt biofilm formation and treat pathogenic infections, as well as prevent infection via contaminated biomedical devices.

### SUMMARY OF THE INVENTION

The invention meets these needs and others by providing methods and compositions that stabilize allosteric cell attachment proteins in either an active or inactive conformation. This stabilization strategy effectively enables one to toggle a protein between conformational states by restricting the repacking of side chains that occurs during the conformational transition. This restriction traps the protein in the desired high affinity or low affinity state. The repacking of side chains depends on a “toggle residue” that changes position in different allosteric forms.

In one embodiment, provided is an allosteric cell attachment protein stabilized in an ‘active’ (high-affinity) or ‘inactive’ (low-affinity) conformation, whereby the conformation is stabilized by the substitution of a hydrophobic amino acid residue with a charged amino acid. In some embodiments, the attachment protein is bacterial. In some embodiments, the attachment protein is viral. In some embodiments, the protein is a bacterial adhesin.

In some embodiments, the substitution stabilizes the protein in an active conformation. In some embodiments, the substitution stabilizes the protein in an inactive conformation. In some embodiments, the hydrophobic amino acid residue is alanine, isoleucine, leucine, methionine, phenylalanine, tyrosine, or valine. In some embodiments, the substitution comprises a charged amino acid substituted for an aliphatic or aromatic amino acid. In some embodiments, the substitution comprises a charged glutamic acid or lysine substituted for a leucine, tyrosine, phenylalanine, and/or a valine. In some embodiments, the protein is Escherichia coli (E. coli) fimbrial adhesin FimH (SEQ ID NO: 1, 2, or 4), and the substitution is at L34, V35, or Y64. In some embodiments, the protein is E. coli fimbrial adhesin FmIH (SEQ ID NO: 3), and the substitution is at L32, V33, and F63. Polynucleotides encoding one or more proteins described herein are also provided. The polynucleotide can be in the form of a vector capable of expressing the protein.

Also provided is an immunogenic composition comprising the protein (or encoding polynucleotide) described herein, and, optionally, an adjuvant. Such compositions can be used, for example, to elicit an immune response directed against the cell attachment protein. In some embodiments, provided is a method of producing antibodies directed against a cell adhesion protein or cell attachment protein. The method comprises administering the immunogenic composition to a subject; obtaining a sample of blood from the subject; and isolating antibodies from the sample of blood. In some embodiments, the immunogenic composition comprises a protein stabilized in an inactive conformation. In some embodiments, the antibodies are neutralizing antibodies.

Further provided is a method of stabilizing an allosteric cell adhesion protein in an active or inactive conformation. In some embodiments, the method comprises substituting a hydrophobic amino acid residue with a charged amino acid. In some embodiments, the protein is a bacterial adhesin. In some embodiments, the hydrophobic amino acid residue is alanine, isoleucine, leucine, methionine, phenylalanine, tyrosine, or valine. In some embodiments, the substitution stabilizes the protein in an inactive conformation. In some embodiments, the substitution stabilizes the protein in an active conformation. In some embodiments, the substitution comprises a charged amino acid substituted for an aliphatic or aromatic amino acid. In some embodiments, the protein is Escherichia coli (E. coli) fimbrial adhesin FimH (SEQ ID NO: 1, 2, or 4), and the substitution comprises L34, V35, or Y64. In some embodiments, the protein is E. coli fimbrial adhesin FmIH (SEQ ID NO: 3), and the substitution comprises L32, V33, and F63. In some embodiments, the charged amino acid is a charged glutamic acid or a charged lysine.

The protein can comprise the full length protein or a portion thereof that includes the relevant domain, such as a lectin binding domain. In some embodiments, the protein or portion thereof has an amino acid sequence of a structural homolog of FimH lectin domain shown in FIG. 14 (any one of SEQ ID NOs: 4-32). In some of these embodiments comprising a homolog shown in FIG. 14, the substitution is at one or more of the residues indicated as homologous to L34, V35 and/or Y64 positions in FimH^{wt}, as indicated in FIG. 14.

### BRIEF DESCRIPTION OF THE DRAWINGS

FIG. 1 is a schematic illustration of the core repacking model of the allosteric transition induced by domain separation.

FIGS. 2A-2G. Recognition of FimH variants by mAb21 and mAb824. Cartoon representation of LAS, intermediate and HAS conformations (2A) and FimH^{FocH} mutant variant (2B) of FimH adhesin. The putative locations of mAb21 and mAb824 epitopes are indicated as cross-hatch and striped shapes, respectively; ±SEM. (2C) Binding of mAb21 in the absence or presence of 1% αmm to plate-coated fimbriae carrying different variants of FimH adhesin; ±SEM. (2D) HRP binding to plate-coated fimbriae in the absence of mAb21 and upon forming a complex with mAb21 in the presence of mannose; ±SEM. (2E) Binding of mAb824 in the absence or presence of 1% αmm to plate-coated fimbriae carrying different variants of FimH adhesin; ±SEM. (2F-2G) Binding of mAb21 to plate-coated FimH^{wt} fimbriae in the presence of different concentration of αmm; and upon no treatment (solid line with open circle) or pre-treatment with αmm (solid line with triangle), Fab824 (solid line with open square), and Fab824 and αmm together (dashed line with open triangle), as designated in the figure. Data (2C-2G) are mean absorbance values at 650 nm ±SEM, n=3.

FIGS. 3A-3E. Epitope of mAb824 mapped on the FimH lectin domain structure. (3A) Views of crystal structures of the FimH LD in the LAS (PDB code 4xo9) and HAS (PDB code 4xo8) conformations. Residues whose mutation affected mAb824 binding are labeled and shown as spheres. Ligand-binding pocket residues on the “top” of the lectin domain are shown as dark grey spheres and the interdomain region residues on the “bottom” are shown as light grey spheres. (3B) Surface rendition of LD (PDB code 3zpd) showing residues with large perturbations in the NMR spectrum upon Fab824 binding (lighter shading). Methyl residues that do not exhibit large perturbations are shown in darker shading. The mAb824 epitope determined by mutagenesis is shown in the middle area. (3C) The putative structural epitope of mAb824 mapped on an LD HAS structure (PDB code 3zpd). Residues immediately bordering the functional epitope and the NMR-determined patch are marked with lighter shading (N23, N33, D37, T74, K76, S78, P83, T86, E89, R92, V93, V105, Y108). (3D) The V35/L34 and Y64 solvent-core switches. Ribbon representation of FimH lectin domain in LAS (PDB code 4xo9) and HAS (PDB code 4xo8) with the alternate orientations of V35 (center of 3D and right in 3E) and L34 (darkest stick). Y64 is left, mid-toned sticks.

FIGS. 4A-4B. ¹³C-methyl TROSY HMQC spectra showing perturbations in the lectin domain due to Fab824 binding. The spectrum of free LD is shown in black and the lectin domain in complex with Fab824 is shown in gray. The peaks are not fully filled in to provide clarity. Examples of the largest perturbations are highlighted in boxes. Note that each methyl group of a sidechain gives its own peak (i.e., two peaks/residue for Ile, Leu, Val). (4B) Expanded regions of the NMR spectra showing examples of residues with large perturbations.

FIGS. 5A-5B. Conformational changes during a simulation of LAS to HAS transition. (5A) SASA of L34 and V35 side chains. The values are averaged over a 0.5 ns time window. Vertical dashed lines indicate the time point when L34 flips in (long-short dashed line) and the time point when V35 flips out (short dashed line), respectively. Flipping in or out, respectively, was defined when the SASA of the respective side chain (in the 0.5-ns time averaged values) became less than or larger than 2 Å², respectively. (5B) Distances between atoms involved in the backbone hydrogen bonds between V36 and L107. Horizontal dashed line [symbol] indicates the distance cutoff used to define a hydrogen bond. i.e., 2.7 Å.

FIGS. 6A-6D. Effect of V35A and L34A mutations. Experiments measure HAS-specific antibody, mAb21, binding FimH^{wt} and the two alanine mutants as a function of αmm. (6A) mAb21 binding to the three FimH variants. (6B) Effect of Fab824 pre-treatment of FimH^{L34A} on mAb21 binding. (6C) Effect of Fab824 pre-treatment to FimH^{V35A} on mAb21 binding. (6D) Recognition of FimH^{wt} and the mutants by mAb21 following pre-incubation of FimH with Fab824 in the presence of 1% αmm and extensive washing from αmm before mAb21 addition. Data (6A-6D) are mean absorbance values at 650 nm ±SEM, n=at least 2 independent experiments.

FIGS. 7A-7C. Effect of mutation of the switch residues on FimH conformational change and function. (7A and 7C) Presence of the HAS conformation of FimH^{wt} and mutants was detected by the HAS-specific mAb21 antibody in ELISA assays. Plate-immobilized fimbriae carrying wild type and mutated variants of FimH were probed with mAb21 in the absence and presence of serially diluted αmm. The data are absorbance values at 650 nm. (7B) Relative binding of the mannose-rich glycoprotein (horseradish peroxide, HRP) to the plate-immobilized fimbriae in the absence and presence of 1% αmm. Data (A-C) are mean±SEM, n=at least 2 independent experiments.

FIGS. 8A-8B. Residues undergo changes in solvent-accessible surface area (SASA) between LAS and HAS of FimH. (8A) The difference in solvent-exposed surface area (ΔSASA) calculated individually for each residue using LAS and HAS conformers of FimH (PDB code 4xo9 and 4xo8, respectively). FimH residues whose SASA changed by 2-fold and at least 20 Å² are shown. The regions that exhibit substantial conformational differences in atomic-level structures of FimH LD LAS and HAS are indicated with differential shading. Mannose-contacting residues are marked by asterisks. (8B) Cα atoms of FimH LD residues highlighted in 8A are shown as spheres on LAS and HAS structures. Residues that transition from exposed-to-buried (downward-facing bars in 8A) are mapped on the LAS conformer (PDB code 4xo9) and those that go from buried to exposed (upward-facing bars in 8A) are mapped on the HAS structure (PDB code 4xo8).

FIG. 9. Toggle switches are conserved in the FimH homologue, FmIH adhesin. Sequence alignment of FimH (SEQ ID NO: 4) and FmIH (SEQ ID NO: 3) lectin domains can be found in the text below (see Detailed Description—Sequences), in which identical residues are indicated with dots. Adhesion mediated by mutant variants of FmIH adhesin in the absence and presence of 1% galactose to surface-coated asialofetuin. Data are mean absorbance values at 600 nm ±SD, n=1 representative experiment.

FIGS. 10A-10C. Analysis of mAb824 binding to fimbrial form of FimH^{wt} and FimH^{FocH}. (10A) Dose-dependent mAb824 binding in the absence and the presence of 1% αmm to surface-immobilized fimbriae bearing different variant of FimH by ELISA. Data are mean±SEM, n=3. (10B) Binding of mAb824 (biotinylated) to PBS-treated- and mAb21-complexed FimH^{wt} fimbriae. (10C) Binding of FimH fimbriae (at 25-100 nM concentrations) to probe-immobilized mAb824 in the absence [symbol] and presence [symbol] of 1% αmm measured by biolayer interferometry (BLI). Kinetic data were globally fitted (black lines) to a 1:1 Langmuir binding model (Prism 6, GraphPad) and obtained kinetic parameters are reported in the text. Data from one representative experiment are shown.

FIGS. 11A-11B. Effect of mAb824 on FimH binding kinetics. Kinetics of FimH^{wt} (11A) and FimH^{FocH} (11B) fimbriae binding to ribonuclease B (RNaseB) measured by biolayer interferometry. Recorded sensorgrams show binding of isolated fimbriae to RNaseB (biotinylated) captured on a streptavidin-coated probe. (11A) Binding of fimbriae (at 50 nM and 100 nM concentrations) in the absence [symbol] , and the presence of Fab824 [symbol] or αmm [symbol] or amm [symbol] . (11B) Binding of fimbriae (at 50 nM and 100 nM concentrations) in the absence [symbol] , and the presence of Fab824 [symbol] . The binding data were globally fitted (black lines) to two-state conformation change (FimH^{wt}) (Yakovenko O, et al. 2015 Proc Natl Acad Sci USA 112: 9884-9889) (11A) and a 1:1 Langmuir (FimH^{FocH}) (11B) binding models.

FIG. 12. Red blood cell (RBC) aggregation by FimH-expressing bacteria under rocking conditions; the 10 minutes time point. (Left panel) RBC aggregation without pre-incubation with mAb824, with serial dilutions of bacteria (1:1 corresponds to the OD=1.0); (Right panel) RBC aggregation upon pre-incubation with mAb824; at the highest bacterial dose.

FIGS. 13A-13B. Correlation between SASA side chains of the switching residues along the 5.8-μs long MD simulation. (13A) A previously published MD simulation (Interlandi G, Thomas W E 2016 Proteins 84: 990-1008) performed at 330 K on the Anton supercomputer, a resource dedicated to the production of long MD trajectories, was re-analyzed to study correlations between time series of SASA values of side chains that are observed to change between surface and core orientations when comparing X-ray structures of LAS and HAS. A total of 25 side chains were compared pairwise and the Pearson's linear correlation coefficient was calculated between the 240-ns time averages of their SASA values using the program xmgrace. Color scale indicates the strength of the correlation. (13B) Time course of the solvent accessibility of V35 and Y64 along the 5.8-μs long MD simulation. Plotted are running averages over a time window of 240 ns.

FIG. 14. Sequence alignment of 29 representative structural homologues of E. coli FimH lectin domain retrieved from the Pfam database. (SEQ ID NOs: 4-32) The identity/similarity of sequences is marked by red/grey shading. Sequence numbering (#1-#29, corresponding to SEQ ID NOs: 4-32, respectively) is the same as in Table 5. L34, V35 and Y64 positions in FimH^{wt} (sequence #1) are marked by asterisks.

FIG. 15. Free energy diagram for FimH conformational changes in the absence and presence of antibodies. In the absence of mannose, LAS predominates while HAS predominates in the presence of mannose. mAb21 binding further stabilizes HAS. mAb824 traps both HAS and LAS by increasing the transition-state free energy (ΔG^{t}) required to transit between the two states, lowering the frequency of the switch in both directions.

FIGS. 16A-16G. Phenotypic profiles of the FimH variants. (16A) Schematic diagrams of the three conformations of FimH resolved to date by X-ray crystallography; (16B) Bacterial adhesion to yeast mannan-coated surface; the binding is >95% inhibited by 1% mannose; (16C) Binding of soluble HRP to the surface-immobilized fimbriae; the binding is >95% inhibited by 1% mannose; (16D) Binding of HAS-specific mAb21 to the fimbriae in absence of mannose; (16E) Same as (16D) but in the presence of 0.1% mannose; (16F) Binding of mAb824 to the fimbriae; the binding is not affected by 1% mannose; (16G) Binding of soluble HRP to the fimbriae pre-treated with mAb824; the binding is >95% inhibited by 1% mannose; (16B-16E) Y axis is the OD absorbance level at 650 nm wavelength.

FIGS. 17A-17C. Crystal structure of the fimbrial tip expressing LD^{wt}-PD^{FocH} variant of FimH (PDB code 7SZO). (17A) Full tip structure; (17B) Overlap of the LD^{wt}-PD^{FocH} structure (7SZO chain H) with the tip structure of LD^{wt}-PD^{wt} variant of FimH (from the PDB code 3JWN chain H); (17C) Amino acid residues replaced in LD^{wt}-PD^{FocH} relatively to LD^{wt}-PD^{wt}. Dark spheres are residues located within the inter-domain interface. Lighter spheres are the residues outside the inter-domain interface.

FIGS. 18A-18B. Inter-domain side chain contacts and hydrogen bonds. (18A) LD^{wt}-PD^{wt} (PDB code 3JWN chain H); (18B) LD^{wt}-PD^{FocH} (PDB code 7SZO chain H). A side chain contact is defined using a cutoff of 6 Å on the distance between the centers of geometry of two side chains. A hydrogen bond is defined when the H . . . O distance is less than 2.7 Å and the D-H . . . O angle is at least 120°. Dotted line are the contacts between A188 in PD^{wt} or D188 in PD^{FocH} with V28 and V30 in LD^{wt}.

FIGS. 19A-19D. Time course of mAb21 binding to the surface-immobilized fimbriae in the absence or presence of soluble mannose. Different FimH variants are shown. Y axis is the OD absorbance level at 650 nm wavelength.

FIGS. 20A-20D. Modeling the mAb21 binding time course data. (20A) Schematic diagram of the model used to estimate the conformational constants. (20B) The estimated equilibrium constants (K_{eq}); (20C) The estimated activation constants (k_{act}), and (20D) deactivation constants (k_{deact}). The cross-hatched circles indicate the mean of estimates calculated for independent experiments, and the cross-hatched bars associated with cross-hatched circles indicate the confidence interval as determined by plus or minus one standard error of the mean. In conditions for which multiple values of the rate constants fit the data equally well, the cross-hatched bars indicate the range of values that provided the best fit, while the open circles indicate the estimate of the constants assuming that k_{act} is the same (average) as for the variants for which the kinetics could be measured. Note that no values are reported for LD^{wt}-PD^{wt} in the absence of mannose due to the lack of measurable mAb21 binding.

FIG. 21. Time course of soluble HRP binding to the surface-immobilized fimbriae. Y axis is the OD absorbance level at 650 nm wavelength. For the conditions in the presence of mAb824, the antibody was incubated with FimH prior to the addition of HRP.

FIGS. 22A-22B. Red blood cells agglutination assays. (22A) Under static conditions in U-bottom microtiter plates. Top—positive result (‘rosette’ formation); bottom—negative result. (20B) Under dynamic conditions on a slide. Top—positive result; bottom—negative result.

FIG. 23. Free energy landscapes of the allosteric conformational change. In this illustration, the energy is plotted relative to the energy of the LAS state for each curve. The heights of the various states in the curves reflect both previously established knowledge and the rate and equilibrium constants for LAS/HAS transition estimated.

FIG. 24. Persistence of side chain contacts involving side chains that differ between PD^{wt} and PD^{FocH}. Shown are time series of side chain contacts formation in two simulations with LD^{wt}-PD^{wt} and two runs with LD^{wt}-PD^{FocH}. The ratio of frames where a side chain is formed in a simulation is reported on the right side. Higher values (0.71-0.99) denote side chain contacts that are formed in at least 66% of the simulation frames, in which case a contact is said to be persistent in that particular simulation.

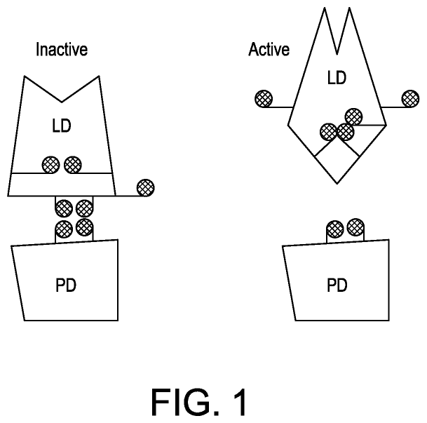

### DETAILED DESCRIPTION

The methods and compositions that stabilize allosteric cell attachment proteins in either an active or inactive conformation, as described herein, enable one to toggle a protein between conformational states. This toggling is achieved by restricting the repacking of side chains that occurs during the conformational transition, thereby trapping the protein in the desired high affinity or low affinity state. The method is based on a discovery that the orientation of certain amino acid residues, e.g. “toggle residues”, is critical for the conformational transition between inactive and active states. This provides a simpler, more streamlined means for stabilizing a conformation than, for example, multiple disulfide bridge mutations. Moreover, targeting toggle residues for the conformational stabilization provides a rational approach for allosteric stabilization without requiring knowledge of the specific structural differences between the states or without requiring possession of a crystal structure. Strong effects have been observed with a single substitution, and multiple substitutions can be made. In addition, the stabilized proteins described herein provide more effective immunogens and better targets for inhibitors.

Allosteric cell attachment proteins are suitable for this approach to stabilization of their conformation, as exemplified by the bacterial adhesin FimH, because they exist in multiple functional states, and they contain shifting side chains that participate in the core re-packing process responsible for allosteric change. These shifting side chains can thus be manipulated to make them less prone to shift from or inside the core, for example, by increasing the shifting amino acid hydrophilicity or, alternatively, hydrophobicity. For example, by substituting a shifting hydrophobic amino acid to hydrophilic amino residue or, alternatively, a shifting hydrophilic or less hydrophobic amino acid to a more hydrophobic amino acid.

### Definitions

As used herein, “charged amino acid” refers to any of the following: Arginine (Arg or R); Lysine (Lys or K); Aspartic acid (Asp or D); or Glutamic acid (Glu or E). In some embodiments, the charged amino acid is E or K.

As used herein, “aliphatic amino acid” refers to any of the following: Alanine (Ala or A); Isoleucine (Ile or I); Leucine (Leu or L); Valine (Val or V); and Glycine (Gly or G).

As used herein, “aromatic amino acid” refers to any of the following: tryptophan (Trp or W); tyrosine (Tyr or Y); and Phenylalanine (Phe or F).

Examples of amphipathic amino acids include: tryptophan (Trp or W); tyrosine (Tyr or Y); and methionine (Met or M).

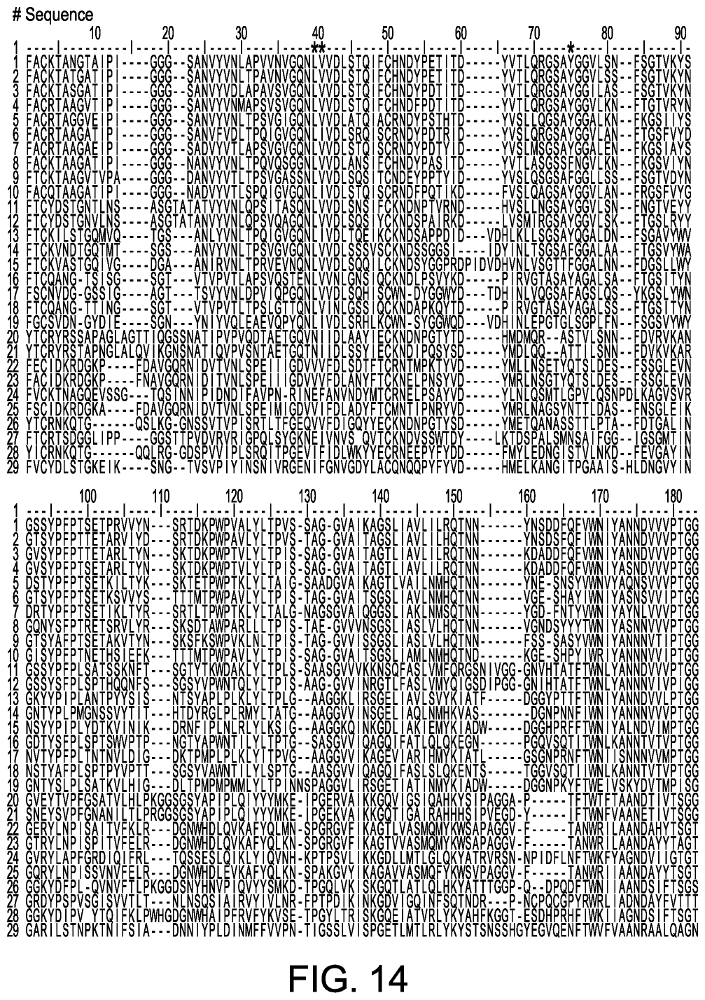

As used herein, “hydrophobic amino acids” refers to amino acids having little to no polarity, including any of: Alanine (Ala or A); Isoleucine (Ile or I); Leucine (Leu or L); methionine (Met or M); Phenylalanine (Phe or F); Valine (Val or V); Proline (Pro or P); tyrosine (Tyr or Y); and Glycine (Gly or G). These hydrophobic amino acids serve as “toggle residues” in that they change position when the protein takes on different allosteric forms. Some toggle residues move in or out of the protein core versus the protein surface. Some examples of toggles in FimH homologues are listed in FIG. 14.

As used herein, “antibody” refers to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv); and multispecific antibodies formed from antibody fragments.

As used herein, “specifically binds” refers to the binding of an antibody to a specific protein or target which is present amongst a heterogeneous population of proteins. Hence, when present in specific immunoassay conditions, the antibodies bind to a particular protein target, such as FimH, and do not bind in a significant amount to other proteins present in the sample.

As used herein, “identical” means, with respect to amino acid sequences, that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This type of substitution can be referred to as a conservative substitution. Preferably, a conservative substitution of any of the amino acid residues contained in a given amino acid sequence, these changes have no effect on the binding specificity or functional activity of the resulting antibody when compared to the unmodified antibody.

As used herein, “corresponding position” refers to an amino acid residue that is present in a second sequence at a position corresponding to a specified amino acid residue in a first sequence which is the same position as the position in the first sequence when the two sequences are aligned to allow for maximum sequence identity between the two sequences.

As used herein, “consists essentially of” or “consisting essentially of” means that a polypeptide may have additional features or elements beyond those described, provided that such additional features or elements do not materially affect the ability of the antibody or antibody fragment to have the recited binding specificity. The antibody or antibody fragments comprising the polypeptides may have additional features or elements that do not interfere with the ability of the antibody or antibody fragments to bind to its target and exhibit its functional activity, e.g., disrupting or preventing bacterial adhesion to a mannose-coated surface. Such modifications may be introduced into the amino acid sequence in order to reduce the immunogenicity of the antibody. For example, a polypeptide consisting essentially of a specified sequence may contain one, two, three, four, five or more additional, deleted or substituted amino acids, at either end or at both ends of the sequence provided that these amino acids do not interfere with, inhibit, block or interrupt the role of the antibody or fragment in binding to its target and exhibiting its biological activity.

As used herein, a “heterologous” sequence or a “heterologous” molecule refers to a moiety not naturally occurring in conjunction with a recited sequence or molecule. Representative examples of the heterologous molecule include, but are not limited to, a polypeptide, antibody, epitope, polynucleotide, small molecule or drug. Such heterologous moieties can be useful for improving solubility, delivery, immunogenicity, efficacy, detection, or identification of the recited sequence or molecule. In some embodiments, the heterologous sequence is inert or an unrelated sequence. A moiety which contributes to antibodies of the invention may be chemically modified with one or more functional groups, provided that such functional groups do not interfere with the ability of the antibody or antibody fragment to bind to FimH and disrupt or prevent bacterial adhesion to a mannose-coated surface.

As used herein, “enterobacteria” (or enterobacteriaceae) refers to gram-negative bacteria that express the bacterial Type 1 fimbrial adhesin FimH. Examples of enterobacteria include, but are not limited to, E. coli, K. pneumoniae, K. oxytoca, Shigella, Serratia spp, Enterobacter spp, Citrobacter, Salmonella, Morganella, and Edwardsiella.

As used herein, “inflammatory bowel disease” (or IBD) refers to inflammatory conditions of the colon and small intestine, including, but not limited to, Crohn's disease and ulcerative colitis.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, (Remington and Gennaro 1990)).

As used herein, to “prevent” or “treat” a condition means to decrease or inhibit symptoms indicative of the condition or to delay the onset or reduce the severity of the condition.

As used herein, “adjuvant” includes those adjuvants commonly used in the art to facilitate an immune response. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

As used herein, the terms “comprise” or “include”, or variations such as “comprises” or “comprising”, “includes” or “including” mean the inclusion of a recited item or group of items, but not the exclusion of any other item or group of items.

### Sequences

The amino acid sequence of FimH of K12 (SEQ ID NO: 1, upper rows) and UTI89 (SEQ ID NO: 2, lower rows) is shown below.

Amino Acid Sequence of FimH of K12 (upper rows) and UTI89 (lower rows):

```
                  10 20 30 40 50 60 70 80
....|....|....|....|....|....|....|....|....|....|....|....|....|....|....|....|
FACKTANGTAIPIGGGSANVYVNLAPVVNVGQNLVVDLSTQIFCHNDYPETITDYVTLQRGSAYGGVLSNFSGTVKYSGS
..........................A..................................A......S........N..
                  90 100 110 120 130 140 150 160
....|....|....|....|....|....|....|....|....|....|....|....|....|....|....|....|
SYPFPTTSETPRVVYNSRTDKPWPVALYLTPVSSAGGVAIKAGSLIAVLILRQTNNYNSDDFQFVWNIYANNDVVVPTGG
................................................................................
                170 180 180 200 210 220 230 240
....|....|....|....|....|....|....|....|....|....|....|....|....|....|....|....|
CDVSARDVTVTLPDYPGSVPIPLTVYCAKSQNLGYYLSGTTADAGNSIFTNTASFSPAQGVGVQLTRMGTIIPANNTVSL
................................................................................
                250 260 270 280
....|....|....|....|....|....|....|....|
GAVGTSAVSLGLTANYARTGGQVTAGNVQSIIGVTEVYQ* SEQ ID NO: 2
.......................................* SEQ ID NO: 1
```

A sequence alignment of FimH lectin domain (aa 1-160; SEQ ID NO: 4) and FmIH (aa 1-160; SEQ ID NO: 3) is shown below.

```
                            10 20 30 40 50 60 70
          ....|....|....|....|....|....|....|....|....|....|....|....|....|....|
FimH FACKTANGTAIPIGGGSANVYVNLAPVVNVGONEVVDLSTQIFCHNDYP-ETITDYVTLQRGSAYGGVLS
FmlH .S.NDVG.SS--..A.TTS.....D..IQP.........QH.S.W...GGWYD..HIN.VQ...FA.S.Q
                            80 90 100 110 120 130 140
          ....|....|....|....|....|....|....|....|....|....|....|....|....|....|
FimH NFSGTVKYSGSSYPFPTTSETPRVVYNSRTDKPWPVALYLTPVSSAGGVAIKAGSLIAVLILRQTNNYNS
FmlH SYK.SLYWNNVT....L.TN.NVLDIGDK.PM.L.LK..I...GA....V....EV..RIHMYKIATLG.
                          150 160
          ....|....|....|....|..
FimH DD-FQFVWNIYANNDVVVPTGG SEQ ID NO: 4
FmlH GNPRN.T...IS..S..M.... SEQ ID NO: 3
```

Additional sequences of structural homologs of E. coli FimH lectin domain are listed in FIG. 14 (SEQ ID NOs: 5-32).

### Conformationally Stabilized Allosteric Proteins

An allosteric cell attachment protein can be stabilized in an ‘active’ (high-affinity) or ‘inactive’ (low-affinity) conformation. Stabilization is achieved by reducing or, alternatively, increasing, hydrophobicity of switching residues via the amino acid substitution. In some embodiments, the attachment protein is bacterial. In some embodiments, the attachment protein is viral. In some embodiments, the protein is a bacterial adhesin. Common to the allosteric proteins that can be stabilized in high-affinity or low-affinity conformation as described herein is the presence of shifting side chains that participate in re-packing the protein core by flipping between discrete positions. By altering the ability of the side chains to participate in the core re-packing process, the substitution of a hydrophobic amino acid (“toggle”) residue with a charged amino acid or, alternatively, less hydrophobic amino acid to more hydrophobic amino acid results in a protein that remains stabilized in one conformation or another, depending on the preferred orientation of the shifting residue in one conformation or another.

In some embodiments, the substitution stabilizes the protein in an active conformation. In some embodiments, the substitution stabilizes the protein in an inactive conformation. In some embodiments, the hydrophobic amino acid residue is alanine, isoleucine, leucine, methionine, phenylalanine, tyrosine, or valine. In some embodiments, the substitution comprises a charged amino acid substituted for an aliphatic or aromatic amino acid. In some embodiments, the substitution comprises a charged glutamic acid or lysine substituted for a leucine, tyrosine, phenylalanine, and/or a valine. In some embodiments, the protein is Escherichia coli (E. coli) fimbrial adhesin FimH (SEQ ID NO: 1, 2, or 4), and the substitution is at L34, V35, or Y64. In some embodiments, the protein is E. coli fimbrial adhesin FmIH (SEQ ID NO: 3), and the substitution is at L32, V33, and F63. Thus, specific variants of SEQ ID NO: 1, 2 or 4, such as L34E, L34K, V35E, V35K, Y64E, Y64K, and combinations thereof are contemplated. Likewise, variants of SEQ ID NO: 3, such as L32E, L32K, V33E, V33K, F63E, F63K, and combinations thereof, are also examples of contemplated embodiments. The protein can comprise the full length protein or a portion thereof that includes the relevant domain, such as a lectin binding domain. In some embodiments, the protein or portion thereof has an amino acid sequence of a structural homolog of FimH lectin domain shown in FIG. 14 (any one of SEQ ID NOs: 4-32). In some of these embodiments comprising a homolog shown in FIG. 14, the substitution is at one or more of the residues indicated as homologous to L34, V35 and/or Y64 positions in FimH^{wt}, as indicated in FIG. 14.

Polynucleotides encoding one or more proteins or portion thereof described herein are also provided. The polynucleotide can be in the form of a vector capable of expressing the protein or relevant portion thereof (e.g., lectin domain). The polynucleotide can be formulated for delivery into a cell, tissue, or organism, permitting expression of the encoded protein by a host cell or organism.

Also provided is an immunogenic composition comprising the protein (or encoding polynucleotide) described herein, and, optionally, an adjuvant. Such compositions can be used, for example, to elicit an immune response directed against the cell attachment protein. In some embodiments, the composition is used to neutralize or inactivate the ability of a cell attachment protein to mediate cell attachment under one condition or another, such as, for example, pH, temperature, or mechanical tensile force.

### Methods

In some embodiments, provided is a method of producing antibodies directed against a cell adhesion protein or cell attachment protein. The method comprises administering the immunogenic composition to a subject; obtaining a sample of blood from the subject; and isolating antibodies from the sample of blood. In some embodiments, the immunogenic composition comprises a protein stabilized in an inactive conformation. In some embodiments, the antibodies are neutralizing antibodies. Also provided are methods of eliciting an immune response to a cell attachment protein in a subject, and methods of neutralizing cell attachment proteins in a subject, the method comprising administering an immune composition as described herein to the subject.

Antibodies to bacterial adhesin produced in this manner can be used to prevent attachment of bacteria to a mannose-coated surface, and also detach bacteria already attached to a mannose-coated surface. Such antibodies can be used to disrupt or prevent the attachment of a single layer of bacteria to a mannose-coated surface, or to disrupt or prevent the formation of a multilayer biofilm. Such antibody and immune compositions of the invention can thus be used in methods to inhibit, prevent, or reverse the colonization of a surface with enterobacteria that express the bacterial Type 1 fimbrial adhesin FimH, to inhibit or prevent infection of a cell by enterobacteria that express the bacterial Type 1 fimbrial adhesin FimH, such as, for example, uropathogenic E. coli, to treat a bacterial infection in subject in need thereof, and to treat or prevent inflammatory bowel disease (IBD).

Further provided is a method of stabilizing an allosteric cell adhesion protein in an active or inactive conformation. In some embodiments, the method comprises substituting a hydrophobic amino acid residue with a charged amino acid. In some embodiments, the protein is a bacterial adhesin. In some embodiments, the hydrophobic amino acid residue is alanine, isoleucine, leucine, methionine, phenylalanine, tyrosine, or valine. In some embodiments, the substitution stabilizes the protein in an inactive conformation. In some embodiments, the substitution stabilizes the protein in an active conformation. In some embodiments, the substitution comprises a charged amino acid substituted for an aliphatic or aromatic amino acid. In some embodiments, the protein is Escherichia coli (E. coli) fimbrial adhesin FimH (SEQ ID NO: 1, 2, or 4), and the substitution comprises L34, V35, or Y64. In some embodiments, the protein is E. coli fimbrial adhesin FmIH (SEQ ID NO: 3), and the substitution comprises L32, V33, and F63. In some embodiments, the charged amino acid is a charged glutamic acid or a charged lysine. In some embodiments, the allosteric cell attachment protein comprises a beta sheet structure.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, murine, porcine, and ovine animals as well as other veterinary subjects. Typical patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The compositions are administered in any suitable manner, often with pharmaceutically acceptable carriers. Suitable methods of administering cells in the context of the present invention to a patient are available, and, although more than one route can be used to administer a particular composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as enterobacterial infection, the physician needs to evaluate the progression of the disease, and any treatment-related toxicity.

### Kits

For use in the methods described herein, kits are also within the scope of the invention. Such kits can comprise a package or container that is compartmentalized to receive one or more containers such as vials, tubes, and the like, each of the container(s) comprising one of the separate elements (e.g., antibodies, proteins; polynucleotides; carriers) to be used in the method. Typically, the kit comprises one or more proteins or polynucleotides of the invention. The kit further comprises one or more containers, with one or more elements stored in the containers. The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for use. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1: Toggle Switch Residues Control Allosteric Transitions in Bacterial Adhesins by Participating in a Concerted Repacking of the Protein Core

Critical molecular events controlling conformational transitions in most allosteric proteins are ill-defined. The mannose-specific FimH protein of Escherichia coli is a prototypic bacterial adhesin that allosterically switches from an ‘inactive’ to ‘active’ conformation upon mannose binding and mediates shear-dependent catch bond adhesion. Here we identify a novel type of antibody that acts as a kinetic trap that prevents the transition between conformations in both directions. Disruption of the allosteric transitions significantly slows down the FimH abilities to associate with mannose and completely blocks the bacterial adhesion under dynamic conditions. FimH residues critical for antibody binding form a compact epitope located away from the mannose-binding pocket and structurally conserved in both states. However, the antibody-FimH contact area identified by NMR is substantially larger and contains residues Leu-34 and Val-35 that move between core and surface orientations in opposing directions during the transition. The antibody is unable to trap the conformations if Leu-34 and Val-35 are replaced with a less bulky alanine.

Replacement of Leu-34 with a charged glutamic acid stabilizes FimH in its inactive conformation, while replacement of Val-35 with glutamic acid traps FimH in the active conformation. This Example demonstrates that these residues act as molecular toggle switches and that the bound antibody imposes a steric block to their rotation in either direction, thereby restricting the concerted repacking of side chains that occurs during the conformational transition. Residues homologous to the FimH toggle switches are highly conserved across a diverse family of fimbrial adhesins. Charged replacement of predicted switch residues uncovers that another E. coli adhesin, galactose-specific FmIH, is allosteric and can shift from an inactive to an active state. This Example shows that allosteric transitions in bacterial adhesins depend on toggle switch residues and that an antibody that blocks the switch effectively disables adhesive protein function.

Receptor-ligand interactions are essential for cell adhesion, molecular transport, signal transduction, enzymatic catalysis, etc. Most ligand-binding proteins adopt both ‘inactive’ and ‘active’ conformations and the transition between the two states is usually allosterically controlled. Inactive states are characterized by low-affinity ligand binding, while active states have much higher affinity towards the ligand (1-3). The conformational kinetics of the allosteric states and the dynamic mechanisms of ligand recognition have been difficult to decipher due to a lack of tools that can block the transitions without favoring one state over the other.

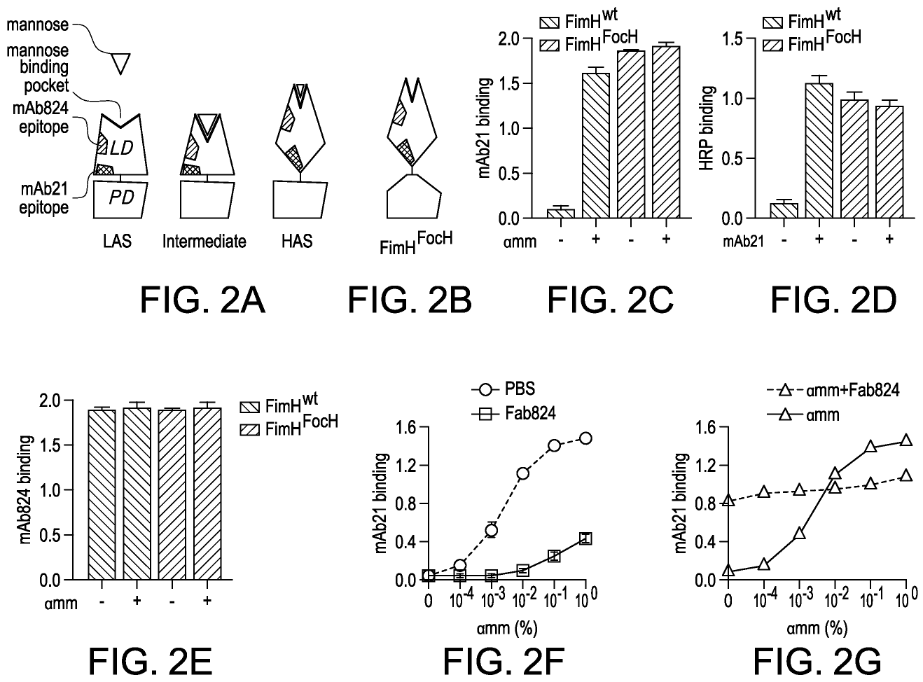

FimH protein is the most prevalent adhesin of Escherichia coli: over 90% of strains express FimH under one or another condition (4). FimH is a prototypic allosteric receptor protein that is positioned on the tip of hair-like fimbriae (or pili) and binds to mannosylated cell surface glycoproteins (5). The protein is composed of two domains: an N-terminal lectin domain (LD) contains the mannose-binding pocket and a C-terminal pilin domain (PD) anchors the adhesin to the fimbriae (6). The LD can assume two end-point conformational states—a low-affinity state (LAS) and a high-affinity state (HAS)—and there is evidence for at least one intermediate state (FIG. 2A). The LAS has a compressed β-sandwich structure and an open mannose-binding pocket. Inter-domain interactions with the PD at the opposite end of the LD from the binding pocket stabilize the LAS (7). In contrast, the binding pocket of the HAS is more dosed, the β-sandwich is more elongated, and LD/PD contacts are disrupted (6). Ligand binding to LAS FimH induces conformational changes to an intermediate state in which the binding pocket is closed but the interdomain interaction is preserved (8) and the end-point HAS in which the LD and PD are separated is achieved via an allosteric transition to (9-11). Importantly, the domain separation and, therefore, transition from the intermediate to HAS is both strongly favored and sustained by mechanical tensile force. Force-induced domain separation may occur on a faster timescale than the mannose-induced separation and is critical during FimH-mediated bacterial adhesion under physiological shear induced by flow conditions. Thus, FimH is able to form a so-called catch bond with its ligand, in which the lifetime of binding increases under shear (9,10,12). The mannose-induced transition may occur on a slower timescale but likely plays a critical role during adhesion under low-shear, static, or equilibrium conditions. Removal of the PD to create an isolated LD or structural alterations that lead to disruption of the interdomain interface in full-length FimH both strongly favor the HAS conformation of LD (7,9). However, while the structural differences between the endpoint LAS and HAS of FimH are well defined, the key molecular events that underlie the allosteric transition remain undefined.

Conformation-specific monoclonal antibodies are powerful tools for studying allosteric receptor proteins. HAS-specific monoclonal antibodies have been identified for FimH, with the representative antibody mAb21 binding to the interdomain region of LD that is exposed in the HAS (10,13). To date, no antibody has been identified that blocks the conformational transition between LAS and HAS in both directions. Here we characterize a novel monoclonal antibody that recognizes both LAS and HAS conformations of FimH. Binding to either state of the adhesin inhibits the allosteric transition in both directions. We propose that antibody binding blocks the ability of particular residues within the LD-antibody interface to switch between solvent-exposed and core-buried orientations, a step we show to be critical for the allosteric transition.

### Materials and Methods

Bacterial Strains, Fimbriae and Reagents

The recombinant Escherichia coli K12 strain (AAEC191A) carrying pPKL114 plasmid containing the entire fim gene cluster from the E. coli strain K12 (but with the inactivated fimH gene) and pGB2-24 plasmid caring fimH^{wt} was described previously (9,10). The fmIH gene was cloned by PCR from E. coli CFT073 strain and inserted into pGB2-24 using ApaL1/Sph1 restriction sites. Isogenic pGB2-24-based plasmids carrying mutated variants of fimH and fmIH were obtained by site-directed PCR mutagenesis using primers comprising specific mutations (13). For type 1 fimbriae expression, bacteria were grown overnight in 1I LB cultures with mild shaking. The type 1 fimbriae were isolated from spun bacterial cultures using a magnesium chloride precipitation method (46). RNase B (Sigma) was biotinylated using Sulfo-NHS-LC-Biotin (Pierce) according to manufacturer's recommendations followed by FPLC purification using Superdex 75 (GE Healthcare Life Sciences).

Antibodies and Fabs

mAb824 and mAb21 were expressed and purified from serum-free hybridoma cell cultures grown in SFM media (Gibco) as described elsewhere (13,47). Briefly, antibodies from culture supernatants were first collected using protein G-agarose (Millipore) followed by size-exclusion chromatography using Superdex 200 (GE Healthcare Life Sciences). Purified mAbs were further digested to Fab fragments using agarose-immobilized ficin (Thermo Fisher Scientific) and separated further by FPLC using Superdex 75 (GE Healthcare Life Sciences).

Epitope Mapping

Binding of mAb824 to a library of purified (isogenic) type 1 fimbriae carrying different mutations in the lectin domain of FimH^{K12} (expressed with wild type-FimH^{K12}- and mutated-FimH^{FocH} pilin domain) was screened by ELISA (13,47). We used an extensive library of FimH mutants compiled in multiple previous studies. In cases when candidate epitope residues were found, more targeted mutagenesis was performed. Fimbriae with FimH^{FocH} adhesin were used as a reference against which binding of the antibody to all lectin domain mutant fimbriae was compared.

NMR Sample Preparation, Data Collection and Visualization

¹⁵N and ¹³C isotopic labeling of the lectin domain was carried out by growing E. coli cells and inducing protein expression (1M IPTG) in minimal media containing ¹³C-glucose and ¹⁵N-ammonium chloride as the carbon and nitrogen sources respectively. The Fab824-lectin domain complex was formed by first immobilizing a 5× excess of lectin domain on a nickel resin via its histidine tag, after which Fab824 was added to the nickel slurry and incubated overnight on a nutator at 4° C. The complex was recovered by washing the nickel resin with 20 mM sodium phosphate buffer pH 8, containing 300 mM sodium chloride (wash buffer) to remove the free Fab824 and then eluting with the wash buffer containing 500 mM imidazole. The elution was concentrated and loaded on a size exclusion column (Superdex 75) to separate the excess lectin domain from the Fab824-lectin domain complex.

NMR Methyl TROSY spectra of the Fab824-lectin domain complex were collected on a Bruker 800 MHz AVANCE spectrometer at 298K. NMR samples were prepared in deuterium oxide with 20 mM sodium phosphate (pH 6), 1 mM EDTA and 100 mM sodium chloride. Data were processed using standard protocols in NMRPipe (48). NMR data were visualized analyzed in NMRviewJ (49), and the FimH lectin domain assignments were obtained from the Biological Magnetic Resonance Databank (BMRB entry 19256).

ELISA Assays

To test antibody binding to FimH, microtiter plate wells were coated with purified fimbriae at a concentration of 0.1 mg/ml in 0.02 M NaHCO₃ buffer for 1 h at 37° C. The wells were washed with PBS and quenched for 20 min with 0.2% (wt/vol) BSA in PBS. The immobilized fimbriae were incubated with serial dilutions of pure mAbs for 1 h, and after washing, bound antibodies were detected with a 1:3,000 diluted HRP-conjugated goat anti-mouse antibody (Bio-Rad). The reaction was developed at room temperature using 3,3′,5,5′-tetramethylbenzidine (TMB, Kirkegaard and Perry Laboratories [KPL]), and absorbance was read at 650 nm. In some experiments, binding of the antibodies was examined in the presence of 1% of αmm. All incubation steps in ELISA assays were performed at 37° C. unless stated otherwise.

To test the effect of αmm or mAb824 on mAb21 binding to fimbrial FimH, immobilized fimbriae were incubated for 45 min with 1% αmm, or 25 μg/ml pure Fab824, or 1% αmm and the Fab together. After washing, 0.2 μg/ml mAb21 was added to wells for 45 min. Bound antibodies were detected with a 1:5,000 diluted HRP-conjugated goat anti-mouse Fc antibody (Sigma-Aldrich). In some experiments, mAb21 at a concentration of 0.5 μg/ml was added to fimbriae-coated wells in the absence or presence of serial dilutions of αmm. For simultaneous mAb824/mAb21 binding test, the fimbrial FimH^{wt} was first complexed with mAb21 antibody in the presence of 1% αmm and after wash with PBS probed with biotinylated mAb824 (used in concentration 0.1 μg/ml). Binding of the latter antibody was detected using 1:5,000 diluted HRP-conjugated streptavidin (Invitrogen).

Red Blood Cell (RBC) Aggregation Assay

Guinea pig RBC (Colorado Serum Company) were washed with PBS three times. 50 μl of RBC suspension (1%) was mixed 1:1 with 50 μl of 4×10⁸ of type 1 fimbriated bacteria in a glass plate in the absence and presence of 1% αmm and continuously mixed at high speed on rotator (Model 260300F, Fisher Scientific) for extended period of time. The aggregation of RBC was visually scored and the time at which noticeable aggregation was observed and reported in minutes (min). In some experiments, bacterial cells were first pre-incubated for 5 min with 67 μg/ml of mAbs in the glass plate and then mixed with RBC suspension in the absence and presence of 1% αmm.

Horseradish Peroxidase (HRP)-Binding Assay

96-well microtiter plates were coated overnight with purified type 1 fimbriae in 0.02 M NaHCO₃ buffer at pH 9.6 (0.1 mg/m). The immobilized fimbriae, after blocking with 0.2% BSA in PBS were incubated with horseradish peroxidase (HRP) at a concentration of 50 μg/ml for 1 h in the absence or presence of 1% αmm. After extensive washing with PBS, bound HRP was detected by adding 100 μl of TMB substrate (Kirkegaard and Perry Laboratories, KPL) and absorbance was read at 650 nm.

Bacterial Binding Assay

FmIH-dependent bacterial adhesion was analyzed as described previously with minor modifications (47). Briefly, Immulon 4HBX microtiter plates (Thermo Electron Corp.) were coated with 20 μg/mL of asialofetuin (Sigma-Aldrich) and plates were quenched with 0.2% BSA in PBS. Bacterial suspensions in PBS (OD540=2) were added to each well in a volume of 100 μl and plates were spun for 2 min at 750×g. After extensive washing, bound bacteria were stained with 0.1% (vol/vol) crystal violet for 20 min at room temperature and were washed several times with water. Then 100 μl of 50% (vol/vol) ethanol was added to each well, and the absorbance at a wavelength of 600 nm was measured. For inhibition, bacterial binding was tested in the presence of 1% galactose (Sigma).

Biolayer Interferometry

The kinetics of antibody binding to fimbrial forms of FimH was determined by using monoclonal antibodies (mAb) captured on anti-mouse IgG biosensors (ForteBio). In brief, hydrated biosensors (FortéBio) were immobilized with purified mAb824 (applied at a concentration of 5 μg/ml in HEPES buffered saline (HBS, pH 7.4)) supplemented with 0.2% BSA until the signal reached ˜0.5 nm. The antibody-immobilized biosensors were washed with 0.2% BSA HBS and allowed to interact with serially diluted purified fimbriae (25-100 nM) in the absence and presence of 52 mM αmm. After the association phase, the dissociation of mAb-fimbriae complexes was performed by moving the biosensors into 0.2% BSA HBS buffer. mAb antibody-captured biosensors in 0.2% BSA HBS buffer (in the absence and presence 52 mM αmm, respectively) were used for single reference subtraction. The collected data were processed using Data Analysis 7.0 software (FortéBio). The data were globally fitted using a 1:1 Langmuir binding model in GraphPad Prism 6 software. Of note, preincubation of fimbriae with αmm (190 g/mol) did not affect sensorgrams recorded for fimbriae binding with the average molecular weight of fimbriae being 15000 kDa.

To test the kinetics of fimbriae binding to RNase B, streptavidin-coated biosensors (SA, FortéBio) were first allowed to bind biotinylated RNase B (applied at a concentration of 10-20 μg/ml in 0.2% BSA HBS). After the baseline step with 0.2% BSA HBS, the RNase B-captured biosensors were exposed to purified fimbriae (50 and 100 nM concentrations) in the absence or presence of 4 μM Fab824 (i.e. in the presence of a 80- and a 40-fold excess of the Fab in relation to purified fimbriae, respectively), or in some experiments 104 mM αmm. The dissociation of the fimbriae-RNase B complexes was conducted in 0.2% BSA HBS. The binding sensorgrams after single reference subtraction (biosensors with 0.2% BSA HBS, or Fab824, respectively) were used for global fitting. Because the response for FimH^{FocH} could be fit with a 1:1 Langmuir binding model, this model was employed for that data Prism 6 (GraphPad) software. Because the 1:1 model could not fit the FimH^{wt} data, the more complex conformational change model, used previously to model native, catch-bond forming FimH variants (32), was employed for that data using BIA evaluation (BIAcore). This is consistent with the notion that HAS is heavily predominant in FimH^{FocH}, while FimH^{wt} is in equilibrium between HAS and LAS conformations. The standard error that was given in the table for the kinetic parameters relates to the “fit” of the data to the model.

Protein Structure Analysis and Visualization

Protein Data Bank entries 4xo9 (chain A) and 4xo8 (chain A) (8) for crystal structures of FimH served as models of the LAS and HAS conformers. In addition, the NMR structure of isolated lectin domain (PDB entry 3zpd (chain A) (50)) was used to present the NMR-determined structural epitope of mAb824. Superposition of appropriate structures were carried out using the SUPERPOSE module (51) in the CCP4 program suite (52), and RMSDs and angle calculations were carried out using locally-written programs. Solvent-accessible surface area (SASA) of different forms of FimH lectin domains was calculated using the areaimol program within the CCP4 program suite (52). The SASA of functional and extended epitopes was calculated using PyMOL (DeLano Scientific LLC). The spatial distribution of amino acid residues involved in mAb epitopes and distances between Cα carbons were measured using PyMOL (DeLano Scientific LLC). The contact surface area between core-oriented side chains of V35 (in LAS) and L34 (in HAS) and their neighboring (in 3 Å distance) residues was determined using CHARMM (53). The contact surface area is defined by the difference between surface measured for all contacting side chains (defined in Figure S3) in the absence and the presence of the core-oriented side chain of V35 (or L34) (54,55).

TMD Simulation

The TMD simulation was started with the LD in LAS derived from the crystallographic structure with PDB code 3jwn (7). For reasons of efficiency, the pilin domain was removed and the lectin domain was truncated after residue 160 before starting the simulation. During the TMD run the crystallographic structure with PDB code 1uwf (56) was used to steer the protein conformation towards HAS. The simulation setup was similar as in a previous study (20). Briefly, the simulation was performed with the program NAMD (57) and the CHARMM22 force field (58). The protein was solvated using a cubic water box with side length of 86 Å and periodic boundary conditions. Chloride and sodium ions were added to neutralize the system and approximate a salt concentration of 150 mM. During the simulations, the temperature was kept constant at 300 K by using the Langevin thermostat (59) with a damping coefficient of 1 ps⁻¹, while the pressure was held constant at 1 atm by applying a pressure piston (60). Before starting the TMD run, a simulation was performed with LD in LAS for 10 ns without the addition of any forces. After this equilibration phase, forces were applied to the Cα atoms of regions of LD that were identified to significantly change conformation from LAS to HAS in a previous study (20), i.e., residues 22-26, 28-37, 58-69, 71-77, 109-124 and 150-157, and to the heavy atoms of residues that flip between solvent-exposed and core-buried orientations from LAS to HAS, i.e., residues 28, 33, 34, 35, 62, 63, 64, 67, 68, 118 and 119. Forces on these atoms were applied according to their RMSD from the target conformation (LD in HAS) following a TMD protocol (61). The TMD simulation was performed for 50 ns.

Time series of the side chain SASA values of switching residues were calculated using the program CHARMM (53) along a previously published MD simulation (20). This analysis included 25 non-glycine residues out of 29 total switching residues (defined in the study, see Figure S5). The correlation coefficient (R) between the time series values of the SASA (after averaging over a 240-ns time window) was calculated by performing a linear regression with the program xmgrace (plasma-gate.weizmann.ac.il/Grace/.

Structural Homology Analysis

For the structural homologues search, the conserved protein families' database Pfam (pfam.xfam.org/) was screened using the full-length protein sequence of FimH^{wt}. Resulting multiple sequence alignments retrieved from Pfam separately for lectin domain were examined for conservation of the allosteric switches (L34/V35 and Y64).

### Results

mAb824 Equally Recognizes Alternative Conformational States of FimH.

We compared binding of two monoclonal antibodies with two forms of FimH in purified fimbriae. The two antibodies are the previously characterized mAb21 and a novel mAb824, which is the focus of this study. One form of FimH was ‘wild-type’ FimH adhesin (FimH^{wt}) derived from E. coli K12 strain that is structurally identical to the FimH found commonly in uropathogenic E. coli, including a model cystitis strain J96. The other form was a recombinant FimH variant (FimH^{FocH}) that contains a 15-residue substitution in its PD that alters the interdomain interface, based on a different E. coli adhesin, FocH (9). In the absence of ligand, the LD of FimH^{wt} adopts the open-pocket LAS conformation that is not recognized by mAb21 (FIGS. 2A and 2C). Binding of α-methyl-D-mannopyranoside (αmm) causes the transition from LAS to HAS, thereby enabling mAb21 to recognize FimH^{wt} (FIGS. 2A and 2C). mAb21 binding to the αmm-induced HAS stabilizes the latter and, upon washing out αmm, enables FimH^{wt} fimbriae to strongly bind soluble horseradish peroxidase (HRP), a heavily mannosylated glycoprotein that binds very weakly to FimH^{wt} under static equilibrium conditions, i.e. the LAS conformation (FIG. 2D). Consistent with its disrupted LD/PD interface (9), the LD in FimH^{FocH} is predominantly in the HAS (FIG. 2B), based on previous studies showing that FimH^{FocH} has similar binding affinities as purified isolated LD, which is constitutively in HAS (9,10). As predicted, FimH^{FocH} fimbriae bind mAb21 equally well in the absence and presence of αmm and their binding to HRP is equally strong before or after forming a complex with mAb21 (FIGS. 2C and 2D).

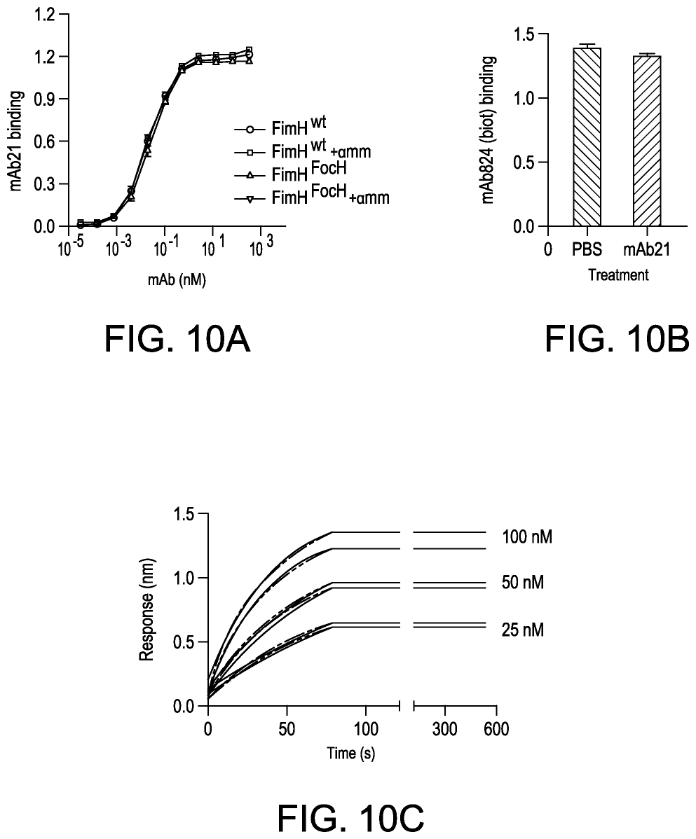

Unlike mAb21, mAb824 binds to both FimH^{wt} and FimH^{FocH} in the absence or presence of ligand (FIG. 2E and FIG. 10A), implying that its epitope is intact in both LD conformations and does not overlap with the mannose-binding pocket. Furthermore, mAb21 and mAb824 antibodies can bind to the HAS simultaneously (FIG. 10B), indicating that the two epitopes do not overlap. Together these observations imply that the mAb824 epitope is located somewhere between the ligand-binding pocket and the interdomain interface (FIG. 2A). The kinetics of mAb824 binding in the absence and presence of αmm are remarkably similar, with similar association rates (kₒₙ of 2.6±0.005 and 2.4±0.007×10⁵ M⁻¹s⁻¹, respectively) and immeasurably low dissociation rates (FIG. 10C). Altogether, the binding measurements are consistent with the novel mAb824 recognizing both the LAS and HAS conformations of FimH.

mAb824 Blocks the Allosteric Transition of FimH in Either Direction.

To assess if mAb824 binding to one conformation affects the ability to transition to the other, binding experiments were carried out on FimH^{wt} pre-treated with the Fab fragment of mAb824 (Fab824), when the LD is in the LAS or, alternatively, HAS conformation. Again, mAb21 binding was used as a probe for the HAS.

In the case of FimH^{wt} pre-treated with PBS only, increasing the αmm concentration generates mAb21 binding in a dose-dependent manner, with a plateau reached between 0.1-1.0% αmm (“PBS”, FIG. 2F). FimH pre-treated with Fab824 (presumably forming the LAS/mAb824 complex) shows significantly diminished binding to mAb21, requiring approximately three orders of magnitude higher αmm to exhibit a measurable mAb21 binding and, at 1% αmm, demonstrating only ˜30% of binding relatively to the PBS-treated sample. Thus, mAb824 appears to inhibit the LAS-to-HAS transition induced by αmm. Incubation of Fab824 with FimH^{wt} after the latter is activated by αmm (at 1% for 30 min) should produce an HAS/mAb824 complex. The pre-treatment with mAb824 in the presence of αmm (followed by a washout step to remove αmm) yields a species that binds mAb21 at ˜70% of maximal level even without αmm and shows no αmm-dependence of further activation (open triangles, FIG. 2G). This is in contrast to FimH^{wt} pre-incubated with αmm but without Fab824 that fully returned to LAS upon wash out of αmm (closed triangles, FIG. 2G). Taken together, the observations indicate that once bound to mAb824, the LAS/HAS transition of FimH^{wt} is strongly inhibited in both directions. In other words, mAb824 effectively traps FimH in the conformation to which it initially bound.

Blocking the Allosteric Transitions Inhibits Association of FimH with the Ligands.

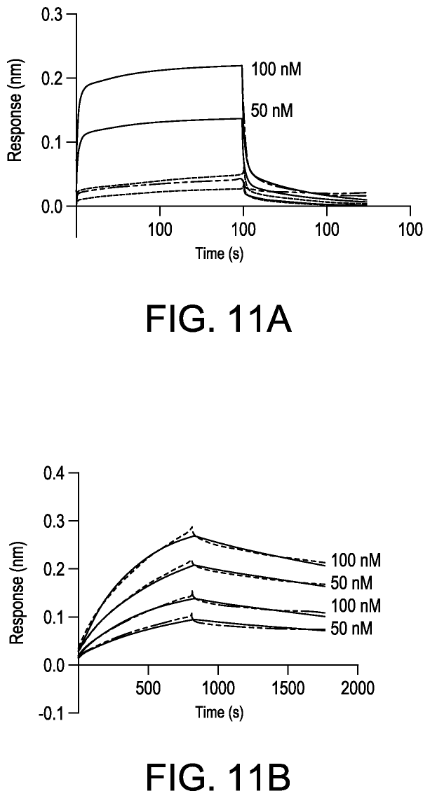

We tested whether blocking the allosteric transitions by mAb824 affects ligand binding kinetics of FimH^{wt} and FimH^{FocH}. Interaction of immobilized fimbriae with bovine RNase B, a model FimH ligand rich in N-linked high-mannose (Man₅) oligosaccharides, was measured by bio-layer interferometry (BLI) analysis. FimH^{wt} demonstrated nanomolar affinity (149±4 nM), with an association rate constant kₒₙ of 1.1×10⁶ M⁻¹s⁻¹ and a dissociation rate constant k_{off} of 0.26 s⁻¹ (Table 1 and FIG. 11A). Binding to FimH^{FocH} fimbriae occurred with ˜50-fold slower association rate but also ˜1000-fold slower dissociation rate compared to FimH^{wt}, for a ˜10-fold increase in binding affinity (Table 1 and FIGS. 11A and 11B). Pre-treatment of FimH^{wt} with Fab824 resulted in a large decrease in the RNase B-binding signal to a level as low as in the presence of saturating concentrations of soluble αmm. This decrease did not allow for reliable measurement of kₒₙ and k_{off} (FIG. 11A). Fab824 treatment of FimH^{FocH} resulted in a slight increase in the dissociation rate and more than 3-fold drop in the association rate, leading to a ˜5-fold decrease in the binding affinity based on K_{D} (Table 1 and FIG. 11B).

TABLE 1
Binding properties of FimH^{wt} and FimH^{FocH}.

| TEST | FimH variant | | PBS | αmm | mAb824^{*} |
|---|---|---|---|---|---|
| BLI^{‡} | FimH^{wt} | kₒₙ (M⁻¹s⁻¹) | 110 ± 3.2 × 10⁴ | Not | Not |
| | | k_{off} (s⁻¹) | 256 ± 6 × 10⁻³ | calculated | calculated |
| | | K_{act} (s⁻¹) | 7.2 ± 0.1 × 10⁻³ | | |
| | | k_{inact} (s⁻¹) | 12.8 ± 0.1 × 10⁻³ | | |
| | | K_{D} (nM)^{#} | 149 ± 4 | | |
| | FimH^{FocH} | kₒₙ^{*} (M⁻¹s⁻¹) | 2.2 ± .01 × 10⁴ | Not tested | 0.71 ± .002 × 10⁴ |
| | | k_{off}^{*} (s⁻¹) | 0.31 ± .001 × 10⁻³ | | 0.47 ± .001 × 10⁻³ |
| | | K_{D} (nM)^{**} | 13.9 ± .007 | | 66.6 ± .02 |

| | | | | |
|---|---|---|---|---|
| RBC agglutination^{†} | FimH^{wt} | 2 min | >60 min | >60 min |
| | FimH^{FocH} | 10 min | >60 min | >60 min |

^{*}Fab824 and mAb824 were used in the BLI and RBC agglutination test, respectively
^{†}Time (in min) upon which noticeable aggregation of RBC was observed
^{‡}Kinetic parameters for FimH^{wt} and FimH^{FocH} fimbriae binding to RNase B determined for BLI data presented in SI Appendix, Figure S2A and B. As described previously (32),
FimH^{wt} displays complex kinetics that require a conformational change model with four rate constants: the LAS association rate (kₒₙ), the LAS dissociation rate (k_{off}), rate of transition from LAS to HAS (‘activation’ rate; k_{act}), and rate of transition from HAS to LAS (‘inactivation’ rate; k_{inact}). In contrast, FocH displays simple kinetics that can be fit with a 1:1 binding model with two parameters for the HAS association rate (kₒₙ^{*}), and the HAS dissociation rate (k_{off}^{*}). The table shows the values calculated for data from one representative BLI experiment
^{#}K_{D} value was determined according to the conformational change model:
${K}_{D} = \frac{{k}_{off}}{{k}_{on}} ( \frac{{k}_{inact}}{{k}_{inact} + {k}_{act}} )$ ^{**}K_{D} value was determined according to the 1:1 binding model:
${K}_{D} = \frac{{k}_{off}^{*}}{{k}_{on}^{*}}$

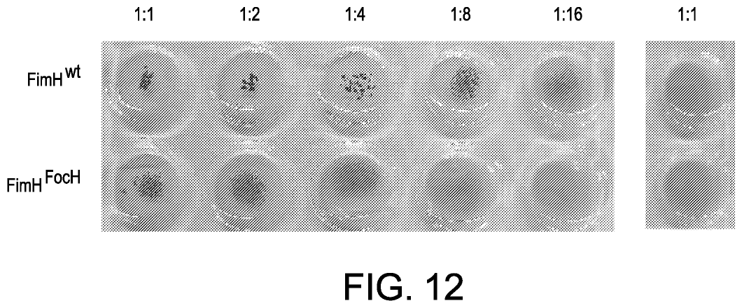

We also tested whether blocking the allosteric transitions affects cell-adhesive properties of FimH under shear conditions using the classical test of guinea pig red blood cell (RBC) aggregation by bacteria under dynamic rocking conditions. Bacteria expressing FimH^{wt}, which is able to shift from LAS to HAS under shear conditions, readily aggregates RBC within 2 minutes of rocking (Table 1). Bacteria expressing the HAS-trapped FimH^{FocH} required longer times and higher doses to aggregate RBC and the aggregates produced were smaller even at the highest bacteria dose (Table 1 and FIG. 12). These observations are consistent with the lower binding on-rate of the HAS conformation. Pretreatment with mAb824 completely abrogated aggregation caused by bacteria expressing either FimH^{wt} or FimH^{FocH} (Table 1).

In sum, the mAb824-rendered inability of the LAS to convert to the HAS yields an LD that is unable to associate even a highly mannosylated ligand at a measurable level. Furthermore, the on-rate of the antibody-trapped HAS is markedly reduced relative to the non-trapped FimH^{FocH}. Both effects could underlie the complete inhibition of bacterial binding to target cells under dynamic shear conditions observed here.

mAb824 Functional Epitope in LD is Conformationally Conserved in LAS and HAS.

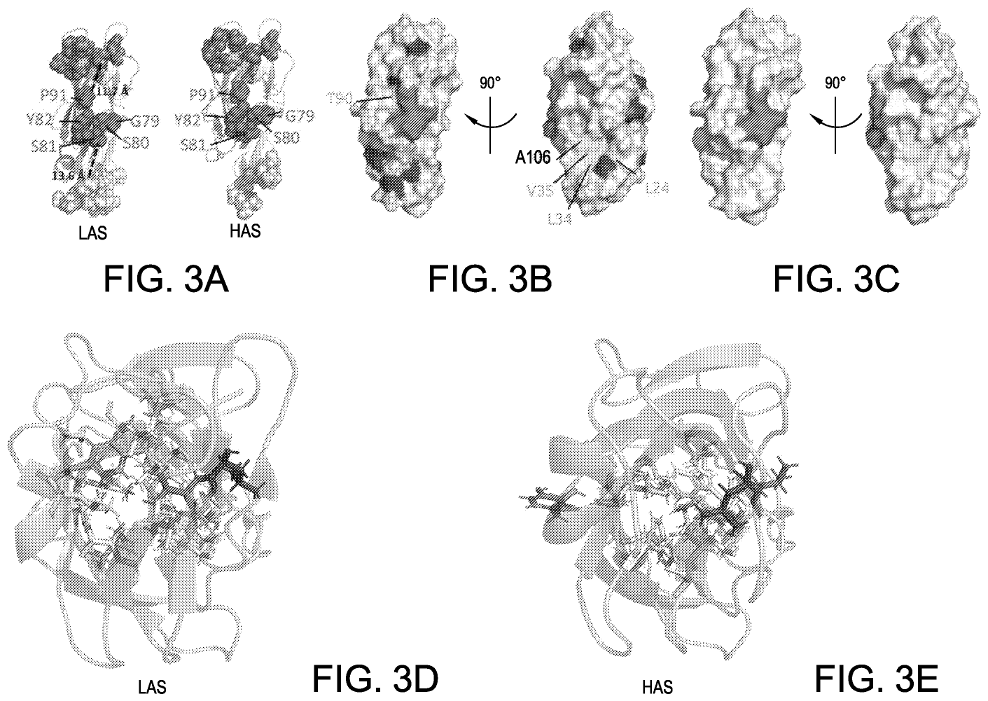

In an attempt to gain insight into the molecular mechanism of mAb824's effect on FimH, its epitope was mapped by mutagenesis. Analysis of a library 168 point mutations covering 83 LD positions revealed that substitution of S80 and Y82 with alanine eliminated or significantly decreased mAb824 binding to both FimH^{wt} and FimH^{FcoH} (Table 2), identifying those residues as energetically critical for the antibody binding and, therefore, part of the ‘functional epitope’ (14,15). Other substitutions that severely affected mAb824 binding were of G79, S81, and P91 with bulkier arginine. These residues are proximal to S80 and Y82 (FIG. 3A). Therefore, as predicted above (FIG. 2A), the mAb824 epitope is located between the mannose-binding pocket and LD-PD interface, with the shortest Cα distance from epitope residues (P91 and S81) in FimH to mannose-binding pocket (Q133) and interdomain interface (V30) residues is 11.7 Å and 14 Å, respectively (measured on PDB 4xo9) (FIG. 3A). Notably, all five mutagenesis-sensitive residues exist in similar structural positions in the two conformations: neither their solvent accessible surface area (SASA) (Table 3) nor their Cα positions (RMSD of 1.1±0.3 Å) differ substantially.

TABLE 2
The functional epitope of mAb824 as mapped by ELISA using FimH mutant library.

| | mAb824* | | | mAb824 | | | mAb824 | |
|---|---|---|---|---|---|---|---|---|
| Mutation | FimH^{wt} | FimH^{FocH} | Mutation | FimH^{wt} | FimH^{FocH} | Mutation | FimH^{wt} | FimH^{FocH} |
| F1L | 99 | 100 | T57R | 100 | 95 | T90N | 94 | 86 |
| A2S | 105 | NT | Q59A | 97 | NT | P91A | 93 | 84 |
| G8A | 103 | NT | R60P | 104 | 77 | P91R | 3 | 5 |
| T9G | 89 | NT | Y64F | 99 | NT | R92A | 100 | 105 |
| A10P | 102 | NT | Y64L | 90 | NT | R92D | 105 | NT |
| P12A | 108 | 101 | Y64E | 82 | NT | V93A | 100 | 82 |
| I13S | 99 | NT | Y64A | 81 | NT | P104L | 101 | 91 |
| N19C | 99 | NT | Y64R | 90 | NT | P104W | 101 | 101 |
| L24A | NT | 93 | V67K | 103 | 98 | A106V | 100 | NT |
| A25V | 101 | NT | L68V | 91 | NT | A106E | 90 | NT |
| P26G | 96 | 109 | S69C | 65 | NT | Y108E | 104 | NT |
| P26A | 92 | 93 | N70C | 88 | NT | P111K | NT | 101 |
| P26E | 99 | 96 | N70G | 92 | NT | S113C | 96 | NT |
| P26H | 90 | NT | F71A | 104 | NT | S114C | 95 | NT |
| V27A | 86 | 99 | S72A | 98 | 96 | G116C | 102 | NT |
| V27W | 105 | 90 | G73A | 100 | 104 | G117D | 85 | NT |
| V28T | NT | 94 | T74A | 92 | 88 | K121G | 104 | NT |
| N29C | 100 | NT | K76A | 113 | 99 | K121D | 93 | NT |
| V30C | NT | 109 | Y77A | NT | 100 | A122H | 104 | NT |
| N33L | 100 | NT | Y77N | NT | 92 | S124A | 98 | NT |
| N33K | 89 | NT | S78A | 105 | 98 | I130A | 92 | NT |
| L34A | 105 | NT | S78Y | 98 | 90 | R132D | 100 | 98 |
| L34E | 100 | NT | S78W | NT | 88 | R132H | NT | 106 |
| L34V | 103 | NT | G79A | 109 | 88 | Q133N | 89 | 94 |
| L34K | 94 | 98 | G79R | 4 | 6 | Q133A | NT | 97 |
| V35A | 109 | NT | S80A | 47 | 5 | T134G | NT | 98 |
| V35E | 88 | NT | S80R | 3 | 8 | N135I | 96 | 99 |
| V35F | 104 | NT | S80T | 106 | NT | N136A | NT | 89 |
| V35L | 105 | NT | S81A | 106 | 107 | Y137A | 103 | 99 |
| N46Q | 101 | 99 | S81G | 95 | 103 | N138I | 101 | 95 |
| N46A | 100 | NT | S81R | 96 | 14 | S139A | NT | 99 |
| D47S | NT | 97 | Y82A | 4 | 40 | F142A | 93 | NT |
| Y48A | 96 | NT | P83S | 97 | 100 | Q143A | 96 | 96 |
| E50A | 105 | 103 | P83R | 96 | 91 | V145I | 107 | NT |
| T51A | 107 | 98 | F84S | 96 | 80 | A150V | 103 | 98 |
| I52A | 101 | 98 | P85S | 98 | 109 | V155H | 84 | NT |
| T53A | 88 | NT | T87A | 106 | 95 | V155W | NT | 90 |
| T53R | 103 | NT | E89A | 65 | 94 | V156P | NT | 93 |
| D54E | NT | 81 | E89K | 99 | NT | | | |
| Y55A | NT | 98 | T90G | 99 | 98 | | | |

*Binding of mAb824 to purified isogenic fimbriae with different mutations in the lectin domain (LD) of FimH^{wt} and FimH^{FocH}. Relative binding (%) of the antibody as compared to not mutated LD of FimH^{FocH} is shown. The mutations reducing mAb binding >50% are marked in bold. NT, not tested.

TABLE 3
Solvent accessible surface area (SASA) of individual
amino acid resides in LAS to HAS states of FimH^{wt}.
SASA*

| FimH^{wt} | LAS | HAS | FimH^{wt} | LAS | HAS | FimH^{wt} | LAS | HAS |
|---|---|---|---|---|---|---|---|---|
| AA | (4xo9) | (4xo8) | AA | (4xo9) | (4xo8) | AA | (4xo9) | (4xo8) |
| PHE1 | 263 | 16 | ASP54 | 9 | 5 | LEU107 | 0 | 1 |
| ALA2 | 28 | 15 | TYR55 | 31 | 54 | TYR108 | 14 | 63 |
| CYS3 | 9 | 5 | VAL56 | 0 | 0 | LEU109 | 0 | 1 |
| LYS4 | 80 | 73 | THR57 | 14 | 6 | THR110 | 61 | 38 |
| THR5 | 12 | 7 | LEU58 | 0 | 0 | PRO111 | 53 | 10 |
| ALA6 | 57 | 68 | GLN59 | 30 | 45 | VAL112 | 109 | 42 |
| ASN7 | 126 | 115 | ARG60 | 144 | 114 | SER113 | 52 | 115 |
| GLY8 | 44 | 50 | GLY61 | 45 | 2 | SER114 | 139 | 102 |
| THR9 | 74 | 57 | SER62 | 0 | 34 | ALA115 | 96 | 10 |
| ALA10 | 75 | 67 | ALA63 | 23 | 9 | GLY116 | 29 | 38 |
| ILE11 | 12 | 2 | TYR64 | 13 | 88 | GLY117 | 49 | 27 |
| PRO12 | 96 | 80 | GLY65 | 5 | 44 | VAL118 | 28 | 77 |
| ILE13 | 64 | 119 | GLY66 | 42 | 13 | ALA119 | 69 | 3 |
| GLY14 | 25 | 34 | VAL67 | 86 | 6 | ILE120 | 1 | 1 |
| GLY15 | 76 | 17 | LEU68 | 0 | 105 | LYS121 | 123 | 141 |
| GLY16 | 54 | 33 | SER69 | 82 | 80 | ALA122 | 53 | 57 |
| SER17 | 78 | 79 | ASN70 | 147 | 35 | GLY123 | 44 | 54 |
| ALA18 | 6 | 6 | PHE71 | 37 | 16 | SER124 | 39 | 29 |
| ASN19 | 86 | 60 | SER72 | 61 | 63 | LEU125 | 43 | 48 |
| VAL20 | 3 | 3 | GLY73 | 11 | 18 | ILE126 | 0 | 2 |
| TYR21 | 106 | 119 | THR74 | 26 | 49 | ALA127 | 0 | 0 |
| VAL22 | 0 | 8 | VAL75 | 0 | 0 | VAL128 | 42 | 23 |
| ASN23 | 68 | 125 | LYS76 | 79 | 79 | LEU129 | 0 | 0 |
| LEU24 | 10 | 12 | TYR77 | 2 | 6 | ILE130 | 34 | 13 |
| ALA25 | 69 | 46 | SER78 | 63 | 73 | LEU131 | 0 | 0 |
| vPRO26 | 22 | 84 | GLY79 | 69 | 68 | ARG132 | 66 | 65 |
| VAL27 | 68 | 83 | SER80 | 52 | 53 | GLN133 | 44 | 6 |
| VAL28 | 103 | 3 | SER81 | 77 | 71 | THR134 | 37 | 32 |
| ASN29 | 71 | 61 | TYR82 | 55 | 60 | ASN135 | 27 | 23 |
| VAL30 | 148 | 79 | PRO83 | 75 | 73 | ASN136 | 83 | 86 |
| GLY31 | 64 | 73 | PHE84 | 4 | 10 | TYR137 | 127 | 134 |
| GLN32 | 43 | 88 | PRO85 | 55 | 61 | ASN138 | 62 | 69 |
| ASN33 | 5 | 94 | THR86 | 7 | 32 | SER139 | 108 | 126 |
| LEU34 | 26 | 1 | THR87 | 79 | 115 | ASP140 | 68 | 50 |
| VAL35 | 0 | 70 | SER88 | 64 | 69 | ASP141 | 64 | 78 |
| VAL36 | 0 | 5 | GLU89 | 70 | 76 | PHE142 | 133 | 51 |
| ASP37 | 55 | 59 | THR90 | 10 | 12 | GLN143 | 117 | 83 |
| LEU38 | 0 | 0 | PRO91 | 69 | 77 | PHE144 | 8 | 0 |
| SER39 | 31 | 36 | ARG92 | 133 | 162 | VAL145 | 43 | 34 |
| THR40 | 70 | 71 | VAL93 | 14 | 17 | TRP146 | 1 | 0 |
| GLN41 | 20 | 46 | VAL94 | 63 | 57 | ASN147 | 24 | 33 |
| ILE42 | 0 | 0 | TYR95 | 6 | 3 | ILE148 | 0 | 0 |
| PHE43 | 50 | 48 | ASN96 | 101 | 99 | TYR149 | 42 | 61 |
| CYS44 | 0 | 0 | SER97 | 36 | 45 | ALA150 | 1 | 4 |
| HIS45 | 36 | 37 | ARG98 | 125 | 115 | ASN151 | 77 | 76 |
| ASN46 | 2 | 2 | THR99 | 80 | 100 | ASN152 | 43 | 38 |
| ASP47 | 68 | 61 | ASP100 | 41 | 70 | ASP153 | 87 | 70 |
| TYR48 | 140 | 105 | LYS101 | 103 | 70 | VAL154 | 21 | 0 |
| PRO49 | 37 | 40 | PRO102 | 58 | 56 | VAL155 | 91 | 55 |
| GLU50 | 128 | 124 | TRP103 | 0 | 0 | VAL156 | 0 | 1 |
| THR51 | 103 | 71 | PRO104 | 37 | 33 | PRO157 | 25 | 52 |
| ILE52 | 32 | 22 | VAL105 | 2 | 0 | THR158 | 14 | 175 |
| THR53 | 28 | 38 | ALA106 | 7 | 11 | | | |

*The solvent accessible surface area (SASA) of amino acid residues in LAS and HAS conformers (PDB codes 4xo9 and 4xo8, respectively) were calculated individually by CCP4 software. The amino acid residues of the functional epitopes are marked in bold and the amino acid residues that showed large signal perturbation in NMR are shown in italics.

The mutagenesis identifies G79-Y82 and P91 as the functional epitope of the mAb824-LD interaction interface, with a combined SASA of 326 Å² (measured on PDB 4xo9). This area is much smaller than expected for a total antigen-antibody contact area (aka ‘structural epitope’) which generally expands beyond the binding-critical functional epitope and can include surface residues that are energetically not critical for antibody binding (14-16). Analyses of crystal structures of numerous antigen-antibody complexes reveal that such areas within a protein antigen are generally composed of 15-25 solvent-exposed residues and cover 1100±244 Å² on average and 600-650 Å² at a minimum (14-16).

NMR Spectroscopy Detects the mAb824-Perturbed Residues within the Putative Antibody Contact Area.

We used NMR spectroscopy on purified LD in complex with Fab824 to map the antibody contact area. At 69 kDa, the LD-Fab824 complex is not amenable to standard (¹H, ¹⁵N)-HSQC experiments, but methyl groups yield narrow line widths that make ¹³C-TROSY-HMQC spectra suitable for the complex (17). None of the residues identified in the functional epitope contain methyl groups, but FimH LD has 64 CH₃-containing residues (102 methyl groups total) that are well distributed throughout its structure. NMR assignments are available for 46 of the residues, providing good coverage throughout the LD. Approximately 55 methyl groups have some surface accessibility while others are buried.

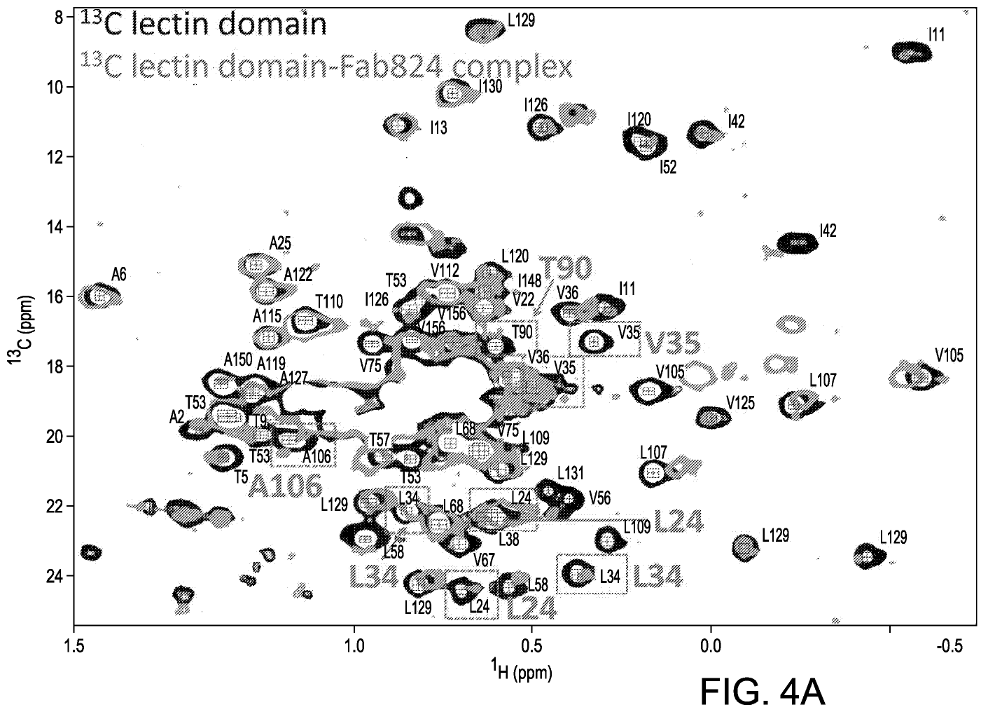

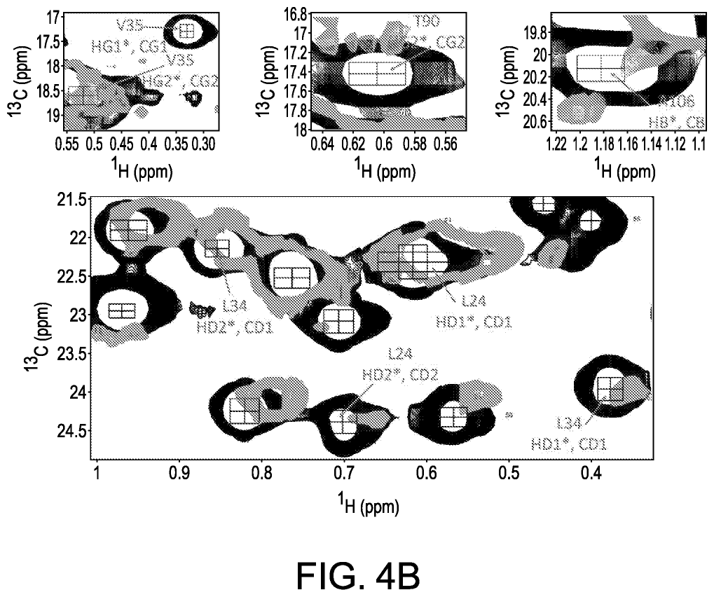

LD-Fab824 complex was purified by size exclusion chromatography from a mixture of uniformly ¹³C-labeled LD and natural abundance Fab824 in the absence of αmm. ¹³C-TROSY-HMQC spectra focused on methyl resonances were collected on LD unbound and bound to Fab824 (black and gray spectra, respectively, in FIG. 4A). Most resonances fully overlay between the two spectra, indicating there is no global conformational change upon Fab824 binding. This is consistent with the notion that the antibody traps LD in the conformation initially recognized. A subset of resonances, however, exhibits large perturbations indicative of changes in local chemical environment consistent with either a direct contact with antibody or an indirect conformational effect (18). The types of perturbations observed include: 1) no overlapping (gray) peak and 2) loss of intensity (small gray peak) (FIG. 4A and Table 4). Perturbations were observed for both burned and solvent-exposed methyl groups (expanded spectra of solvent-exposed residues are shown in FIG. 4B). We reasoned that large perturbations to buried methyl groups cannot be caused directly through antibody contact and must be affected indirectly (see Discussion) while NMR perturbations of solvent-exposed methyl groups could be due either to direct antibody contact, as would be expected if they are part of the antibody-binding contact surface, or to an indirect effect (18).

TABLE 4
The effect of mAb824 binding on methyl-containing residues of
FimH^{wt} lectin domain as determined by¹³C methyl-TROSY HMQC.

| Methyl Peaks | Assigned (A) | NMR observations: Large effects (L) |
|---|---|---|
| Residue # | Unassigned (U) | Small effect/Not perturbed (S) |
| A2 | A | S |
| T5 | A | S |
| A6 | A | S |
| T9 | A | S |
| A10 | A | S |
| I11 | A | L |
| I13 | A | S |
| A18 | A | S |
| V20 | U | |
| V22 | A | S |
| L24 | A | L |
| A25 | A | S |
| V27 | U | |
| V28 | U | |
| V30 | U | |
| L34 | A | L |
| V35 | A | L |
| V36 | A | L |
| L38 | A | L |
| T40 | U | |
| I42 | A | L |
| T51 | U | |
| I52 | A | L |
| T53 | A | S |
| V56 | A | L |
| T57 | A | S |
| L58 | A | L |
| A63 | U | |
| V67 | A | L |
| L68 | A | S |
| T74 | U | |
| V75 | A | L |
| T86 | U | |
| T87 | U | |
| T90 | A | L |
| V93 | A | |
| V94 | U | |
| T99 | A | |
| V105 | A | L |
| A106 | A | L |
| L107 | A | L |
| L109 | A | L |
| T110 | A | S |
| V112 | A | S |
| A115 | A | S |
| A118 | U | |
| A119 | A | S |
| I120 | A | L |
| A122 | A | S |
| L125 | A | L |
| I126 | A | L |
| A127 | A | S |
| V128 | U | |
| L129 | A | L |
| I130 | A | S |
| L131 | A | L |
| L134 | U | |
| V145 | U | |
| I148 | A | L |
| A150 | A | L |
| V154 | U | S |
| V155 | U | |
| V156 | A | S |
| T158 | U | |

Mapping the largely perturbed solvent-exposed residues onto the HAS LD structure (PDB 3zpd) reveals that a majority are either adjacent to the functional epitope (e.g., T90) or form a contiguous patch in proximity to it (L24, L34, V35, and A106) (FIG. 3B, lighter shading). Surface methyl groups that are near to perturbed residues but whose NMR resonances are not affected by antibody binding are shown with dark shading (FIG. 3B). Importantly, there are no unperturbed methyl-containing surface-exposed residues between the NMR-defined patch and the mutagenesis-sensitive patch. Thus, it is reasonable to conclude that the methyl-containing surface-exposed NMR-perturbed residues T90, L24, L34, V35, and A106 are within the contact area of mAb824. The most parsimonious conclusion is that the antibody-contact surface is composed of the functional epitope, the NMR-perturbed surface residues, and the intervening surface, for a total area of 854 Å² (as measured on PDB 3zpd), i.e. within the expected range of sizes of a structural epitope (FIG. 3C).

L34 and V35 Switch Between Buried and Exposed Orientations in the HAS and LAS.

Comparison of atomic-level structures of LAS and HAS of FimH^{wt} reveals that the NMR-perturbed surface-exposed residues T90, L24, and A106 have similar SASA values in both states (Table 3). In contrast, L34 and V35 are markedly different in their solvent exposure in the two states. V35 is buried in the LAS and exposed in the HAS (SASA 0 and 70 Å², respectively) (FIGS. 3D and 3E). In the LAS, the V35 side chain interacts with side chain atoms of seven other buried amino acids, with a total contact surface of 77 Å². Solvent-core dynamics of L34/V35 correlates with a solvent-core re-orientation of other core residues. An altered network of residues in 3 Å distance of core-buried V35 and L34 in LAS and HAS, respectively, is observed. The V35-interacting residues are: V36, Y64, L68, L107, L109, I126, V156, and L34-interacting residues are: L24, A25, V28, L109, I120, V154. The change in L34 results in its going from almost complete burial in the HAS (1 Å²) to solvent-exposed in the LAS (SASA 26 Å², partially shielded by side chains of neighboring solvent-exposed residues) (FIGS. 3D and 3E). Core-buried L34 in the HAS contacts side chains of six residues (total contact surface of 92 Å²), all but one of which are different from those that surround the V35 in its core-buried position. Taken together, these observations imply that the concomitant switching of L34 and V35 side chains between core and surface positions results in a distinct arrangement of the surrounding core residues.

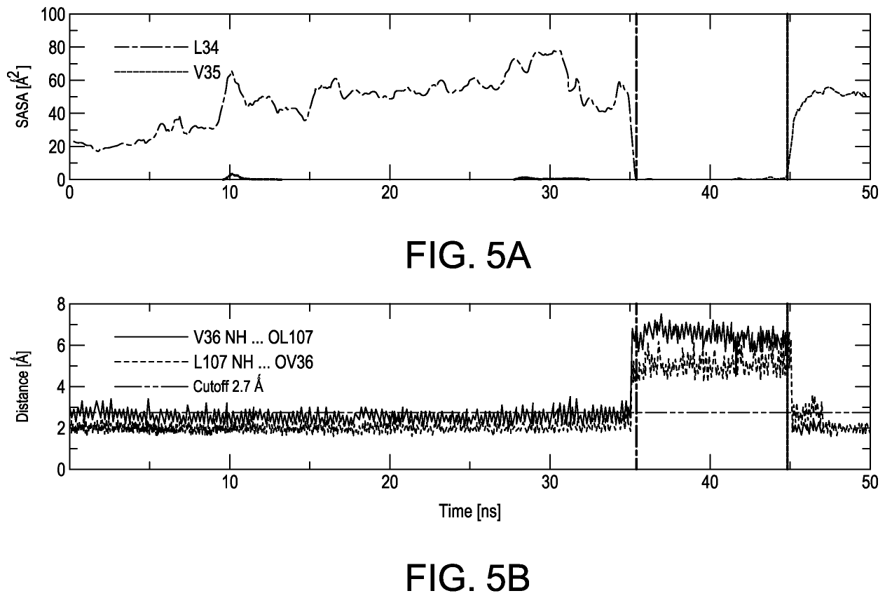

We performed a targeted molecular dynamics (TMD) simulation to predict conformational changes that may occur during the transition of the LD from the LAS to HAS (see Methods). The chain of events during the TMD simulations is depicted in a Supplemental movie file accessible through the online publication by Kisiela, D., et al. PLoS Pathog. 2021 Apr. 7; 17(4):e1009440. Concurrent with L34 switching from its surface-exposed orientation in LAS to its core-buried orientation in HAS (FIG. 5A), a rupture of hydrogen bonds between backbone atoms of the V35-neighboring V36 on one β-strand and L107 on the adjacent β-strand is predicted (FIG. 5A). Such an event is associated with formation of a 6 Å gap between the β-strands. This presumably high-energy “transition” state is followed by the core-to-surface switching of the V35 side chain (FIG. 56) and finally, resealing of the V36-L107 gap (FIG. 5B). Thus, the TMD simulation predicts that the switching of L34 and V35 between core and surface involves a high-energy transition state that is substantially different from either the LAS or HAS end-point structures.

Alanine Replacement of L34 or V35 Reduces mAb824's Ability to Block the Allosteric Transition.

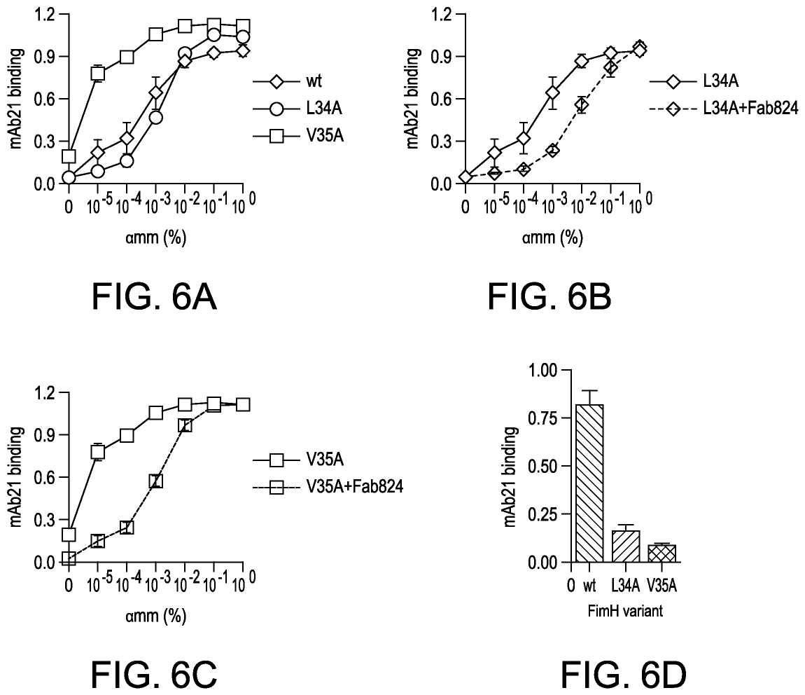

As shown in Table 2, replacement of L34 or V35 with a smaller hydrophobic alanine (or any other amino acid tested) does not affect mAb824 binding to FimH. We tested the effect of L34A and V35A substitutions on the ability of FimH to adopt LAS and HAS conformations. FimH^{L34A} fimbriae demonstrated FimH^{wt}-like ability to bind mAb21 without αmm or under increasing αmm concentrations (FIG. 6A), suggesting that substitution of L34 to alanine did not affect the mannose-dependent transition between LAS and HAS. FimH^{V35A} exhibited slightly increased binding of mAb21 in the absence of αmm and required a lower αmm concentration to fully adopt the HAS conformation, suggesting a modest shift in equilibrium away from the LAS and towards the HAS (FIG. 6A).

Having established that both alanine mutants retain the ability to transition between conformational states, we investigated whether LAS-HAS transitions in FimH^{L34A} and FimH^{V35A} are restrained by mAb824. Like FimH^{wt}, each variant binds to mAb824, but unlike the wild-type protein, the mAb824-bound mutants are still able to shift completely from LAS to HAS in the presence of αmm (FIGS. 6B and 6C, compare to FIG. 2F). Furthermore, mAb824 binding does not stabilize the αmm-induced HAS conformations of FimH^{V35A} or FimH^{L34A} because neither mutant was recognized by mAb21 when αmm is washed out of the mAb824-FimH complex (FIG. 6D). Thus, when either L34 or V35 is replaced with a less bulky alanine, the antibody's ability to block the transition between the conformational states is reduced.

Hydrophilic Replacement of V35 or L34 Stabilizes HAS or LAS, Respectively.

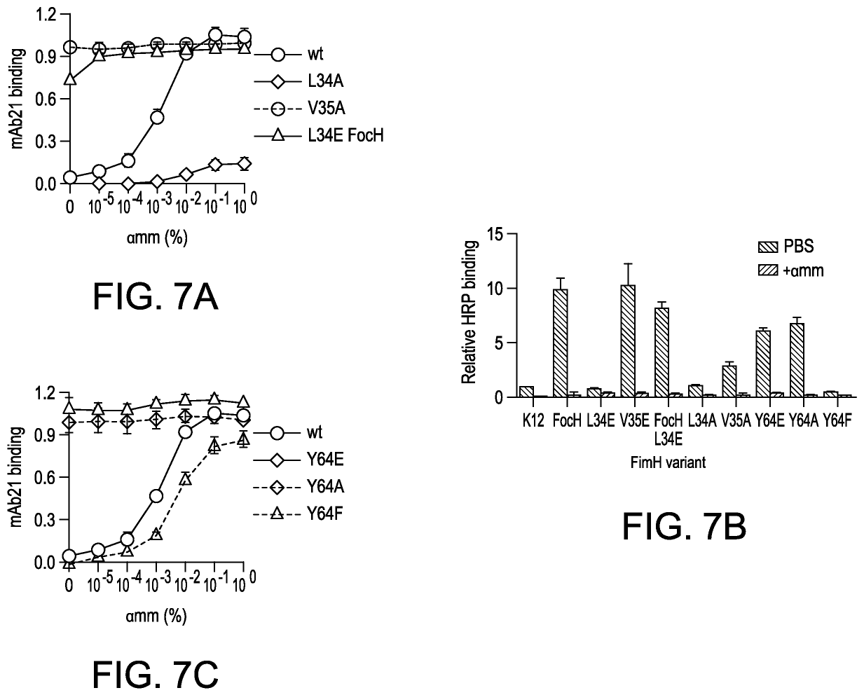

Unlike the alanine replacements, substitution of L34 and V35 with a charged glutamic acid resulted in profound changes. FimH^{V35E} is fully bound by mAb21 even in the absence of αmm (FIG. 7A), strongly resembling the phenotype of the FimH^{FocH} variant where the HAS conformation is predominant. In contrast, FimH^{L34E} was only marginally recognized by mAb21 even at the highest concentration of αmm tested (FIG. 7A). When the L34E LD was fused to the structurally altered PD (i.e., FimH^{FocH}), the mAb21 recognition was almost fully restored even in the absence of αmm, indicating that LD^{L34E} assumes a properly folded HAS when the interdomain interaction is disrupted. Thus, the absence of mAb21 binding to FimH^{L34E} cannot be attributed to misfolding or low expression, implying that the L34E replacement interferes with the LAS to HAS transition.

The effect of glutamic acid mutations just described correlates with the ability of immobilized fimbriae to bind soluble HRP (FIG. 7B). In FimH^{wt}, V35E bound HRP at least as well as FimH^{FocH}, while L34E exhibits marginal binding. In the FimH^{FocH} PD background, the L34E mutant binds HRP strongly, though not as strongly as FimH^{FocH} or FimH^{V35E}. As expected, FimH^{V35A} binding to HRP was only slightly enhanced relative to FimH^{wt}, while the L34A replacement did not affect the binding to a notable level.

Due to its negative charge and size, burial of a glutamic acid side chain is disfavored, especially relative to valine or leucine. Our results indicate that replacing either L34 or V35 with a Glu stabilizes the LAS and HAS of FimH, respectively, and in a manner that allows the Glu side chain to remain on the surface and avoid burial. Therefore, the orientations of L34 and V35 are critical for maintaining the LD in either the LAS or the HAS. We conclude that these two residues within the mAb824 contact area act as conformational ‘toggle switches.’

L34/V35-Like Switching is Common and Occurs in a Concerted Fashion.

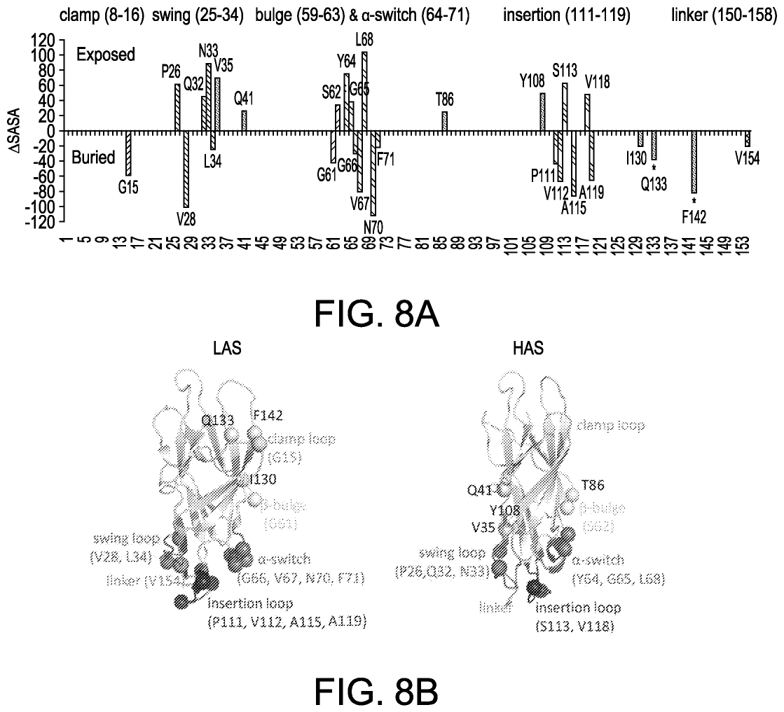

Although the total SASA of LD in the LAS (including the interdomain interface) and HAS are nearly identical (7559 and 7460 A², respectively), the identity of residues that are core-buried or surface-exposed differs in the two states. Twenty-nine LD residues experience a significant change in SASA (both >2-fold and >20 Å²) between the conformations (FIG. 7A). Thirteen residues are more solvent-exposed and 16 residues are less solvent-exposed in the HAS compared to the LAS. Nearly half of these residues (13 out of 29) are aliphatic or aromatic and most are in regions identified to undergo conformational changes, namely, the swing loop, insertion loop, β-bulge and α-switch, as well as the mannose-binding pocket (FIGS. 8A and 8B) (7,19). Overall, a significant change in SASA values is observed in about a quarter of residues with low solvent exposure (SASA <20 Å²) in LAS or HAS (Table 3).

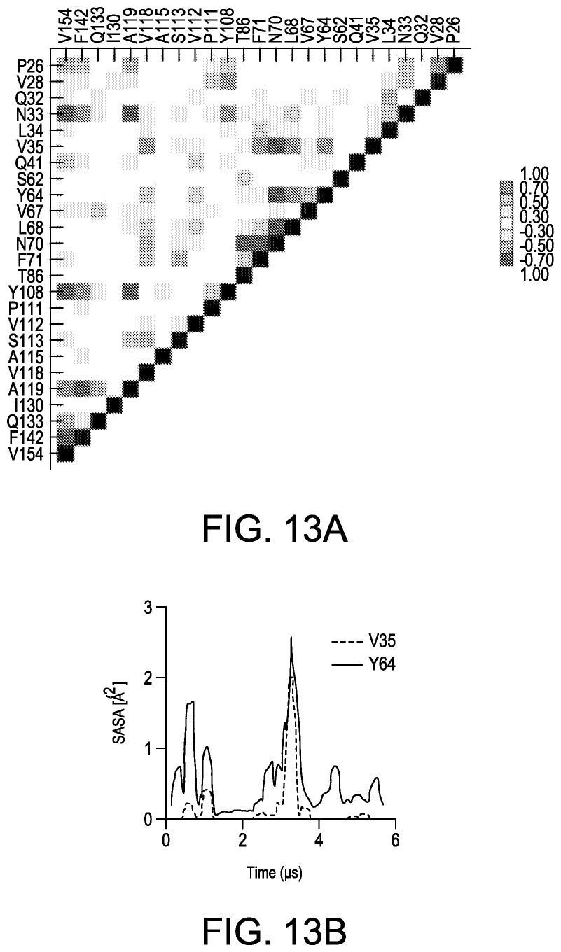

Examination of side chain SASA values for the residues with large differences in the two states along the 5.8-ρs long MD simulation from an earlier study (20) revealed that 14 of the residues have a strong association in the timing of their solvent exposure with at least one other residue, with the Pearson's correlation coefficient R being either above 0.7 or below −0.7 (FIG. 13A). Eleven residue pairs exhibit a positive relationship, i.e. their SASA values co-varied in time in the same direction, while another eleven pairs exhibit a strong negative relationship with the SASA values changing together in time but in opposite directions. Notably, the SASA changes of V35 correlate positively with four residues—Y64, L68, F71, and V118—more than any other switching residue.

Altogether the observations indicate that, in addition to L34 and V35, a sizable proportion of LD residues undergoes significant changes in orientation between LAS and HAS. The implication is that the FimH conformational transition is associated with a major repacking of side chains within the core of the LD. Moreover, the strong correlation in the timing of changes suggests that those changes occur in a concerted fashion.

Y64 Also Functions as a Toggle Switch.

We studied one of residues that closely follows the switching pattern of V35 in more detail. Tyrosine-64 is core-buried in the LAS (SASA 13 Å²) and solvent-exposed in the HAS (SASA 88 Å²) (FIGS. 3D and 3E). In LAS, Y64 and V35 side chains are in close contact and the timing of their switch out of the core is positively correlated with R=0.75 (FIG. 13B). In its solvent-facing orientation in HAS. Y64 is on the opposite face from the putative mAb824 contact area and is 17 Å from the closest residue of the functional epitope.

Similar to the V35E mutation, Y64E adopts the HAS conformation even in the absence of αmm (FIG. 7C), indicating that the core orientation of Y64 is important for LAS stability. Substitution of Y64 with alanine also shifts FimH to the HAS (FIG. 7C). Replacement of the tyrosine to a structurally similar but more hydrophobic phenylalanine resulted in FimH that requires slightly higher αmm concentrations than FimH^{wt} to adopt the HAS (FIG. 7C). This is consistent with the more hydrophobic phenylalanine ring favoring the LAS (where it will be buried) relative to the HAS (where it will be surface-exposed). The conformational states of the Y64 mutants as detected by mAb21 fully correlate with their HRP-binding capability, with FimH^{Y64E} and FimH^{Y64A} exhibiting strong HRP binding and FimH^{Y64F} showing reduced binding (FIG. 7B). Altogether, the results identify Y64 as another SASA-switching residue that functions as a toggle switch critical for the allosteric transition of FimH.

The Allosteric Toggle Switches are Conserved Across a Broad Range of Fimbrial Adhesins.

To explore whether other bacterial adhesins might have FimH-like allosteric switches, we analyzed proteins deposited in the Pfam database (21). The analysis identified over two dozen representative proteins with structural homology to the FimH LD with a broad range of primary structure identity—from 88% to 17% (Table 5 and, FIG. 14). All FimH homologues were designated as known or putative fimbrial adhesins found in various enterobacterial species, including common human pathogens like E. coli, Klebsiella, Proteus, Citrobacter and Serratia. However, only two proteins in the list have a known ligand specificity—the mannose-binding adhesin, FimH, from Klebsiella pneumoniae (accession #A6TDM0, 88% identity to E. coli FimH) and galactose-binding adhesin, FmIH, from E. coli (accession #P77588, 41% identity to E. coli FimH). None of the adhesin proteins has been reported to be allosteric, i.e., able to shift between functional states through action at a distance.

TABLE 5
Pfam database-retrieved structural homologues of E. coli FimH adhesin.

| | Accession | | Identity | Receptor | Protein |
|---|---|---|---|---|---|
| No. | number* | Species | (%)† | specificity | Length (AA) |
| 1 | P08191 | Escherichia coli | query | α-D-mannose | 300 |
| 2 | A6TDM0 | Klebsiella pneumoniae | 88 | α-D-mannose | 301 |
| 3 | A0A2X3GE33 | Kluyvera cryocrescens | 84 | | 301 |
| 4 | A0A0L0AF88 | Klebsiella sp. | 81 | | 301 |
| 5 | B2VEY7 | Erwinia tasmaniensis | 67 | | 304 |
| 6 | U2MA57 | Serratia fonticola | 66 | | 305 |
| 7 | A0A1I4X1B9 | Izhakiella capsodis | 66 | | 313 |
| 8 | A0A0J8YTF1 | bacteria symbiont BFo2 of | 64 | | 301 |
| | | Frankliniella occidentalis | | | |
| 9 | A0A085JND3 | Tatumella ptyseos | 63 | | 187 |
| 10 | W0LFI5 | Chania multitudinisentens | 63 | | 305 |
| 11 | W0L8D6 | Chania multitudinisentens | 54 | | 311 |
| 12 | A0A1B7JTX7 | Providencia heimbachae | 52 | | 312 |
| 13 | A0A0J5NKQ8 | Pluralibacter gergoviae | 46 | | 302 |
| 14 | A0A336Q1W3 | Citrobacter koseri | 43 | | 300 |
| 15 | B4F2K7 | Proteus mirabilis | 42 | | 310 |
| 16 | A0A095UYJ3 | Tatumella morbirosei | 41 | | 299 |
| 17 | P77588 | Escherichia coli | 41 | Gal (β1-3) | 304 |
| | | | | GalNac | |
| 18 | A0A085JP12 | Tatumella ptyseos | 41 | | 191 |
| 19 | B4ETK5 | Proteus mirabilis | 34 | | 303 |
| 20 | A0A348HGN6 | Zymobacter palmae | 28 | | 304 |
| 21 | A0A348HGN5 | Zymobacter palmae | 28 | | 337 |
| 22 | A0A1E7Z0K3 | Candidatus Erwinia dacicola | 27 | | 309 |
| 23 | A0A0J8YKK0 | bacteria symbiont BFo1 of | 26 | | 311 |
| | | Frankliniella occidentalis | | | |
| 24 | K8WLZ7 | Providencia sneebia | 25 | | 333 |
| 25 | A0A0L7THN9 | Erwinia iniecta | 24 | | 326 |
| 26 | B2VGL4 | Erwinia tasmaniensis | 24 | | 323 |
| 27 | D4DZU5 | Serratia odorifera | 24 | | 310 |
| 28 | A0A2G8E182 | Erwinia sp. | 19 | | 264 |
| 29 | A0A495RCD0 | Orbus hercynius | 18 | | 321 |

*Selected structural homologues of FimH lectin domain as determined by Pfam database search using the sequence of FimH^{wt} (P081191) as query.
†identity (%) of lectin domain sequence (1-160 AA) is shown.

Sequence alignment revealed that positions around L34/V35 and Y64 are highly conserved (FIG. 14). The corresponding amino acid homologues could be identified with certainty in proteins with as low as 34% identity to FimH. Positions homologous to L34 and V35 were identical to FimH or contain a structurally similar isoleucine. The Y64 homologous position is either tyrosine, phenylalanine or, in one case, leucine.

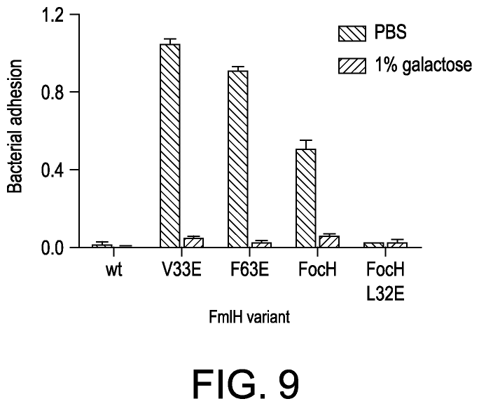

To test whether amino acids homologous to FimH L34, V35, and Y64 might function as allosteric switches, we focused on FmIH, where L32, V33, and F63 are the predicted homologues, respectively (FIG. 9A). FmIH is known to bind to terminal galactose (β1-3) N-acetylgalactosamine, although with low affinity (22). When full-length wild-type FmIH (FmIH^{wt}) was expressed in type 1 fimbrial background, no bacterial binding was detected to asialated fetuin, which contains oligosaccharides with galactose (β1-3), even after spinning the bacteria down to the asialofetuin-coated surface (FIG. 9B). Remarkably, substitution of glutamic acid for either V33 or F63 yielded mutants with a highly pronounced and galactose-inhibitable binding phenotype (FIG. 9B). An L32E mutation did not increase binding. To determine whether L32E inhibits the binding properties of FmIH, its lectin domain was fused to the heterogeneous pilin domain used for the construction of HAS-favoring FimH^{FocH} variant. Like FimH^{FocH}, the FmIH^{FocH} variant containing wild-type FmIH LD exhibited increased binding relative to the wild-type adhesin (FIG. 9B). In this context, introduction of the L32E mutation decreased the binding properties of FmIH^{FocH} in an even more pronounced manner than L34E in FimH^{FocH} (FIG. 7B). Thus, V33E and F63E mutations resulted in activation of the binding phenotype in FmIH, while L32E had the opposite effect, equivalent to phenotypes observed for the corresponding amino acid replacements in homologous positions in FimH.

### Discussion

This Example shows that 1) a monoclonal antibody that binds both HAS (‘active’) and LAS (‘inactive’) conformations of the FimH adhesin blocks the allosteric transition between the states in both directions, 2) restraint of the conformational transition results in elimination of bacterial adhesion under dynamic shear conditions, 3) the antibody-FimH contact area includes toggle-switch-like residues that must transit between core-buried and solvent-exposed orientations during the transitions, and 4) similar allosteric toggle switches are found in other FimH regions and across functionally and structurally diverse fimbrial adhesins of enterobacteria.

In contrast to previously discovered antibodies against allosteric proteins that recognize only one conformational state (23-29), the mAb824 antibody described here binds both and traps the protein in the state to which it initially binds. The binding-critical, functional epitope of mAb824 is structurally similar in both conformations, providing a rationale for how both conformations are recognized by mAb824, but not for how it can trap each state. Antibody-antigen contact areas (i.e., structural epitope) are known to extend well beyond the residues that are energetically critical for binding (14,30,31). Precise mapping of antibody-antigen interfaces requires atomic-level structures, but these remain challenging to obtain (14). We used methyl group-specific NMR and identified a region on the LD surface that is close to the functional epitope and exhibits substantial spectral perturbation upon mAb824 binding to FimH. We assign this region as the contact area for mAb824. The proposed antibody-LD contact area does not overlap with the mannose-binding pocket, consistent with the observation that αmm does not inhibit mAb824 binding to FimH even at the highest concentrations tested.

Unlike the functional epitope which is retained in the two conformations, the broader mAb824-LD contact area includes residues whose orientations switch between core-buried and surface-exposed, namely L34 and V35. Replacement of either of these aliphatic residues with a charged glutamate revealed that the surface-exposed orientations of L34 and V35 are critical to the LAS and HAS states, respectively. It has been shown that there is little free space between antibody and protein antigen atoms in antibody-antigen contact areas, suggesting that longer and/or bulkier side chains of epitope surface residues lie relatively flat (14). This property implies that antibody binding is likely to restrict the side chains of L34 and V35 on the surface and hinder their switching in and out the core. Such a restriction would in turn block the shift from LAS to HAS and vice versa. In addition, TMD simulations predict that the L34/V35 switching requires transient formation of a large (˜6 Å) gap between neighboring β-strands that would represent a substantial perturbation in structure that may also be restrained by mAb824 binding. Importantly, when a smaller alanine sidechain is introduced at either position 34 or 35, mAb824 does not block the transition as effectively. Composed of a single methyl group, surface-core switching of alanine is likely less restrained. The alanine results support our hypothesis that mAb824 binding to FimH inhibits both LAS->HAS and HAS->LAS conformational transitions by providing a steric block to reorientation of residues between their core-buried and surface-exposed orientations. This in turn reveals a molecular mechanism for the inhibition of allosteric transitions by mAb824 that has not been previously described for an antibody-antigen interaction to our knowledge.

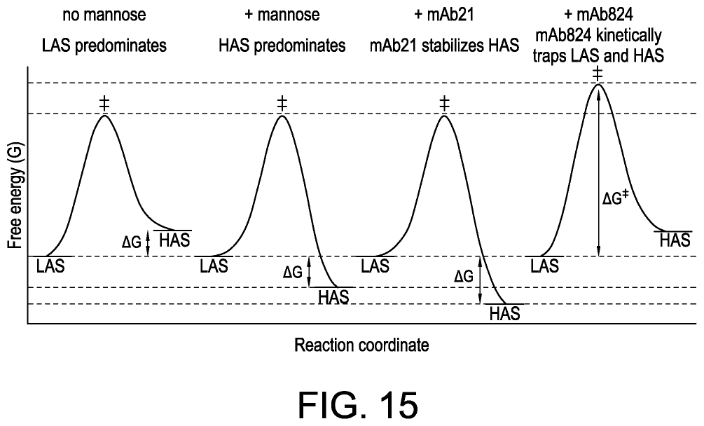

Taken together, the results suggest that mAb824 acts as a kinetic trap that blocks the conformational transitions in either direction rather than a thermodynamic sink that stabilizes a specific conformation as in the case of allosteric antibodies. In this new paradigm, mAb824 binding increases the free energy of the transition state between LAS and HAS rather than lowering the free energies of the end-state conformations (FIG. 15). Because the mannose binding and domain separation are reciprocal allosteric events, one facilitating another, the mannose- or tensile force-induced domain separation and, thus, LAS to HAS transition is likely to involve same molecular mechanism of allosteric propagation through the LD, either from the binding pocket to the interdomain interface or in the opposite direction. In turn, the kinetic trapping mechanism is likely to occur under either equilibrium or dynamic conditions, as supported by the functional studies below.

The novel kinetic trapping mechanism provided an opportunity to compare functional properties of the alternate conformations of FimH when they are unable to exchange. We tested the interaction of FimH^{wt} and FimH^{FocH} fimbriae with RNAseB and found that kₒₙ is ˜50-fold higher and k_{off} is nearly 1000-fold higher for FimH^{wt} relative to FimH^{FocH}, resulting in ˜10-fold lower affinity. It has previously been estimated that the affinity of LAS towards mannose ligands is ˜300-3,000-fold lower than that of HAS (8,9,11,32). RNAse B's abundant Man₅ oligosaccharide moieties are likely the source of the smaller difference in affinity for RNAseB between native FimH and FimH that is trapped in HAS. Among natural ligands, Man₅ has the highest affinity to FimH LAS conformers, likely due to the terminal oligomannose in the moiety Man(α1-3)Man(β1-4)(GlcNAc)₂-Asn that interacts with LD via a structurally extended binding site (33). The larger affinity differences previously reported were based on measurements made with either mannose derivatives or mannosylated oligosaccharides that lack the specific terminal oligomannose component. Those ligands interact with FimH via the mono-mannose mechanism that results in strong binding for FimH in the HAS, but not in the LAS conformation, in contrast to the oligo-mannose (also called ‘tri-mannose’) binding mechanism (9,10).

Despite the N-linked Man₅ oligosaccharide moiety that binds strongly to all natural FimH variants, RNAse B did not bind at detectable levels to the mAb824-trapped LAS conformation of FimH^{wt}. As mAb824 does not directly block the mannose-binding pocket, its inhibitory effect must be due to disabling the transition from the LAS to HAS. Hence, even when binding to its strongest natural ligands, FimH must convert from the wide-pocket LAS conformation to a narrow-pocket conformation observed in either the HAS or the intermediate state of FimH. Indeed, ligand binding to the HAS conformation of FimH occurs at a substantially reduced on-rate relative to native FimH (8,32). While mAb824 does not fully block RNAse B binding to HAS-favoring FimH^{FocH}, it slowed the on-rate for ligand binding even further. The most parsimonious explanation for these observations is that the ligand-binding pocket visits both closed and open conformations in either state and the frequency with which this occurs determines the on-rate. Thus, while FimH that is trapped in the HAS conformation can bind ligand, it can do so only under conditions that allow ample time for ligand entrance into the binding pocket.

Guinea pig RBC aggregation is the classical test for type 1 fimbriated bacteria binding under dynamic shear conditions. The surface of these cells is rich in mannosylated glycoproteins and aggregation is induced under continuous rocking of the bacterial and RBC suspension. Under those conditions, FimH acts as a catch-bond adhesin: it binds rapidly to cell surface mannose and then strengthens its binding to resist detachment under tensile force. In the ‘kinetic selection’ mechanism for such binding, the LAS conformation initiates ligand binding and because it has an open binding pocket, the on-rates are very fast (32). Sustained binding requires transition to the HAS with its narrow pocket and slow dissociation. Tensile force favors this transition by inducing domain separation of the LD and PD and is only applied once FimH is bound to ligand, so this mechanism effectively accomplishes the catch bond function of FimH. mAb824's ability to completely inhibit bacteria expressing FimH^{wt} to aggregate RBCs confirms the validity of the kinetic selection model that allosteric conformational shifts are critical for FimH-mannose binding under dynamic conditions. mAb824 represents a novel type of highly effective adhesion-inhibiting antibody that does not act by direct obstruction of the binding pocket but rather by disabling the catch bond properties of FimH.

The mechanism by which L34 and V35 control the conformational, and thus, functional states of FimH through reorientation of their side chains in and out of the protein core is analogous to the action of toggle switches, in which two alternating positions define the on/off status of a device. Alternative conformation-specific orientations of side chains have been reported in other allosteric proteins, e.g. periplasmic (maltose) binding protein MaIE (34), rhodopsin (35), integrin (36), DNA-binding receptor NtrC (37), and anti-thrombin III (38). However, the consequences of the toggle switch-like effect on the conformation and function of allosteric proteins has not been studied thoroughly, nor are they fully appreciated. Our successful activation or de-activation of FimH (and also FmIH adhesin as discussed below) by glutamate mutations at L34/V35 suggests that replacement of hydrophobic residues well removed from ligand-binding pockets with charged residues can serve as a strategy to identify putative toggle switches and discover alternate functional states (“E-scanning” in the case of glutamate substitutions). In support of the E-scanning approach, glutamate replacement of FimH surface-core switching residue Y64 produces a “trapped” HAS conformation, while substitution with phenylalanine, which is slightly more hydrophobic, results in a slightly more stable LAS. These results confirm the importance of specific sidechain orientation for the conformational transition.

The importance of core rearrangements in the FimH allosteric transition is supported by the observation that dozens of residues in the LD switch between primarily core-buried and solvent-exposed orientations in LAS and HAS, nearly half of which are aliphatic or aromatic. The sheer number of residues involved implies that core repacking occurs during the allosteric transition, consistent with the notion that the structural architecture and dynamics of the hydrophobic core play a vital role in the allostery of proteins (39-41). Given the closely packed nature of protein cores, it is reasonable that repacking occurs in a concerted fashion. Consistent with this notion, we observe that the timings of the orientational change of many core residues exhibit strong positive or negative correlation with each other in simulations. This suggests that during the allosteric transition in FimH, a change in one residue depends on and causes change in other residue(s) or, alternatively, changes in multiple residues co-depend on a single trigger event. If critical residues are hindered from adopting their new positions during the conformational change, the entire transition may be effectively blocked. We believe that the toggle switch residues represent such positions.

Binding of mAb824 to a surface region of FimH LD that contains toggle switch residues causes perturbations to core-buried residues, as revealed by our NMR analysis. While the nature of these perturbations remains to be determined, many of the perturbed core-buried side chains are in direct contact or proximal to the core-surface switches. The observed NMR spectral broadening is consistent with a change in the internal dynamics upon antibody binding.

Finally, the broad applicability of the allosteric toggle switches and E-scanning concept is supported by our analysis of other fimbrial adhesins. While hundreds of known or putative bacterial adhesins have been characterized and some were shown to mediate catch bond-like adhesion (42,43), none have been shown to be allosteric or to shift between alternate functional states besides E. coli FimH. Pfam database analysis suggested that enterobacterial adhesins with as little as 17% sequence identity to FimH are structurally homologous to it. Enterobacteria is the largest and most diverse group of Gram-negative bacterial pathogens causing a variety of infections in humans, with the fimbrial adhesins being among the major virulence factors (44,45). The L34/V35 and Y64 positions are highly conserved across FimH homologues, suggesting that the allosteric toggle switch function of the homologous amino acids may be preserved. We confirmed this prediction for one of the few FimH homologues with a known binding ligand—the galactose-binding adhesin, FmIH, positioned on the tip of F9 fimbriae that are implicated in the urovirulence of E. coli (22). Glutamate substitution of the three putative toggle switch residues can both activate and inactivate the galactose-specific binding of FmIH. These results strongly suggest that FmIH has HAS and LAS conformations and, thus, may mediate adhesion via the allosteric catch bond mechanism (currently under investigation). We propose that a broad range of bacterial and non-bacterial adhesion/attachment proteins are allosteric and use similar types of molecular toggle switches to transit between LAS and HAS states. Moreover, the E-scanning technique could be used to induce the HAS, strongly ligand-binding state in allosteric proteins to enable their detailed characterization or even to identify ligands.

In summary, this Example provides deeper mechanistic insights into the molecular mechanism and kinetics of allosteric transitions in FimH, and potentially, in a broad range of cell adhesion proteins. We have defined experimental approaches to trap the LAS and HAS conformations of an allosteric protein by a novel type of antibody and to induce their specific functional states by specific types of mutational alterations in the search for allosteric toggle switch residues.

### REFERENCES

1. Lebon G, et al. Nature. 2011 May 18; 474(7352):521-5.
2. Melcher K, et al. Nature. 2009 Dec. 3; 462(7273):602-8.
3. Evenäs J, et al. J Mol Biol. 2001 Jun. 15; 309(4):961-74.
4. Johnson J R. Clin Microbiol Rev. 1991 January; 4(1):80-128.
5. Jones C H, et al. Proc Natl Acad Sci USA. 1995 Mar. 14; 92(6):2081-5.
6. Choudhury D, et al. Science. 1999 Aug. 13; 285(5430):1061-6.
7. Le Trong I, et al. Cell. 2010 May 14; 141(4):645-55.
8. Sauer M M, et al. Nat Commun. 2016 Mar. 7; 7:10738.
9. Aprikian P. et al. J Biol Chem. 2007 Aug. 10; 282(32):23437-46.
10. Tchesnokova V, et al. J Biol Chem. 2008 Mar. 21; 283(12):7823-33.
11. Sauer M M, et al. J Am Chem Soc. 2019 Jan. 16; 141(2):936-44.
12. Thomas W. et al. Biophys J. 2006 Feb. 1; 90(3):753-64.
13. Tchesnokova V, et al. Infect Immun. 2011 October; 79(10):3895-904.
14. Ramaraj T, et al. Biochim Biophys Acta. 2012 March; 1824(3):520-32.
15. Sivalingam G N, et al. Mol Immunol. 2012 July; 51(3-4):304-9.
16. Stave J W, et al. J Immunol Baltim Md 1950. 2013 Aug. 1; 191(3):1428-35.
17. Wiesner S, et al. Curr Opin Struct Biol. 2015 December; 35:60-7.
18. Simonelli L, et al. Methods Mol Biol Clifton NJ. 2018; 1785:29-51.
19. Rodriguez V B, et al. J Biol Chem. 2013 Aug. 16; 288(33):24128-39.
20. Interlandi G, et al. Proteins. 2016 July; 84(7):990-1008.
21. El-Gebali S, et al. Nucleic Acids Res. 2019 Jan. 8; 47(D1):D427-32.
22. Kalas V, et al. Proc Natl Acad Sci USA. 2018 Mar. 20; 115(12):E2819-28.
23. Byron A, et al. J Cell Sci. 2009 Nov. 15; 122(Pt 22):4009-11.
24. Luo B-H, Benjamin D C, Perdue S S. J Biol Chem. 2004 Jun. 25; 279(26):27486-71.
25. Mould A P, Benjamin D C, Perdue S S. J Biol Chem. 1996 Aug. 23; 271(34):20365-74.
26. Mould A P, Benjamin D C, Perdue S S. Biochem Soc Trans. 1995 August; 23(3):395S.
27. Oyen D, Benjamin D C, Perdue S S. J Mol Biol. 2011 Mar. 18; 407(1):138-48.
28. Paduch M, Benjamin D C, Perdue S S. Methods 2013 Mar. 15; 60(1):3-14.
29. Rizk S S, Benjamin D C, Perdue S S. Nat Struct Mol Biol. 2011 April; 18(4):437-42.
30. Cunningham B C, Wells J A. J Mol Biol. 1993 Dec. 5; 234(3):554-63.
31. Benjamin D C, Perdue S S. Methods 1996 June; 9(3):508-15.
32. Yakovenko O, et al. Proc Natl Acad Sci USA. 2015 Aug. 11; 112(32):9884-9.
33. Wellens A, et al. PloS One. 2008 Apr. 30; 3(4):e2040.
34. Bucher D, et al. PLoS Comput Biol. 2011 April; 7(4):e1002034.
35. Standfuss J, et al. Nature. 2011 Mar. 31; 471(7340):656-60.
36. Xiong J P, et al. J Biol Chem. 2000 Dec. 8; 275(49):38762-7.
37. Vanatta D K, et al. Nat Commun. 2015 Jun. 15; 6:7283.
38. dela Cruz R G C, et al. J Biol Chem. 2006 Oct. 20; 281(42):31668-76.
39. Kim J, et al. Sci Adv. 2017 April; 3(4):e1600663.
40. Dellisanti C D, et al. J Biol Chem. 2011 Feb. 4; 286(5):3658-70.
41. Brüschweiler S, et al. ACS Chem Biol. 2013 Jul. 19; 8(7):1600-10.
42. Mairey E, et al. J Exp Med. 2006 Aug. 7; 203(8):1939-50.
43. Pappelbaum K I, et al. Circulation. 2013 Jul. 2; 128(1):50-9.
44. Smyth C J, et al. FEMS Immunol Med Microbiol. 1996 Dec. 1; 16(2):127-39.
45. Busch A, et al. Philos Trans R Soc Lond B Biol Sci. 2012; 367(1592):1112-22.
46. Sokurenko E V, et al. J Biol Chem. 1997 Jul. 11; 272(28):17880-6.
47. Kisiela D I, et al. PLoS Pathog. 2015 May; 11(5):e1004857.
48. Delaglio F, et al. J Biomol NMR. 1995 November; 6(3):277-93.
49. Johnson B A, Blevins R A. J Biomol NMR. 1994 September; 4(5):603-14.
50. Vanwetswinkel S, et al. J Med Chem. 2014 Feb. 27; 57(4):1416-27.
51. Krissinel E, Henrick K. Acta Crystallogr D Biol Crystallogr. 2004 December; 60:2256-68.
52. Winn M D, et al. Acta Crystallogr D Biol Crystallogr. 2011 April; 67(Pt 4):235-42.
53. Brooks B R, et al. J Comput Chem. 2009 Jul. 30; 30(10):1545-614.
54. Gaines J C, et al. Protein Eng Des Sal PEDS. 2017 May 1; 30(5):387-94.
55. Ribeiro J, et al. Bioinforma Oxf Engl. 2015 Aug. 15; 31(16):2751-3.
56. Bouckaert J. et al. Mol Microbiol. 2005 January; 55(2):441-55.
57. Kalé L, et al. J Comput Phys. 1999 May 1; 151(1):283-312.
58. MacKerell A D, et al. J Phys Chem B. 1998 Apr. 30; 102(18):3586416.
59. Schneider T, Stoll E. Phys Rev B. 1978 Feb. 1; 17(3):1302-22.
60. Feller W. An introduction to probability theory and its applications, vol 2. John Wiley & Sons; 2008.
61. Schlitter J, et al. Mol Simul. 1993 Aug. 1; 10(2-8):291-308.

### Example 2: Additional Charged Amino Acids can be Substituted for the Hydrophobic Amino Acid to Lock the Conformation of an Allosteric Protein

The use of lysine in the place of glutamic acid as the replacement for position 34 in FimH was tested as demonstrated in Example 1. Lysine exhibited a better conformation-locking effect, but this substitution operated by much the same principle.

Mannose induces conformational shift of the L34K mutant towards the active state.

The observation that the L34K mutation de-activates the active state-FimH^{FocH} hybrid could be because the mutated lectin domain adopts an inactive state conformation or the mutation could cause domain mis-folding and, therefore, loss of function. We assessed the effect of the L34K mutation on the conformation of purified pilin domain-free lectin domain (residues 1-160 with N-terminal His-tag) by NMR. Peaks in the ¹⁵N-HSQC spectrum of the L34K lectin domain are well dispersed, indicating that the protein exists in a distinct and well-folded conformation. Notably, the L34K mutant spectrum differs from that of the wild-type lectin domain at virtually every resonance, indicating that the L34K mutant assumes a different conformation from the wild-type, which is in the active state in the absence of pilin domain.

Addition of soluble mannose does not induce extensive chemical shift changes in the spectrum of wild-type FimH lectin domain, in accordance with previous studies. Residues that are perturbed are localized in the mannose binding pocket only. In stark contrast, addition of mannose to L34K lectin domain results in extensive chemical shift perturbations throughout the spectrum, suggestive of a global ligand-induced conformational change. Moreover, the spectra of wild-type and L34K lectin domains in the presence of mannose overlap across >90% of residues, with only ten residues (mostly around the mutated residue) having significant chemical shift perturbation. Thus, the NMR results indicate that mannose binding induces a global shift in the L34K lectin domain towards an active conformation, strongly supporting the hypothesis that the substitution of L34 with a charged lysine stabilizes an inactive-like (“L34-out”) conformation of the lectin domain.

### Example 3: ELISA Assays

To test the conformation-specific antibody (mAb21) binding to FimH, microtiter plate wells were coated with purified fimbriae at a concentration of 0.1 mg/ml in 0.02 M NaHCO3 buffer for 1 h at 37° C. The wells were washed with PBS and quenched for 20 min with 0.2% (wt/vol) BSA in PBS. The immobilized fimbriae were incubated with serial dilutions of pure mAbs for 1 h, and after washing, bound antibodies were detected with a 1:3,000 diluted HRP-conjugated goat anti-mouse antibody (Bio-Rad). The reaction was developed at room temperature using 3,3′,5,5′-tetramethylbenzidine (TMB, SeraCare's KPL, and absorbance was read at 650 nm. In some experiments, binding of the antibodies was examined in the presence of 1% of αmm. All incubation steps in ELISA assays were performed at 37° C. unless stated otherwise.

To test the effect of mannose ligand (α-methyl-D-mannopyranoside, αmm) on conformational state of fimbrial FimH, mAb21 at a concentration of 0.5 μg/ml was added to fimbriae-coated wells in the absence or presence of serial dilutions of αmm. Binding of the latter antibody was detected as above.

### Example 4: Bacterial Adhesion

FmIH-dependent bacterial adhesion was analyzed as described previously with minor modifications [Kisiela D I, et al. (2015) Inhibition and Reversal of Microbial Attachment by an Antibody with Parasteric Activity against the FimH Adhesin of Uropathogenic E. coli. PLoS Pathog 11: e1004857]. Briefly, Immulon 4HBX microtiter plates (ThermoFisher Scientific) were coated with 20 μg/mL of asialofetuin (Sigma-Aldrich) and plates were quenched with 0.2% BSA in PBS. Bacterial suspensions in PBS (OD540=2) were added to each well in a volume of 100 μl and plates were spun for 2 min at 750×g. After extensive washing, bound bacteria were stained with 0.1% (vol/vol) crystal violet for 20 min at room temperature and were washed several times with water. Then 100 μl of 50% (vol/vol) ethanol was added to each well, and the absorbance at a wavelength of 600 nm was measured. For inhibition, bacterial binding was tested in the presence of 1% galactose (Sigma).

### Example 5: Horseradish Peroxidase (HRP)-Binding Assay

To test functional activity of FimH variants carrying different amino acid substitutions, 96-well microtiter plates were coated overnight with purified type 1 fimbriae in 0.02 M NaHCO3 buffer at pH 9.6 (0.1 mg/ml). The immobilized fimbriae, after blocking with 0.2% BSA in PBS, were incubated with horseradish peroxidase (HRP) at a concentration of 50 μg/ml for 1 h in the absence or presence of 1% αmm. After extensive washing with PBS, bound HRP was detected by adding 100 μl of TMB substrate and absorbance was read at 650 nm.

### Example 6: Biofilm Formation Assay

Microtiter 96-well plates were coated with 20 μg/mL of yeast mannan or RNaseB (Sigma-Aldrich) in 0.02 M NaHCO₃ buffer at pH 9.6. Bacterial strains grown overnight in 3 ml LB media were spun and washed 1× with minimal essential media (MEM, Difco). Bacterial suspensions in MEM, at final concentration OD=0.2, were added to mannan-coated wells in the absence and presence of 52 mM mannose or 50 μg/ml mAbs and incubated 16 h at RT without shaking. After washing with PBS, formed biofilms were stained with 0.1% (v/v) crystal violet (Becton Dickinson) as described above for bacterial adhesion assay. For biofilm detachment, 16 h-old biofilm produced by E. coli UTI89 on mannan-coated microtiter plates was washed 3 times with PBS and incubated in the absence or presence of 1% mannose or 50 μg/ml mAbs at RT with mild shaking. The wells were washed 3 times with PBS and stain for biofilm detection as described above.

### Example 7: Allosteric Mechanism of a Fast and Strong Bacterial Adhesion

Attachment of microbial pathogens to host cells and tissues requires that their adhesion proteins bind both quickly and strongly to target cell receptors. FimH protein of Escherichia coli is a model two-domain adhesin where the mannose-binding lectin domain switches allosterically from a ‘low-affinity state’ conformation with a fast on-rate for binding mannose to a ‘high-affinity state’ conformation with a slow off-rate for detachment from mannose. Under fluidic shear stress, this conformational switch creates an allosteric catch bond mechanism of adhesion because tensile force favors separation of the lectin domain from the anchoring pilin domain that is an allosteric effector of the former. In this Example, we used a recombinant FimH variant with a weakened inter-domain interaction and show that a fast and sustained allosteric activation of FimH can occur not only under shear but also under static conditions, without the application of tensile force. Moreover, it appears that the conformational shift from the low- to high-affinity state is an intrinsic property of the lectin domain independent from its ability to interact with the pilin domain or the presence of mannose. Thus, the allosteric catch bond mechanism is not limited to a shear-dependent, force-induced mode of adhesion but can be a much broader phenomenon involved in cell-pathogen attachments under a broad range of hydrodynamic conditions. This concept that allostery can enable more effective receptor-ligand interactions in various physiological environments is fundamentally different from the conventional wisdom that allostery provides a mechanism to turn binding off under specific conditions.

Attachment of bacterial or viral pathogens to host cells is critical for tissue colonization and intracellular invasion by pathogens, formation of biofilms, etc [1]. The adhesion generally involves the attachment proteins (also called “adhesins”) and their counterpart ligands on the surface of target cells (also called “receptors”), typically of a protein or glycan nature. The initiation of cell adhesion generally occurs under hydrodynamic conditions, which require that adhesive bonds be established quickly and, at the same time, be strong enough to resist pathogen detachment under flow [2, 3]. Based on structural data, many microbial and eukaryotic adhesive proteins undergo long-range conformational changes associated with the receptor binding [4, 5, 6]. These conformational changes are reminiscent of allosteric proteins, but the functional significance of allostery in initiating and sustaining cell attachment under different physiological conditions is still poorly understood.

The FimH protein is the most prevalent adhesin of Escherichia coli positioned on the tips of surface-exposed hair-like organelles called fimbriae (or pili) and can bind mannosylated oligosaccharides of cell surface glycoproteins [7]. FimH has a structure typical for fimbrial adhesins, with two β-sandwich-shaped domains—a distal N-terminal lectin domain that contains the mannose-binding pocket and a C-terminal pilin domain that anchors the adhesin to the fimbrial tip [8]. FimH is a model allosteric adhesin with the lectin domain able to assume two end-point conformational states [8, 9, 10]. In the ‘inactive’ conformer or ‘low-affinity state’ (LAS), the lectin domain closely interacts with the pilin domain and has a slightly compressed structure with the mannose-binding pocket being wide open on the top of domain. In contrast, in the ‘active’ conformer or ‘high-affinity state’ (HAS) the domains do not interact, and the lectin domain adopts a more elongated β-sandwich conformation with a narrowed mannose-binding pocket. The pilin domain acts as the allosteric effector of the lectin domains that stabilizes it in LAS, so that separation of the two domains is required for the switch from LAS to HAS, i.e. the FimH activation [9, 11]. So far, all structures of FimH in the LAS were obtained in the absence of bound ligand, while structures of HAS have been obtained with or without mannose [10, 12]. A hybrid conformer of FimH has also been described with a bound ligand, where the lectin domain has an intermediate conformation with the closed pocket but a fully preserved interdomain interaction [10, 13]. The wide configuration of mannose-binding pocket in LAS mediates a fast on-rate but also a fast off-rate for ligand binding [2], while HAS mediates the binding with a much slower on- and off-rate. Thus, the conversion from LAS to HAS by tensile force is the basis for both fast and strong bacterial adhesion under flow [2].

It has been shown that while the LAS conformation of the lectin domain dominates in full length FimH without mannose, addition of soluble mannose leads to an increased prevalence of HAS due to the allosteric switch from LAS [14, 15]. However, it was also demonstrated that the FimH mediated bacterial adhesion, especially via terminal mono-mannose (Man1) moieties of oligosaccharides, depends on shear, i.e., it is fairly weak under static or low shear conditions but becomes considerably stronger as shear increases [16, 17]. Furthermore, single molecule force spectroscopy studies indicated that the FimH-mannose interaction is significantly enhanced if mechanical force is applied between the bound mannose and fimbriae [2, 18, 19]. Thus, it has been postulated that application of mechanical tensile force is necessary to induce separation of the lectin and pilin domains and, thus, for the FimH activation to happen [2, 10, 20, 21]. Because the FimH-mannose interaction is force-enhanced, it was defined that FimH forms so-called ‘catch bonds’ to distinguish them from slip-bonds which become weaker under tensile force [21, 22, 23, 24, 25]. After the catch bond mode of binding was shown for FimH [16], it was demonstrated or implied for a great variety of adhesive or structural interactions, including integrins [26, 27, 28], cadherins [29, 30], selectins [31, 32, 33], T-cell receptors [34, 35], cytoskeletal proteins [36, 37, 38] or matrix proteins [39], and many bacterial adhesins in different species [9, 25, 40, 41, 42, 43, 44, 45, 46, 47, 48). Based on the FimH studies, it was postulated that, at least in certain cases, the catch bond mechanism could have an allosteric basis, where the central role is played by a conformational switch of the adhesive domain (like the lectin domain of FimH) upon separation from the effector domain (like the pilin domain). However, FimH remains the only bacterial adhesin with structurally resolved allosteric conformers. Thus, it remains unclear whether the FimH-like allosteric mechanism of catch bond is broadly occurring in nature, to what extent the shear stress is essential for the catch bond manifestation and what is the exact role of allosteric interplay between the ligand binding, domains separation and tensile force in the fast formation of strong adhesive bonds.

It is important to understand whether the allosteric catch bond-like mechanism of the adhesin activation is only manifested in a shear-dependent binding phenotype or, alternatively, could be involved in cell adhesion under no or low flow conditions as well. While the shear-dependent adhesion mode could be advantageous for fast surface spreading and biofilm formation of bacteria [48, 50, 51], it also limits the ability of bacteria to attach in host compartments where shear is low. For example. FimH of E. coli isolated from patients with urinary tract infections (UTIs) often have amino acid changes that enable a more effective adhesion under static conditions and increase the bacterial virulence in mouse model of UTI [12, 52, 53]. This could be due to the fact that urine flow inside the bladder lumen, ureters or renal pelvis is too slow to provide a sufficient shear for the shear-dependent mode of bacterial attachment to urothelium. However, engineered variants with even stronger static adhesion displayed decreased virulence in a mouse model of UTI [12]. It remains unknown whether or how the FimH allosteric properties remain important in mediating enhanced adhesive interactions under no shear.

This Example analyzes in detail the allosteric kinetics and mannose-specific binding mediated by four structural variants of FimH—two of a native structure and two with a recombinant structure of the pilin domain with a less stable interaction with the lectin domain. We show that with a preserved but eased interdomain interaction, FimH binds rapidly and yet strongly to mannose even in the absence of tensile force, allowing bacteria to readily and stably adhere in a wide range of hydrodynamic conditions. This rapid and strong binding requires the allosteric shift from LAS to HAS once mannose has bound. This shift appears to occur without tensile force at a rate independent of mannose binding or the intrinsic strength of the inter-domain interaction. At the same time, the stability of the HAS state is greatly enhanced by both mannose-binding and interdomain interactions. Thus, these data suggest that the ability to form allosteric catch bonds is a much broader phenomenon among adhesive proteins than originally thought.

### Materials and Methods

Bacterial Strains, Fimbriae and Reagents

The recombinant Escherichia coli strain K12 (AAEC191A), with deleted fim cluster, carrying pPKL114 plasmid containing the entire fim gene cluster from the E. coli strain K12 (but with the inactivated fimH gene) and pGB2-24-based plasmid with naturally occurring fimH alleles from E. coli F18 and E. coli K12 were described previously [20, 59, 67]. Creation of the 16 amino acid FocH insertion into the pilin domain of naturally occurring fimH alleles were described previously [20, 59]. For type 1 fimbriae expression, bacteria were grown overnight in LB cultures with mild shaking. The type 1 fimbriae were isolated from spun bacterial cultures using a magnesium chloride precipitation method [68]. Yeast mannan, alpha-methyl-mannoside (mannose for further) and horseradish peroxidase (HRP) were purchased from Sigma-Adrich. Monoclonal antibodies mAb824 and mAb21 were expressed and purified from serum-free hybridoma cell cultures as described elsewhere [61, 68]. Briefly, antibodies from culture supernatants were first collected using protein G-agarose (Millipore) followed by size-exclusion chromatography using Superdex 200 (GE Healthcare Life Sciences).

Bacterial Adhesion Assay

FimH-dependent bacterial adhesion was analyzed as described previously [69]. Briefly, Immulon 4HBX microtiter plates (Thermo Electron Corp.) were coated with 20 μg/mL of yeast mannan and plates were quenched with 0.2% BSA in PBS. Bacterial suspensions in PBS (OD₅₄₀=2) were added to each well in a volume of 100 μl and incubated for 1 h at 37° C. After extensive washing, bound bacteria were stained with 0.1% (vol/vol) crystal violet for 20 min at room temperature and were washed several times with water. Then 100 μl of 50% (vol/vol) ethanol was added to each well, and the absorbance at a wavelength of 600 nm was measured. For inhibition, bacterial binding was tested in the presence of 1% mannose.

HRP and Antibody Binding to Immobilized Fimbriae

The assays were performed as described previously. In brief. 96-well microtiter plates were coated overnight with purified type 1 fimbriae in 0.02 M NaHCO₃ buffer at pH 9.6 (0.1 mg/ml), followed by washes and quenching with 0.2% BSA in PBS. The immobilized fimbriae were incubated with HRP at a concentration of 50 μg/ml for 1 h in the absence or presence of 1% mannose. After extensive washing with PBS, bound horseradish peroxidase was detected by adding 100 μl of TMB substrate (Kirkegaard and Perry Laboratories, KPL) and absorbance was read at 650 nm. In case of mAb21, the antibody solution was added at a final concentration of 1 μg/mL after the fimbriae were combined and incubated for 5 min with 50 μl PBS without or with 1% mannose. After the incubation for indicated time, the plates were washed extensively with PBS and FimH-mAb21 complexes were detected using HRP-conjugated goat anti-mouse antibody (Sigma-Aldrich, diluted 1:3000). All incubations, unless stated otherwise, were performed at 37 C. The colorimetric reaction was developed in the presence of TMB substrate at RT and absorbance was read at 650 nm. To test the effect of mAb824 on HRP binding to fimbrial FimH, immobilized fimbriae were pre-incubated for 45 min with the antibody that were washed out before the HRP addition.

Red Blood Cell (RBC) Aggregation Assay

Guinea pig RBC (Colorado Serum Company) were washed with PBS three times. 50 μl of RBC suspension (1%) was mixed 1:1 with 50 μl of 4×10⁸ cfu of type 1 fimbriated bacteria in a glass plate in the absence and presence of 1% αmm and continuously mixed by gently rocking at ˜2-3 s⁻¹. The aggregation of RBC was visually scored and the time at which noticeable aggregation was observed and reported in minutes (min). In some experiments, bacterial cells were first pre-incubated for 5 min with 67 μg/ml of mAbs in the glass plate and then mixed with RBC suspension in the absence and presence of 1% αmm. RBC rosette-formation assay under was performed by mixing equal amounts of serially diluted bacterial sus-pensions (starting from OD540 nm 1.0) and a 1% suspension of RBC in U-bottom microtiter plate wells as described previously.

X-Ray Crystallography

LD^{wt}-PD^{FocH} fimbrial tips (FimH-FimG-FimF-FimF-FimC) were purified as described previously for LD^{wt}-PD^{wt} fimbrial tips and crystallized under the same conditions by using hanging drop vapor diffusion techniques at room temperature. Crystals were obtained by mixing equal volume drops containing the protein (11 mg/ml in 20 mM Hepes pH 7.5) and a crystallization solution containing 1.8M KCl, 0.1M sodium citrate pH 4.2. Crystals were transferred to a cryosolution containing 30% glycerol before freezing at 100 K in a nitrogen stream for diffraction data collection.

Diffraction data were collected at SSRL B:9-2 (λ=0.98 Å) in June, 2009. The space group for the crystals is R32 with two copies of the tip complex in the asymmetric unit. The diffraction data were processed with HKL2000 [70]. Data set statistics are shown in the Table 9.

TABLE 9
X-ray diffraction data set statistics

| beamline | SSRL 9-2 (0.98 Å wavelength) | |
|---|---|---|
| # Reflections measured | 2948484 | |
| # Unique Reflections | 109868 | (9804) |
| Resolution Range | 50.0-2.8 Å | (2.90-2.80) |
| Overall Completeness | 94.4% | |
| Completeness, last shell | 85.0% | (2.9-2.8 Å) |

| | |
|---|---|
| Redundancy | 4.2 |
| Redundancy, last shell | 3.0 |
| I/σ(I), overall | 9.38 |
| I/σ(I), last shell | 1.28 |
| R_{merge}, overall | 0.139 |
| R_{merge}, last shell | 0.769 |

The initial structural model was derived from the isomorphous FimH tip structure (PDB 3JWN) with adjustments for the different sequence from residues 186 to 201 (derived from the FocH adhesin). The model for this region with the correct sequence was obtained during refinement using REFMAC-5 [71] in the CCP4 suite [72]. Rfree [73] was calculated using 5% of the data in the test set. At 2.9 Å resolution, the data set completeness drops below 90% and I/σ(I) is 1.8. Significant diffraction data were obtained out to 2.8 Å resolution and we chose to use all reflections to that limit for refinement. Sigma A weighted |Fo|−|Fc| and 2|Fo|−|Fc| electron density maps [74] were viewed with XtalView [75] and Coot [76] for graphical evaluation of the model and electron density maps. Table 10 contains refinement statistics for the structure.

TABLE 10
X-ray diffraction data refinement statistics

| Resolution | 50.0-2.80 | Å |
|---|---|---|
| Number of reflections, working set | 98867 | |
| Number of reflections, test set | 5492 | |
| R_{cryst}, all data | 0.246 | |
| R_{cryst}, working set | 0.245 | |
| R_{free}, all data | 0.274 | |
| Bond RMS (restrained) | 0.006 | Å |

| | |
|---|---|
| Number of protein atoms | 13859 |
| Number of solvent atoms | 35 |

Accession Numbers

PDB: 7SZO

Molecular Dynamics Simulations

Two 50-ns long simulations were performed with LD^{wt}-PD^{wt} and two also 50-ns long runs were performed with LD^{wt}-PD^{FocH}. The initial conformations were derived from PDB code 3JWN for LD^{wt}-PD^{wt} and PDB code 7SZO for LD^{wt}-PD^{FocH}, respectively. For both structures, chain H was used (which contains LD and the portion of PD that is covalently bound to LD) and the 13 N-terminal residues of chain G (which contains a strand donated to PD from the FimG subunit). The MD simulations were performed with the program NAMD [77] using the CHARMM22 force field [78, 79, 80] and the TIP3P model of water. Each protein was solvated using a cubic water box with side length of 115 Å and periodic boundary conditions. Chloride and sodium ions were added to neutralize the system and approximate a salt concentration of 150 mM. The resulting systems contained each in total ca. 146,000 atoms. After solvation, each system underwent 500 steps of minimization while the coordinates of the heavy atoms of the protein were held fixed and subsequent 500 steps with no restraints. Each simulation was started with different initial random velocities to ensure that different trajectories were sampled whenever starting with the same initial state. Electrostatic interactions were calculated within a cutoff of 10 Å, while long-range electrostatic effects were taken into account by the Particle Mesh Ewald summation method [81]. Van der Waals interactions were treated with the use of a switch function starting at 8 Å and turning off at 10 Å. The dynamics were integrated with a time step of 2 fs. The covalent bonds involving hydrogens were rigidly constrained by means of the SHAKE algorithm with a tolerance of 10⁻⁸. Snapshots were saved every 10 ps for trajectory analysis. Before production runs, harmonic constraints were applied to the positions of all heavy atoms of the protein to equilibrate the system at 300 K during a time length of 0.2 ns. After this equilibration phase, the harmonic constraints were released. After this equilibration phase, the harmonic constraints were released, and simulation runs were performed for a total of 50 ns each. During both the equilibration and production phases, the temperature was kept constant at 300 K by using the Langevin thermostat [82] with a damping coefficient of 1 ps⁻¹, while the pressure was held constant at 1 atm by applying a pressure piston [83].

Quantitative Model of mAb21 Binding to FimH.

For our modeling, we made the following definitions and assumptions. LAS′ represents the fraction of immobilized FimH that is in any conformation that is not recognized by mAb21, whether or not in the presence of mannose, such as the LAS or mannose-bound hybrid conformation. HAS represents the fraction of immobilized FimH in a conformation that can be recognized by mAb21, such as unbound or mannose-bound HAS. Bound represent the fraction of immobilized FimH bound to mAb21. The solution concentration of mAb21 was assumed to be constant, because the concentration added to the assays (6.7*10⁻⁹ M), is a large excess over immobilized FimH so would not be depleted by the binding reaction. Finally, the unbinding reaction was not included in the model because mAb21 did not significantly detach from HAS in the time course of these assays [61].

The rate constant for HAS binding to mAb21 in the presence of 6.7*10⁻⁹ M of mAb21 is k_{mAb21}, the rate constant for activation from LAS to HAS is k_{act}, and the rate constant for deactivation from HAS to LAS is k_{deact}. It is expected that k_{mAb21} is the same for all variants in the absence or presence of mannose, because these structural differences do not directly impact the mAb21 epitope [61], and mAb21 was always added at the same concentration. To estimate k_{mAb21}, we assumed that LD^{A27V}-PD^{FocH} is essentially all in HAS in the presence of mannose, so we fit high time-resolution data with a saturable binding model,

$$\frac{dBound}{dt} = {k}_{mAb 21} * ( 1 - Bound ) ,$$

with Bound(0)=0. This resulted in a value k_{mAb21}=0.04 sec⁻¹.

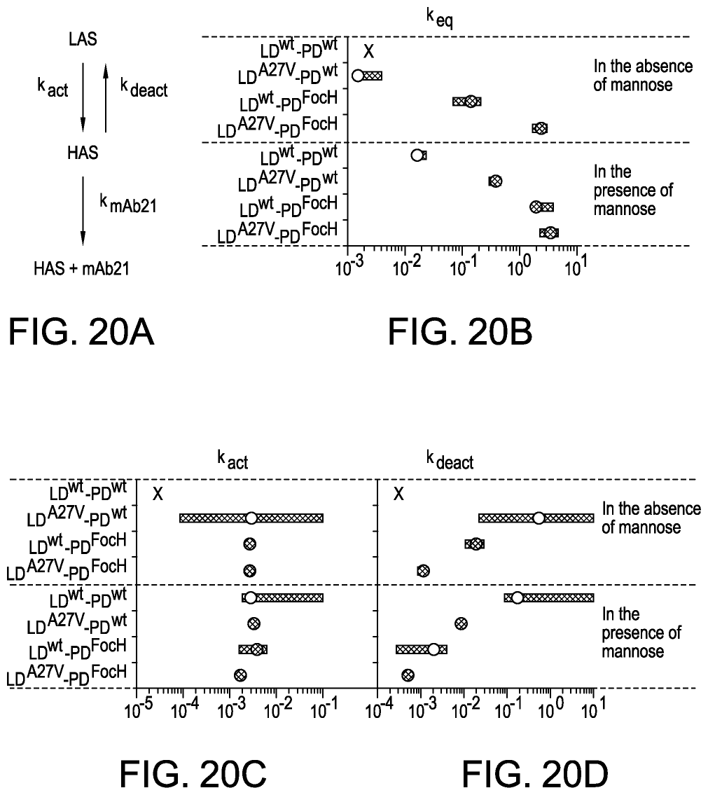

These assumptions resulted in the following model, which is also illustrated in FIG. 20A, and was implemented using a custom MATLAB script.

$$\begin{matrix}\frac{dLAS}{dt} = - {k}_{act} * LAS + {k}_{deact} * HAS & Eq . 1 \\ \frac{dHAS}{dt} = {k}_{act} * LAS - {k}_{deact} * HAS - {k}_{mAb 21} * HAS & Eq . 2 \\ \frac{dBound}{dt} = {k}_{mAb 21} * HAS & Eq . 3\end{matrix}$$

Because FimH was pre-incubated with mannose, while mAb21 was added at time 0 to start the assay, the initial conditions of the model reflect the assumption that LAS and HAS are at equilibrium and there is no FimH bound to mAb21:

$$\begin{matrix}LAS ( 0 ) = \frac{{k}_{deact}}{{k}_{deact} + {k}_{act}} & Eq . 4 \\ HAS ( 0 ) = \frac{{k}_{act}}{{k}_{deact} + {k}_{act}} & Eq . 5 \\ Bound ( 0 ) = 0 & Eq . 6\end{matrix}$$

We then estimated k_{act} and k_{deact} for all four variants in the presence and absence of mannose by fitting this model to the data by minimizing the least square error between the model and data points divided by the value of the data. Parameters were estimated independently for the data from each day the experiment was performed, to provide multiple values of each constant. The mean and standard error of the mean of these values is shown in Table 8.

TABLE 8
Parameters estimated from the model.

| variant | mannose | n | k_{act} ± SEM | k_{deact} ± SEM | k_{eq} ± SEM |
|---|---|---|---|---|---|
| LD^{wt}-PD^{wt} | absence | 0 | N.D. | N.D. | N.D. |
| LD^{A27V}-PD^{wt} | absence | 3 | >8.8 × 10⁻⁵ | >2.3 × 10⁰ | 1.5 × 10⁻³ to 4.0 × 10⁻³ |
| LD^{wt}-PD^{FocH} | absence | 4 | 2.9 × 10⁻³ ± 0.5 × 10⁻³ | 2.0 × 10⁻² ± 0.9 × 10⁻² | 1.4 × 10⁻¹ ± 0.7 × 10⁻¹ |
| LD^{A27V}-PD^{FocH} | absence | 3 | 2.8 × 10⁻³ ± 0.3 × 10⁻³ | 1.2 × 10⁻³ ± 0.3 × 10⁻³ | 2.4 × 10⁰ ± 0.7 × 10⁰ |
| LD^{wt}-PD^{wt} | presence | 1 | >2.0 × 10⁻⁴ | >8.5 × 10⁻² | 1.7 × 10⁻² to 2.3 × 10⁻² |
| LD^{A27V}-PD^{wt} | presence | 3 | 3.4 × 10⁻³ ± 0.4 × 10⁻³ | 8.7 × 10⁻³ ± 1.7 × 10⁻² | 3.9 × 10⁻¹ ± 0.9 × 10⁻¹ |
| LD^{wt}-PD^{FocH} | presence | 4 | 4.1 × 10⁻³ ± 0.2 × 10⁻³ | 2.1 × 10⁻³ ± 1.8 × 10⁻³ | 2.0 × 10⁰ ± 2.0 × 10⁰ |
| LD^{A27V}-PD^{FocH} | presence | 3 | 1.8 × 10⁻³ ± 0.2 × 10⁻³ | 5.1 × 10⁻⁴ ± 1.0 × 10⁻⁴ | 3.6 × 10⁰ ± 1.2 × 10⁰ |

There were two limitations to using this model to estimate these parameters. First, mAb21 did not measurably bind to LD^{wt}-PD^{wt} in the absence of mannose, so no parameters could be obtained for this condition. Second, in two additional conditions—LD^{wt}-PD^{wt} in the presence of mannose and LD^{A27V}-PD^{wt} in the absence of mannose, a wide range of values of the rate constants produced the same fitting error. To identify a lower bound for the rate constants, we fit k_{act} while holding k_{deact} constant over a wide range of values and observed that the error was high for low rates but reached a plateau at higher rates. We identified the lowest k_{deact} and corresponding k_{act} values at which the error was no more than 10% that of all higher values. Throughout this plateau, K_{eq}=k_{act}/k_{deact} remained within a tight and reproducible range, so was well-determined by the model. For these two conditions, the lower bounds for the two kinetic constants and the upper and lower bound for the equilibrium constants are reported in Table 8.

For the remaining five conditions, the rate constants as well as equilibrium constants were reproducible for a wide range of initial guesses, and between replicate experiments, so we consider these to be reliable. We noticed that k_{act} was similar for all five variants for which it could be measured, and over tenfold slower than k_{mAb21}, demonstrating that this rate constant is not simply a reflection of the rate of antibody binding, but rather reflects intrinsic conformational changes in the protein. For the two conditions that did not result in rate constants, we found it useful to estimate the value of k_{deact} and K_{eq} assuming that k_{act} was equal to the average value of the other five conditions. These values were consistent with the estimated bounds and are also reported in FIGS. 20C and 20D for illustration.

Knowledge Used to Sketch the Energy Landscapes.

The curves reflect the following previously established knowledge. The HAS well is lower in the blue than the black curves due to more favorable interactions between mannose and the pocket in HAS relative to LAS [9]. The HAS energy well is even lower in the orange curves, because tensile force is expected to decrease the relative energy of the longer state by the product of the force and the difference in lengths between the states [84], and HAS is longer than LAS [9]. Finally, tensile force has been shown to enhance the rate that single FimH bonds convert to HAS [18, 21], so force must decrease the energy of the unknown transition state between LAS and HAS. Moreover, the differences between the curves for each variant are the same because none of the point mutations that distinguish these variants could impact either the interactions with mannose nor the lengths for of any of the states. The curves also reflect the following new knowledge from this manuscript. The transition state energy is the same in the LAS and HAS state for all blue and black curves because the rate k_{act} was the same for all variants and with and without mannose. The relative height of the HAS and LAS wells with and without mannose approximately reflect the equilibrium constants in the absence of mannose.

### Results

Structural Alteration of Pilin Domain Results in Strong Binding without Shear.

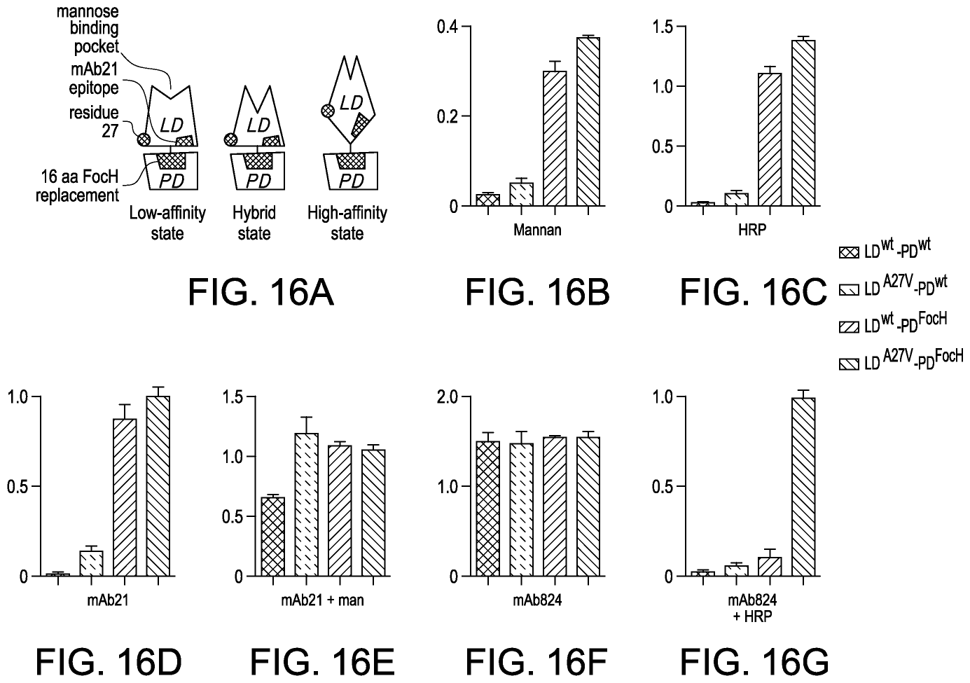

As described previously [9, 10, 12, 54], FimH adhesin can assume at least three conformational states—the end-point LAS and HAS states, with low- and high-affinity binding to mannose, respectively, and an intermediate hybrid state (FIG. 16A). In this study, we focused on four structural variants of FimH adhesin differing in the structure of lectin domain (LD) and/or pilin domain (PD). LD^{wt}-PD^{wt} is a naturally-occurring FimH derived from model fecal E. coli strain F18 that represents the evolutionary ‘wild-type’ and, overall, the most common structural FimH variant among E. coli [52, 53, 55, 56, 57] (FIG. 16A). LD^{A27V}-PD^{wt} is also a naturally occurring variant that is often found among uropathogenic strains and expressed by the model E. coli strains K12 and J96 [55]. In LD^{A27}-PD^{wt}, there is a valine residue instead of alanine at position 27 located at the edge of interdomain region (FIG. 16A). The LD^{wt}-PD^{FocH} and LD^{A27V}-PD^{FocH} variants have the pilin domain of recombinant nature (PD^{FocH}), where the lectin domains are fused to a structurally altered pilin domain, in which 16 residues (186-YCAKSQNLGYYLSGTT-201; SEQ ID NO: 33) in PD^{wt} were replaced with a homologous (38% identity) sequence (RCDQTQSVSYTLSGSV; SEQ ID NO: 34) from the FocH adhesin, which is a distant structural homologue of FimH [20, 58]. The replacement in PD^{FocH} does not affect the FimH incorporation into the fimbriae [20, 58].

Under static, no shear conditions, bacteria expressing both naturally-occurring FimH variants adhere only weakly to Mani-rich model glycoprotein—yeast mannan (FIG. 16B). In contrast, both recombinant variants with the altered pilin domain mediated a strong adhesion to mannan. Analogously to the bacterial adhesion results, soluble horseradish peroxidase (HRP, another Mani-rich model glycoprotein) demonstrated only marginal binding to a fimbrial carpet with the naturally occurring FimH variants but bound much more strongly to fimbriae with the recombinant FimH variants (FIG. 16C).

We next used monoclonal antibody mAb21 that recognizes the HAS conformation of FimH with its binding epitope located in the interdomain region of the lectin domain (FIG. 16A) but does not recognize LAS [59]. mAb21 did not bind fimbriae with naturally occurring FimH but recognized well both LD^{wt}-PD^{FocH} and LD^{A24V}-PD^{FocH} (FIG. 16D). At the same time, binding of mAb21 to the naturally occurring variants became pronounced if the binding was done in the presence of 1% alpha-methyl-mannoside (hereafter called mannose) (FIG. 16E). Notably, however, in the presence of soluble mannose, the binding of mAb21 to LD^{wt}-PD^{wt} was somewhat lower than that to LD^{A27V}-PD^{wt} and the recombinant variants.

Taken together, the results of the mannan adhesion, HRP-binding and mAb21-recognition assays suggest that in the absence of soluble mannose, both lectin domain variants adopt the LAS in the background of PD^{wt}. At the same time both lectin domains in the background of PD^{FocH} demonstrate a HAS-like phenotype. These results are consistent with previous characterization of the FimH variants indicating that, in native FimH and in the absence of shear force, the PD^{wt} interaction with the lectin domain allosterically inhibits the switch from LAS to HAS, while the effector function of PD^{FocH} is significantly reduced [20]. Also, in the PD^{wt} background, LD^{A27V} appears to be more activatable by mannose relatively to LD^{wt}, though no difference was obvious between the LDs in PD^{FocH} background in this assay.

LD^{wt}-PD^{FocH} is in the LAS Conformation in the Absence of Mannose.

We have recently shown that, unlike the HAS-specific mAb21, mAb824 recognizes equally well both lectin domain conformations but stabilizes the conformation that it is bound to, functioning as an allosteric kinetic trap [60]. Thus, we probed the conformational state of the FimH variants by treating the surface-immobilized fimbriae with mAb824 and then testing their ability to bind soluble HRP. As expected, mAb824 recognized equally well all fimbriae-incorporated FimH variants (FIG. 16F). In complex with mAb824, binding of HRP to LD^{wt}-PD^{wt} and LD^{A27V}-PD^{wt} remained very weak and to LD^{A27V}-PD^{FocH} relatively strong (FIG. 16G), indicating that in the absence of mannose, the former two are predominately in LAS, while the latter is mostly in the active state. Surprisingly, mAb824-pretreated LD^{wt}-PD^{FocH} had a drastically reduced ability to bind soluble HRP relatively to the untreated LD-PD^{FocH} (FIG. 16G, compared to 1C). This suggests that, despite displaying a mannose-binding phenotype like that of the LD^{A27V}-PD^{FocH}, the conformation of LD^{wt}-PD^{FocH} is predominantly LAS in the absence of mannose, similar to the conformation of naturally occurring FimH.

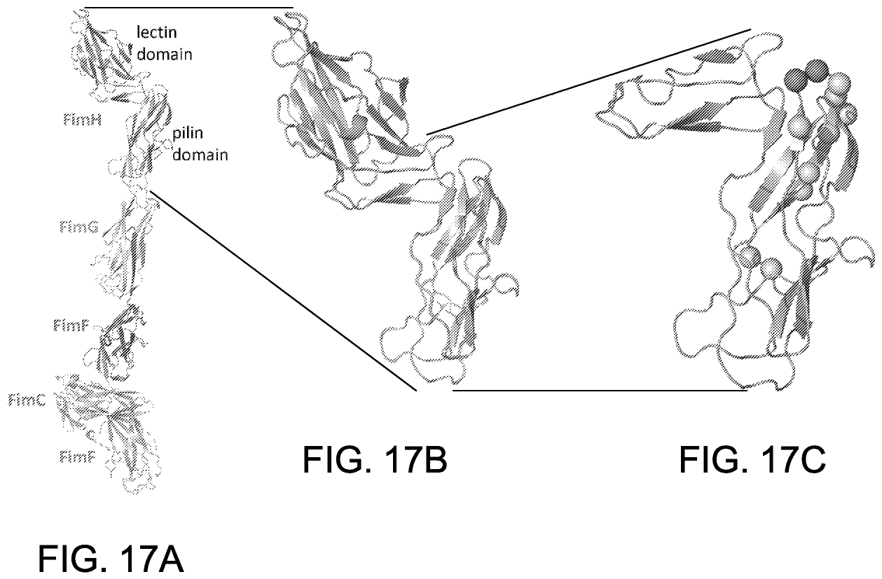

We attempted to obtain the crystal structures of LD^{wt}-PD^{FocH} and LD^{A27V}-PD^{FocH} using the same mannose-free crystallization conditions as done before to obtain the LD^{wt}-PD^{wt} tip structure (PDB 3JWN) [9]. While our attempts to crystallize LD^{A27V}-PD^{FocH} tip failed, the LD^{wt}-PD^{FocH} tip structure was solved at 2.8 Å resolution and deposited in the PDB with identifier 7SZO. As in the previously resolved isomorphous LD^{wt}-PD^{wt} tip structure, there are two LD^{A27V}-PD^{FocH} tip complexes in the asymmetric unit that can be superposed with a root-mean-square deviation (RMSD) of 1.33 Å for 926 Cα atoms common to each complex. Each complex contains a single copy of the FimH adhesin, one copy of the FimG subunit, two copies of the FimF subunit and one copy of the chaperone protein FimC that formed a complex with the proximal copy of FimF (FIG. 17A). The structural topology of the individual subunits was highly similar in LD^{wt}-PD^{wt} and LD^{wt}-PD^{FocH} tip structures, with RMSD values for the Cα atoms ranging from 0.14 Å for FimG to 0.93 Å for FimE. For the FimH adhesins (two copies of each in two crystal structures), the overall RMSD ranged from 0.28 Å to 0.55 Å. The relative orientations of the lectin and pilin domains are similar in all complexes (FIG. 17B). The difference in the primary structure of PD^{wt} and PD^{FocH} (FIG. 17C) did not affect significantly the secondary and tertiary structure of the pilin domain (RMSD 0.29-0.42 Å). The conformation of the lectin domain in LD^{wt}-PD^{FocH} and LD^{wt}-PD^{wt} are very similar (RMSD 0.27 Å; range 0.14-0.45 Å), but they differ significantly from the HAS (PDB 4XOC) and hybrid conformations (4XOE) of the lectin domains (RMSD 3.50 Å and 1.94 Å, respectively). Detailed comparison of the lectin domain conformation in the LD^{wt}-PD^{FocH} and LD^{wt}-PD^{wt} tip structures reveals identical secondary structures of the key conformationally variable regions. These include those involved in the interaction with pilin domain (see below for detailed analysis). As in LD^{wt}-PD^{wt}, the mannose-binding pocket in LD^{wt}-PD^{FocH} had the wide-open conformation. Thus, lectin domains in LD^{wt}-PD^{FocH} and LD^{wt}-PD^{wt} represent same allosteric conformers. i.e., are in the inactive, LAS conformation.

Thus, both the mAb824 and crystallography results indicate that, despite LD^{wt}-PD^{FocH} FimH exhibiting the HAS-like functional phenotype, the conformation of its lectin domain in the absence of mannose is primarily LAS, i.e., in the same state as LD^{wt}-PD^{wt} that exhibits a distinct LAS-like functional phenotype. In contrast, lectin domain in LD^{A27V}-PD^{FocH} appears to be sustained mostly in HAS according to the mAb824 studies, though no crystal structure could be obtained for this variant.

LD^{A27}-PD^{FocH} has Weakened Interdomain Interactions.

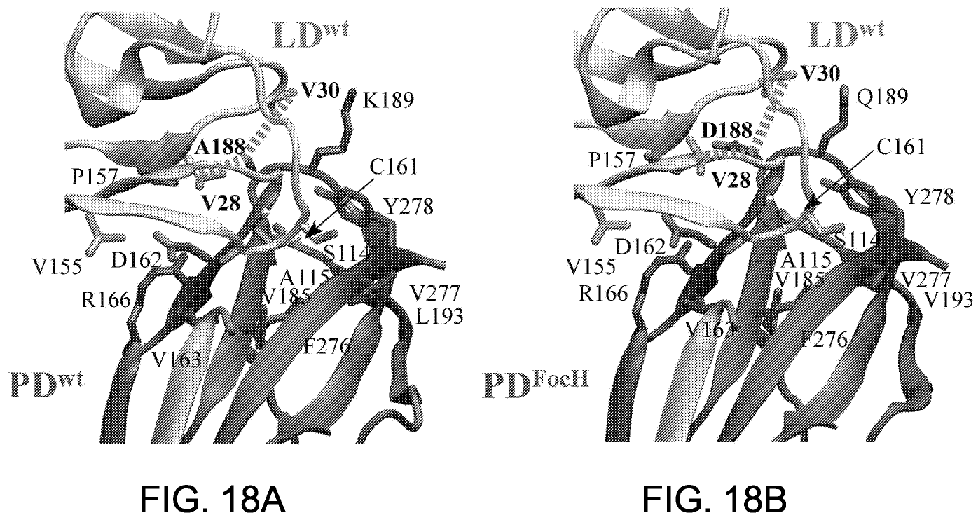

Given the high degree of similarity of the lectin domains in LD^{wt}-PD^{wt} and LD^{wt}-PD^{FocH}, we analyzed the inter-domain interface in the two crystal structures in more detail. In at least one of two complexes in the previously solved LD^{wt}-PD^{wt} structure, inter-domain contacts of the lectin domain with the pilin domain are observed between side chains of V28 with D162 and A188; V30 with A188 and K189; S114 with C161, L193, V277 and Y278; A115 with C161, V163, V185 and F276; V155 with R166; and P157 with D162 (FIG. 18A) [9]. In addition, there are hydrogen-bonding interactions between R166 side chain and the backbone carbonyl of A115, and between the carbonyl of S114 and the amide of C161. All of the inter-domain contacts observed in LD^{wt}-PD^{wt} are conserved in LD^{wt}-PD^{FocH} (FIG. 18B). The distances between side chains involved in inter-domain contacts differ by less than 0.5 Å and without a discernable pattern (Table 7).

TABLE 7
Inter-domain contact distances.

| residue | V28 | V30 | S114 | A115 | V155 | P157 |
|---|---|---|---|---|---|---|
| C161 (wt) | | | 5.7; 5.8 | 4.6; 4.0 | | |
| C161 (FocH) | | | 5.4; 5.4 | 4.4; 4.3 | | |
| D162 (wt) | 4.9; 5.0 | | | | | 5.4; 5.5 |
| D162 (FocH) | 4.9: 4.9 | | | | | 6.0; >6 |
| V163 (wt) | | | | 5.0; 5.1 | | |
| V163 (FocH) | | | | 4.8; 4.9 | | |
| R166 (wt) | | | | | 4.6; 4.7 | |
| R166 (FocH) | | | | | 4.7; 4.8 | |
| V185 (wt) | | | | 5.5; 5.5 | | |
| V185 (FocH) | | | | 5.4; 5.3 | | |
| A188 (wt) | 4.7; 4.9 | 4.7; 4.5 | | | | |
| D188 (FocH) | 5.2; 4.6 | 5.1; 5.2′ | | | | |
| K189 (wt) | | 4.5; 5.4 | | | | |
| Q189 (FocH) | | 4.7; 5.1 | | | | |
| L193 (wt) | | | 5.5; 5.7 | | | |
| V193 (FocH) | | | 5.3; 5.4 | | | |
| F276 (wt) | | | >6; >6 | 3.7; 3.8 | | |
| F276 (FocH) | | | 5.8; >6 | 4.0; 4.2 | | |
| V277 (wt) | | | 5.6; 5.6 | | | |
| V277 (FocH) | | | 5.8; 5.5 | | | |
| Y278 (wt) | | | 3.7; 3.7 | | | |
| Y278 (FocH) | | | 3.9; 4.1 | | | |

Within the residues 188-201 segment that contains sequence differences between PD^{wt} and PD^{FocH}, only two amino acids that differ between the two structures—A188D and K189Q—are involved in inter-domain interactions (L193 is unchanged between the two structures). As with the rest of interdomain interactions, the contact distances between V30-K189 vs. V30-Q189, V28-A188 vs. V28-D188 and V30-A188 vs. V30-D188 were not substantially different (Table 7). Despite A188 being hydrophobic and D188 being hydrophilic, the A188D replacement appears to be structurally tolerated. Indeed, the D188 side chain is rotated at its Cβ carbon, allowing it to interact with the same residues as the A188 Cβ carbon, and the carboxylate of D188 away from the interdomain interface towards solvent (FIG. 18B).

We used molecular dynamics (MD) simulations to determine the persistence of contacts made by replaced interface residues under force-free conditions. A side chain contact was defined to be persistent if the distance between the centers of geometry of two side chains did not exceed 6 Å during at least two-thirds of the frames. According to the simulations, the contacts of D188 with V28 and V30 in LD^{wt}-PD^{FocH} were less stable than those involving A188 in LD^{wt}-PD^{wt}. The interaction between D188 and V28 was particularly destabilized, remaining mostly broken in both simulations of LD^{wt}-PD^{FocH}, while the interaction between A188 and V28 was fully preserved in LD^{wt}-PD^{wt}. The effect of D188 relative to the native A188 is likely because the charged aspartate side chain does not support interactions with V28 and V30 as strongly as alanine at the same position. Indeed, while the LD and PD move slightly apart in simulations of both structures, the side chain of D188 but not A188 becomes hydrated, with a water molecule observed to reside between the carboxylate of D188 and the carbonyl of V28 in 60% of all simulation frames.

We also evaluated whether replacement of alanine in LD^{wt} to valine in LD^{A27V} could affect the conformations' stability. Analysis of the resolved structure of isolated LD^{wt} and LD^{A27V} (PDB codes 4XOC and 4XO8, respectively, both in the HAS conformation) reveals that A27 and V27 form a side chain contact with V155 located in a short β strand (AA 154-156). The distance between A27-V155 is only slightly larger than between V27-V155—5.8 Å and 5.4 Å, respectively. However, valine has a higher propensity for β strand formation than alanine and its side chain is oriented parallel to V155, providing a possible rationale for how V27 stabilizes the HAS conformation better than A27.

Taken together, the structural analyses reveal that there are only minor differences in the inter-domain bonds composition between LD^{wt}-PD^{wt} and LD^{wt}-PD^{FocH}, implying that PD^{FocH} is able to interact with the lectin domain similarly to PD^{wt} and, thus, exert the same allosteric effect to stabilize LD in LAS. From the MD analysis, however, it appears that rupture of the side chain contacts within the LD^{wt}-PD^{FocH} interface occurs more readily, likely due to the hydration of D188. Thus, although the inter-domain contacts are retained in LD^{wt}-PD^{FocH}, the interface contacts appear to be less stable. Finally, it is highly probable that the mutation A27V makes lectin domain more prone to stay in HAS. This, in turn, could lead to a more dynamic inter-domain configuration, especially in the PD^{FocH} background, potentially explaining why it is harder to resolve the crystal structure of the LD^{A27V}-PD^{FocH} tip complex.

FimH Variants Exhibit Different Dynamics of Conformational Switching.

The mAb21 binding experiments performed above (FIG. 16D-E) used a single 1 h incubation timepoint to measure the HAS prevalence, which does not allow assessment of the kinetics of LAS vs. HAS conformation populations. Thus, we measured mAb21 binding over a range of time points, starting with 10 sec minimal and up to 2 hours maximal incubation time.

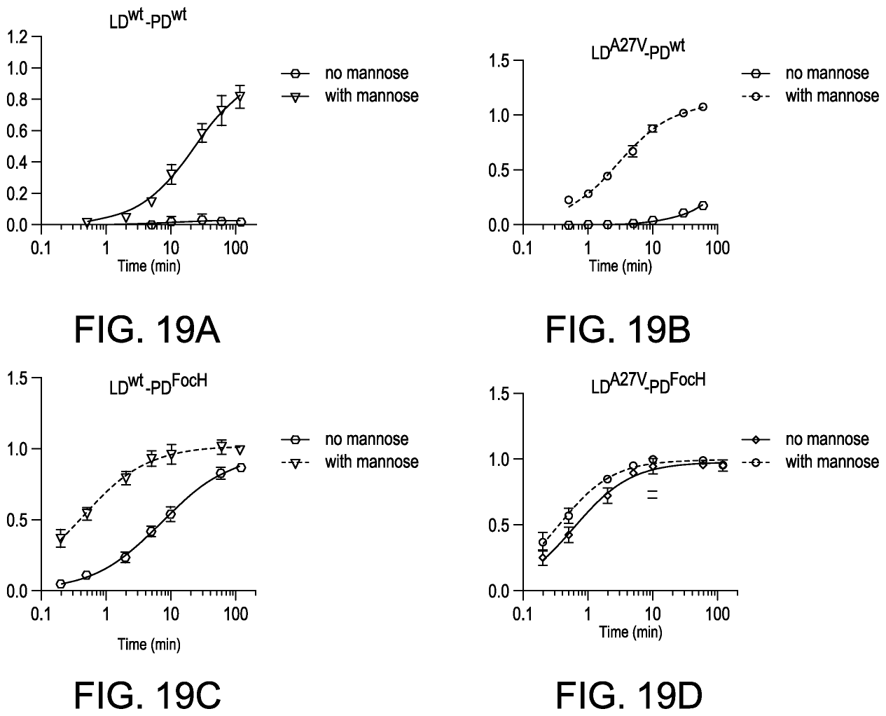

No reliably measurable binding of mAb21 was detected at any time point to LD^{wt}-PD^{wt} FimH (FIG. 19A). In the presence of soluble mannose, mAb21 binding to LD^{wt}-PD^{wt} steadily increased over time, nearly reaching a plateau level at the end of 2 h incubation, with 50% of the maximal optical density (OD₅₀) reached at ˜30 min. In the absence of mannose, the binding level of mAb21 to the LD^{A27V}-PD^{wt} variant was only marginal at the later time points (FIG. 19B). In the presence of mannose, mAb21 accumulation on LD^{A27V}-PD^{wt} was notably faster, with OD₅₀ reached in ˜3 min, i.e. much sooner than that with LD^{wt}-PD^{wt} (FIG. 19B).

As expected, mAb21 binding to LD^{wt}-PD^{FocH} was relatively fast even in the absence of mannose with OD₅₀ reached in ˜10 min (FIG. 19C). The binding rate was notably higher in the presence of mannose with the OD₅₀ reached in ˜30 sec. Notably, at the earliest time points, the accumulation level in the absence of mannose was only ˜10-15% of that level in the mannose presence. mAb21 binding rate to LD^{A27V}-PD^{FocH} in the absence of mannose was faster than to LD^{wt}-PD^{FocH}, with OD₅₀ reached in ˜40 sec, but in the presence of mannose, mAb21 accumulated even faster, with OD₅₀ reached in ˜20 sec (FIG. 19D). At the early time points, the antibody accumulation level in the absence of mannose for LD^{A27V}-PD^{FocH} was 70-75% of the binding in the presence of mannose.

Taken together, these results suggest that populations of LAS and HAS conformers are in a contrastingly different dynamic equilibrium in the different FimH variants. As expected, the equilibrium of the LAS and HAS conformers is altered in the presence of mannose. In both pilin domain backgrounds, LD^{A27V} is more prone than LD^{wt} to shift towards HAS, which appears to be the prevalent state in LD^{VA27V}-PD^{FocH} even in the absence of mannose. For LD^{wt}-PD^{FocH}, however, the results at early time points support the hypothesis that LAS is dominant in this FimH variant without mannose, while HAS been dominant when mannose is present.

The Differential Conformational Shift Dynamics Depends on the Rate of LD Deactivation.

By using the time course data of mAb21 binding, we sought next to estimate the kinetics and equilibrium constants for switching between LAS and HAS by calculating apparent rates of shift from LAS to HAS (i.e. the activation rate, k_{act}) and from HAS to LAS (deactivation rate, k_{deact}). It is expected that mAb21 binds to FimH at a rate constant (k_{mAb21}) that is the same for all variants in the absence or presence of mannose, because the structural differences do not directly impact the mAb21 epitope, and that mAb21 does not significantly detach from HAS in the time course of these assays [61]. Therefore, the rate of accumulation of mAb21 reflects the amount of FimH that is in the HAS conformation and not yet bound to mAB21. We therefore fit the data for each variant to the simple three-state model illustrated in FIG. 20A, as described in the Methods section.

Equilibrium constants (K_{eq}=k_{act}/k_{deact}) were obtained for all seven conditions in FIG. 19 that provided measurable mAb21 binding. As expected, the equilibrium constants were higher for variants that bound HRP better in FIG. 16C and were higher in the presence than in the absence of mannose for each variant (FIG. 20B).

The equilibrium constants provide a measurement of the percent of FimH in the HAS without mAb21. The two FimH variants with PD^{wt} sampled the HAS less than 0.1% of the time in the absence of mannose. Even in the presence of mannose, these variants had K^{eq}<1, so were mostly in the LAS. Also as expected, LD^{A27V}-PD^{FocH} displayed K_{eq}>1, so it was primarily in HAS both in the presence or absence of mannose. In contrast, the prevalence of HAS in LD^{wt}-PD^{FocH} shifted from only 13% in the absence of mannose to a majority in the presence of mannose. These findings are consistent with this variant binding HRP well (FIG. 16C) but not when its conformation was kinetically trapped by mAb824 in the absence of mannose (FIG. 16G), and why it crystalized in the LAS conformation. This modeling is therefore validated by the experimental findings.

For the five conditions with the highest K_{eq}, we also obtained reproducible kinetic constants, while for the other two conditions we obtained lower bounds on the rate constants. Remarkably, the activation rate constant k_{act} is similar for all of these conditions, with no substantial difference between FimH variants or the presence of mannose, while the deactivation rate constant k_{deact} was distinctly different (FIG. 20C). The deactivation was much faster in the naturally occurring FimH than in the recombinant variants and, for each variant, significantly decreased in the presence of mannose (FIG. 20C).

Thus, the model fitting affirms that, regardless of the presence of mannose, the variants with PD^{wt} are mostly in LAS and LD^{A27V}-PD^{FocH} is mostly in HAS. In contrast, in the absence of mannose, LD^{wt}-PD^{FocH} is mostly in LAS, while in the presence of mannose, it is mostly in HAS. Importantly, these differences between the FimH variants are only due to differences in the rate of deactivation of the lectin domain and not the rate of activation, which was similar for all variants and in the absence or presence of mannose.

Predominance of HAS is Associated with a Slow Rate of Mannose Binding.

In the experiments performed above, both recombinant FimH variants demonstrated strong binding of soluble HRP (FIG. 16C), but this was again determined by the single endpoint value obtained after 1 h of incubation, so does not address binding kinetics. Thus, we compared the rate of soluble HRP binding to LD^{wt}-PD^{FocH} and LD^{A27V}-PD^{FocH} fimbriae over time.

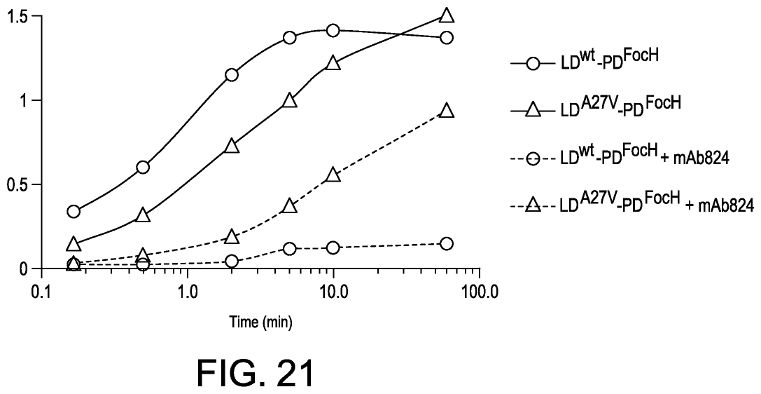

At the early time points, HRP accumulation on LD^{wt}-PD^{FocH} fimbriae increased faster than on LD^{A27V}-PD^{FocH} fimbriae (FIG. 21). This does not reflect a difference in the density of immobilized fimbriae, which are nearly identical in these experiments (FIG. 16F). We next determined the HRP binding rates to FimH pretreated with mAb824, which stabilizes the pre-existing conformation of FimH. As observed in FIG. 16G, binding of HRP to LD^{wt}-PD^{FocH} in complex with mAb824 saturated at very low levels, consistent with the lectin domain of this variant being predominantly in LAS. The rate of HRP binding to LD^{A27V}-PD^{FocH} in complex with mAb824, was dramatically reduced, supporting the notion that the activated conformation of FimH has a very low association rate (FIG. 21).

Taken together, these results indicate that lectin domain in HAS mediates mannose-specific binding at a significantly slower rate than the lectin domain in LAS, and that the locked LAS retains only marginal mannose-binding capability. In contrast, native LAS with the preserved ability to switch to HAS can bind quickly, demonstrating that even in the absence of tensile force, the allosteric conformational changes enable FimH to mediate both rapid and strong binding.

LAS is Required for Bacterial Adhesion Under Shear and Resistance to Inhibition.

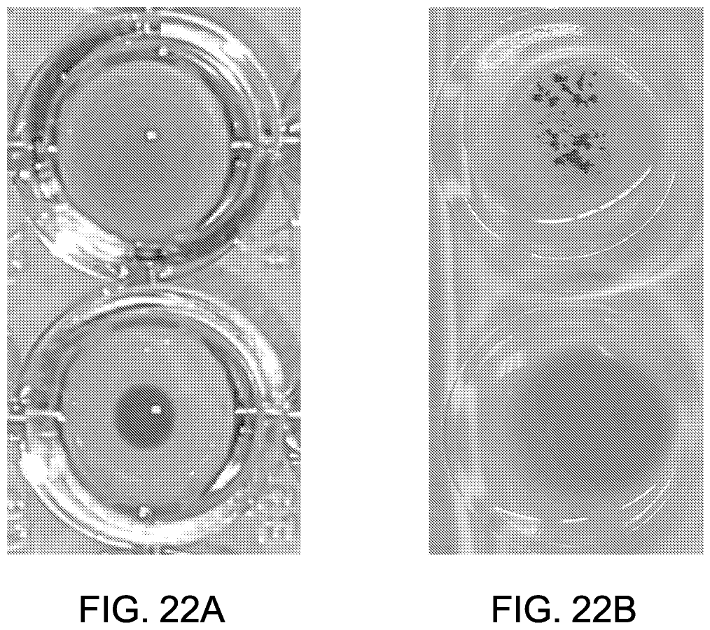

Finally, we compared cell binding ability of the FimH-expressing bacteria under static and dynamic conditions by using two classical set-ups with agglutination of guinea pig red blood cells (RBC)—the model cells for type 1 flmbriae-mediated bacterial adhesion. In the static assays, different dilutions of bacteria were combined with a suspension of RBC in U-shaped microtiter wells and allowed to settle without shaking. If bacteria adhere to RBC, a network of RBC (‘rosette’) is spread across the well, while if bacteria do not bind, RBC fall into a compact pellet (FIG. 22A). In the dynamic assay, bacteria and RBC were combined together on a slide and subjected to continuous back-and-forth rocking that creates fluidic shear stress. If bacteria bind to RBC, large compact clumps of RBCs are formed, while if bacteria do not bind, RBCs remain suspended (FIG. 22B).

As shown in previous studies [16], under static conditions bacteria expressing the LD^{wt}-PD^{wt} variant could not bind RBC at any concentration, while LD^{A27V}-PD^{wt} bacteria could form the agglutination rosettes but only at a relatively high concentration (low titer) of bacteria (Table 6). Under dynamic conditions, both LD^{wt}-PD^{wt} and LD^{A27V}-PD^{wt} bacteria were able to robustly aggregate RBC (Table 6).

TABLE 6
Red blood cell agglutination results. ND—not done; IC—inhibitory concentration.

| | Static conditions | | Dynamic conditions | |
|---|---|---|---|---|
| | Agglutination | Minimal IC | Aggregation | Minimal IC |
| FimH variant | titer | of mannose | level | of mannose |
| LD^{wt}-PD^{wt} | >1:1 | ND | +++ | .1% |
| LD^{A27V}-PD^{wt} | 1:2-1:4 | ND | ++++ | .1% |
| LD^{wt}-PD^{FocH} | 1:16 | .1% | ++++ | .004% |
| LD^{A27V}-PD^{FocH} | 1:8-1:16 | .004% | + | ND |

In contrast to the naturally occurring FimH, both recombinant variants were able to agglutinate RBC in the static conditions at relatively low concentrations (high titer) (Table 6). However, their RBC binding under the dynamic conditions were strikingly different from each other. While LD^{wt}-PD^{FocH} bacteria were robustly aggregating RBC similar to the bacteria with naturally occurring FimH, only a very weak aggregation was observed with the LD^{A27V}-PD^{FocH} bacteria (Table 6).

We next determined to what extent the RBC binding is susceptible to inhibition by soluble mannose. Agglutination of RBC under dynamic conditions by LD^{wt}-PD^{FocH} is significantly more susceptible to mannose than that by the naturally occurring variants (Table 6). At the same time, under the static conditions it is far less sensitive to inhibition than that mediated by LD^{A27V}-PD^{FocH}.

Thus, as shown previously, both naturally occurring variants of FimH were able to mediate robust RBC agglutination only under shear conditions, exhibiting a shear-dependent binding phenotype, while LD^{A27V}-PD^{FocH} is able to mediate robust binding only under static conditions, exhibiting a shear-inhibitable phenotype. In contrast, LD^{wt}-PD^{FocH} is able to mediate RBC binding under both static and shear conditions, i.e., exhibiting a novel phenotype of shear-independent adhesion. Adhesion mediated by the latter FimH variant is also far superior to the HAS-dominated LD^{A27V}-PD^{FocH} variant in the ability to resist inhibition by soluble mannose, though more easily inhibited than the naturally occurring variants.

### Discussion

While many structural and phenotypic properties of three FimH variants—the shear-dependent LD^{wt}-PD^{wt} and LD^{A27V}-PD^{wt} and the shear-inhibitable LD^{A27V}-PD^{FocH}—have been reported previously [2, 9, 16, 20]. LD^{wt}-PD^{FocH} has not been characterized in a great detail. This fact and the availability of a recently described monoclonal antibody with the conformation-trapping ability [60] prompted us to compare the four variants in various functional assays. Similar to LD^{A27V}-PD^{FocH}, LD^{wt}-PD^{FocH} demonstrated a strong static adhesion to yeast mannan and binding of soluble HRP (both are Mani model substrates) and was recognized well by the HAS-specific antibody mAb21 already in the absence of mannose. Surprisingly, however, the use of conformation-trapping mAb824 suggested that, while LD^{A27V}-PD^{FocH} is predominately in HAS, LD^{wt}-PD^{FocH} is predominantly in the LAS conformation like the low-binding naturally occurring variants. Indeed, the X-ray crystallography analysis of fimbrial tips in the absence of mannose revealed that LD^{wt}-PD^{FocH} structure is sustained in a typical LAS conformation. All contacts between the lectin and pilin domain in LD^{wt}-PD^{FocH} were virtually indistinguishable from those observed in LD^{wt}-PD^{wt} tip, in which the LAS conformation was originally resolved over a decade ago [9]. At the same time, however, MD simulations predicted that the contacts between the two domains are not as stable in LD^{wt}-PD^{FocH} as in LD^{wt}-PD^{wt}.

It had been suggested previously by MD simulations that, even in the native LD^{wt}-PD^{wt} variant, there is a certain level of ‘wobbliness’ between the domains, with about one-third of contacts being intermittent [62, 63]. Here, we show that two critical inter-domain side chain contacts that are stable with LD^{wt}-PD^{wt} are short-lived in LD^{wt}-PD^{FocH}. The key observation is a water molecule insertion into the interface via the carboxyl group of D188 in LD^{wt}-PD^{FocH}, which is not observed with A188 in LD^{wt}-PD^{wt} and results in rupture of hydrophobic interactions of D188 with the lectin domain residues V28 and V30. The critical importance of A188D in making the inter-domain interactions weaker had been supported by previous experimental studies [20, 59]. Interestingly, V28 was recently predicted being an allosteric ‘toggle’ switch residue [60]. Its side chain is buried within the inter-domain interface in LAS but becomes solvent exposed if the pilin domain is removed from the structure. In contrast, in HAS the V28 side chain is switched inside the lectin domain core. Such switching of the V28 orientation upon the domains' separation was also shown in a previously performed MD simulation [63]. Thus, sequestration of V28 into the lectin domain core when the inter-domain interface becomes exposed to water, could be a trigger for the core repacking cascade causing the LAS to HAS allosteric conversion.

Together, our results show that while LAS dominates in LD^{wt}-PD^{FocH} in the absence of mannose, HAS becomes predominant in this variant in the presence of mannose. This is in contrast to the naturally occurring FimH variants, where LAS remains dominant even in the presence of mannose. Thus, the HAS-like binding phenotype of LD^{wt}-PD^{FocH} is likely due to a sustained shift from LAS to HAS conformation upon mannose binding, even in the absence of shear. In naturally occurring FimH, this shift requires the presence of shear. In other words, if the inter-domain interaction is relatively unstable (as observed in LD^{wt}-PD^{FocH}), FimH activation occurs in a force-free, shear-independent manner. In contrast, if there is a stable interdomain interaction (as in the naturally occurring FimH), the sustained FimH activation requires tensile force, i.e., occurs in a shear-dependent manner. One can generalize, therefore, that in cases when bacterial adhesion is well manifested under no-shear conditions, it still can involve a switch in conformation of the adhesive domain, i.e. involve an allosteric mechanism. Under this mechanism, the adhesive domain is sustained in an ‘inactive’ conformation by interaction with the anchoring domain but readily converts to a stable ‘active’ conformation upon ligand binding, without a need for application of a shear-derived tensile force.

Quantitative analysis of the rates of mAb21 binding has also allowed us to estimate the rates of LAS to HAS conversion (i.e. the FimH activation rate) and vice versa—back from the HAS to LAS (the FimH deactivation rate). Surprisingly, for the experimental conditions where the activation rate could be measured, it was quite similar for different structural variants of FimH and, also, in the absence or presence of mannose. In other words, it appears that the conformational shift from LAS to HAS is an intrinsic property of the protein that occurs rather spontaneously, independently of the stability of inter-domain interaction or whether the ligand is attached to the binding pocket. In contrast, it was the deactivation rate that differed significantly under different conditions. The slower this rate is, such as in FimH variants with a less stable inter-domain interaction and when mannose is present, the more FimH favors the HAS conformation. However, further studies are needed to understand details of the intrinsic conformational dynamics of FimH.

Though quantitative fitting was not done for the time course of soluble HRP accumulation on the fimbrial carpet, it was clear that the binding to LD^{wt}-PD^{FocH} occurs at a significantly faster rate than that of LD^{A27V}-PD^{FocH}. Moreover, locking the LD^{A27V}-PD^{FocH} conformation, which is for the most part HAS, with mAb824 further decreased the rate of mannose-specific binding over 10-fold. Thus, we believe that the lower rate of mannose binding of LD^{A27V}-PD^{FocH} vs. LD^{wt}-PD^{FocH} is due to a heavy predominance in the former of HAS conformation. HAS has an intrinsically very slow on-rate of ligand binding [2, 60], likely due to a narrow configuration of the binding pocket. Accordingly, the faster rate of binding of LD^{wt}-PD^{FocH} over LD^{A27V}-PD^{FocH} is due to the fact that the former variant has a higher proportion of LAS, which is able to convert in a sustainable manner to HAS even under equilibrium conditions. Furthermore, though LAS is a minor conformation in LD^{A27V}-PD^{FocH}, its activity is sufficient to provide a faster mannose binding than that of LD^{A27V}-PD^{FocH} in complex with mAb824.

It has previously been hypothesized that FimH variants favoring the LAS state will bind through ‘induced fit’ model, in which binding to ligand induces an activating conformational change [12]. As described above, however, our data contradict the hypothesis that the FimH binding mode involves the induced fit, because the rate of conversion of LAS to HAS is not increased by the mannose. In the same study, it was also hypothesized that some naturally occurring FimH variants that favor the HAS state will bind via a “conformational selection” pathway in which ligand selects for binding the combining pocket that is already in the ‘active’ conformation. However, our data presented here and, also, in previous studies indicate that mannose binding to HAS might be happening too slow to be physiologically relevant [2, 60]. We believe that, instead of following the induced fit or conformational selection models, FimH binding in natural environments is best described by a “kinetic selection” model in which ligand binds preferentially to LAS due to the fast ligand-binding kinetics associated with an open pocket, followed by conversion to HAS to avoid detachment. Originally, this model was proposed to explain how native FimH variants bind well in flow where tensile force induces the switch from LAS to HAS after ligand binds [2]. However, this study illustrates how kinetic selection can provide rapid and robust binding in static conditions as well, where the adhesin switches from LAS to HAS and remains in mannose-bound HAS, with the domains separation sustained even in the absence of force. We propose that, because the molecular mechanism of FimH activation remains essentially the same with or without the force, the LD^{wt}-PD^{FocH}-like adhesion could also be defined as a form of the allosteric catch bond. In this variant, the allosteric conformational change does not act as a regulatory mechanism that limits binding to high shear conditions. Instead, its allostery enables more effective binding in all conditions, and so provides a fundamentally new function for allostery.

It had been described previously that bacteria expressing naturally occurring FimH do not bind well to RBC under static conditions but readily aggregate them under rocking conditions, in agreement with their shear-dependent cell adhesion phenotype [16]. We also reported previously the ability of LD^{A27V}-PD^{FocH} to bind well to RBC under static conditions but weakly when rocked, demonstrating shear-inhibitable phenotype and reflecting the inability of HAS to form adhesive bonds fast enough under dynamic conditions [2, 60]. In contrast to the previously described FimH, LD^{wt}-PD^{FocH} demonstrated a novel shear-independent adhesion phenotype with strong binding to RBC under both static and shear conditions. One can attribute this to the combination of two properties in LD^{wt}-PD^{FocH}: the predominance of the inactive state needed for fast binding under rocking and the LD^{wt}-PD^{FocH} ability to shift to a stable HAS even in the absence of shear. One can also reason that the shear-independent adhesive phenotype is superior to the shear-inhibitable phenotype for enabling the bacterial attachment to target cells under a broader range of shear conditions. Moreover, because host compartments are often bathed with free-floating mannosylated glycoproteins that can act as adhesion inhibitors, the LD^{wt}-PD^{FocH}-like adhesins could have another advantage over the LD^{A27V}-PD^{FocH}-like adhesins by being more resistant to inhibition by mannose. This could be explained by a high likelihood that, upon the conversion to HAS, LD^{wt}-PD^{FocH} is still more readily reverted to LAS than LD^{A27V}-PD^{FocH}, effectively allowing the FimH adhesin to shed off the attached inhibitor.

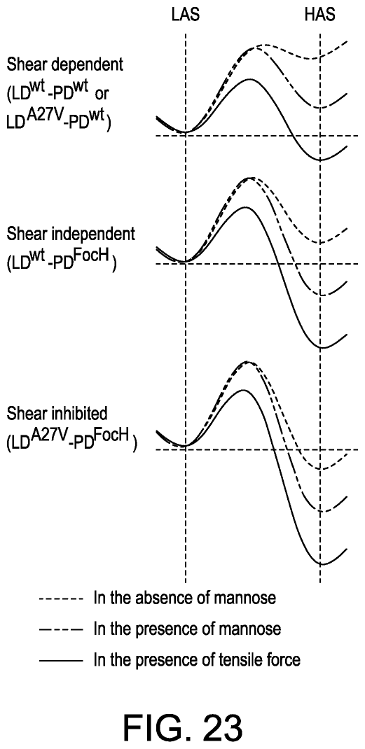

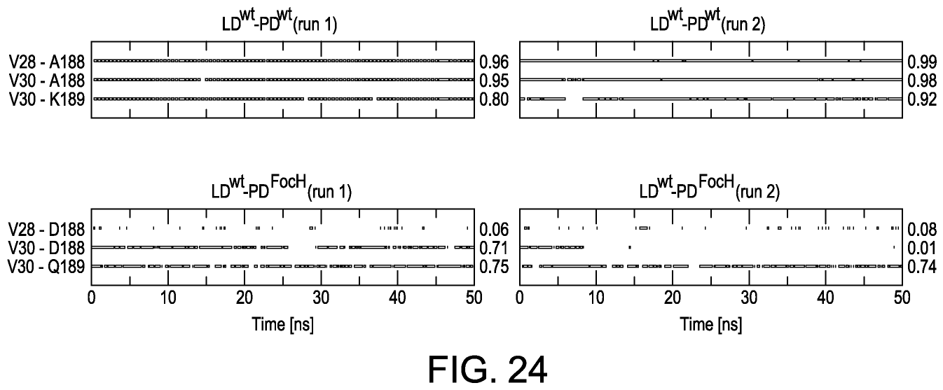

The differences in the conformational dynamics and binding modes between different FimH variants can be described in terms of free energy landscapes, where lower free energy indicates a more stable conformational state (FIG. 23). Both mannose and tensile force favor HAS over LAS, but this influence has a different impact on the different variants. For the shear-dependent variants, the energy of HAS is so much higher than LAS that the latter dominates even with mannose and HAS requires tensile force to become lower in energy. The allosteric catch bond property of FimH therefore causes the dramatic shear-dependence of these variants. For the shear-independent variants, LAS is lower in energy and thus dominant in the absence of mannose, while HAS been lower in energy and thus dominates in the presence of mannose. This allows the shear-independent variants to benefit from both the fast mannose-binding rate of LAS, and the long lifetimes of HAS. Thus, the allosteric catch bond property of FimH contributes to binding function even in static conditions that provide no tensile force. At the same time, for the shear-inhibitable variants, HAS is lower in energy than LAS even without mannose and thus dominates in all conditions. This FimH does not benefit from the fast mannose-binding rate of LAS and, thus, the allosteric catch bond behavior.

Finally, the shear-independent adhesion is more resistant to inhibition by soluble mannose than the shear-inhibitable adhesion, but is more sensitive than naturally occurring FimH with shear-dependent phenotype. This is expected as HAS is unstable in the shear-dependent variants even in the presence of soluble mannose. This also might explain why FimH variants with shear-dependent phenotype remain predominant in nature [52, 55]. However, because the shear-independent adhesive mode can accomplish the bacterial attachment under lesser shear, it could be advantageous over the shear-dependent adhesion in some low-shear host compartments, especially where the concentration of soluble ligand-like compounds is relatively low. This could be the case for FimH carried by E. coli found in patients with UTI that tend to carry ‘FimH-activating’ mutations close to the inter-domain interface in both LD and PD [53, 55, 56, 59]. Those mutations result in a shear-independent-like FimH phenotype with a higher-level adhesion under no or low shear, often more pronounced than the A27V mutation but generally not as pronounced as mediated by LD^{wt}-PD^{FocH} [55, 59]. One way or another, those mutations likely facilitate colonization of urinary tract compartments with both low and high flow conditions, like the bladder lumen or renal pelvis and urethra, respectively, and also a relatively low urine concentration of mannosylated glycoproteins like Tamm-Horsfall protein. [64, 65, 66]. At the same time, the shear-inhibitable phenotype, like in LD^{A27V}-PD^{FocH}, would be detrimental in the urinary tract because of its extremely high sensitivity to blockage even by minute amounts of inhibitors in urine. These observations could explain why an engineered variant that was obtained in the HAS state even without mannose was deficient relative to the natural UTI isolate variant in an animal model of UTI, even though it was more effective in infecting uroepithelial cells in static conditions in vitro [12]. Therefore, the variants predominantly in HAS conformation would not benefit from the allosteric catch bond behavior and would thus exhibit a slower on-rate, be more susceptible to inhibition, and be shear-inhibited.

### REFERENCES

1. Ofek I, et al. Bacterial Adhesion to Animal Cells and Tissues. ASM Press 2003, Washington, DC; ISBN 1-55581-263-5.
2. Yakovenko O, et al. (2015) Proc Natl Acad Sci USA; 112(32):9884-9.
3. Sokurenko E V, et al. (2008) Cell Host Microbe. October 16; 4(4):314-23.
4. Arnaout M A, et al. (2005) Annu Rev Cell Dev Biol.: 21: 381-410.
5. Alsteens D, et al. (2017) Nat Nanotechnol. February; 12(2):177-183.
6. Hospenthal M K, & Waksman G. (2019) Microbiol Spectr. January; 7(1).
7. Abraham S N, et al. (1988) Nature. December 15; 336(6200):682-4.
8. Choudhury D, et al. (1999) Science. August 13; 285(5430):1061-6.
9. Le Trong I, et al. (2010) Cell. May 14; 141(4):645-55.
10. Sauer M M, et al. (2016) Nat Commun. March 7; 7:10738.
11. Sauer M M, et al. (2019) J Am Chem Soc. January 16; 141(2):936-944.
12. Kalas V, et al. (2017) Sci Adv. February 10; 3(2):e1601944.
13. Rodriguez V B, et al. (2013) J Biol Chem. August 16; 288(33):24128-39.
14. Thomas W E, et al. (2008) Annu Rev Biophys.; 37:399-416.
15. Zihlmann P, et al. (2018) Chemistry. September 3; 24(49):13049-13057.
16. Thomas W E, et al. (2002) Cell. June 28:109(7):913-23.
17. Thomas W E, et al. (2004) Mol Microbiol. September; 53(5):1545-57.
18. Yakovenko O. et al. (2008) J Biol Chem. April 25; 283(17):11596-605.
19. Forero M, et al. (2006) PLoS Biol. September; 4(9):e298.
20. Aprikian P, et al. (2007) J Biol Chem. August 10; 282(32):23437-46.
21. Thomas W. (2006) J Cell Biol. September 25; 174(7):911-3.
22. Konstantopoulos K, et al. (2003) Curr Biol. August 5; 13(15):R611-3.
23. Dembo M, et al. (1988) Proc R Soc Lond B Biol Sci. June 22; 234(1274):55-83.
24. Thomas W. (2008) Annu Rev Biomed Eng.; 10:39-57.
25. Mathelié-Guinlet M, et al. (2021) Trends Microbiol. April; 29(4):286-288.
26. Kong F., et al. (2009) J. Cell Biol.; 185:1275-1284.
27. Chen W, et al. (2010) J Biol Chem. November 12; 285(46):35967-78.
28. Fiore V F, et al. (2014) Nat Commun. September 12; 5:4886.
29. Rakshit S, et al. (2012) Proc Natl Acad Sci USA. November 13; 109(46):18815-20.
30. Manibog K, et al. (2014) Nat Commun. June 2; 5:3941.
31. Sarangapani K K, et al. (2004) J Biol Chem. January 16; 279(3):2291-8.
32. Marshall B T, et al. (2003) Nature. May 8; 423(6936):190-3.
33. Snook J H, & Guilford W H. (2010) Cell Mol Bioeng. June 1; 3(2):128-138.
34. Das D K, et al. (2015) Proc Natl Acad Sci USA. February 3; 112(5):1517-22.
35. Sibener L V, et al. (2018) Cell. July 26; 174(3):672-687.e27.
36. Guo W H, et al. (2006) Biophys J. March 15; 90(6):2213-20.
37. Lee C Y, et al. (2013) Proc NaH Acad Sci USA. March 26; 110(13):5022-7.
38. Huang D L, et al. (2017) Science. August 18; 357(6352):703-706.
39. Litvinov R I, et al. (2013) J Biol Chem. November 15; 288(46):33060-70.
40. Stahlhut S G, et al. (2009) J Bacteriol. November; 191(21):6592-601.
41. Tchesnokova V, et al. (2010) Mol Microbiol. April; 76(2):489-502.
42. Ding A M, et al. (2010) Appl Environ Microbiol. February; 76(4):1294-7.
43. Kisiela D I, et al. (2011) J Biol Chem. November 4; 286(44):38136-38147.
44. Ebady R, et al. (2016) Cell Rep. September 6; 16(10):2593-2604.
45. Yakovenko O, et al. (2018) Infect Immun. May 22; 86(6):e00160-18.
46. Mathelié-Guinlet M, et al. (2020) Nat Commun. October 27; 11(1):5431.
47. Liu Z, et al. (2020) Nat Commun. August 28; 11(1):4321.
48. Becke T D, et al. (2019) ACS Nano. June 25; 13(6):7155-7165.
49. Anderson B N, et al. (2007) J Bacteriol. March; 189(5):1794-802.
50. Nilsson L M, et al. (2006) Appl Environ Microbiol. April; 72(4):3005-10.
51. Chan C X, & Lipke P N. (2014) Eukaryot Cell. September; 13(9):1136-42.
52. Sokurenko E V, et al. (1998) Proc Natl Acad Sci USA. July 21; 95(15):8922-6.
53. Weissman S J, et al. (2007) Infect Immun. July; 75(7):3548-55.
54. Rabbani S, et al. (2018) J Biol Chem. February 2; 293(5):1835-1849.
55. Sokurenko E V, et al. (2004) Mol Biol Evol. July; 21(7):1373-83.
56. Chattopadhyay S, et al. (2007) J Mol Evol. February; 64(2):204-14.
57. Beskhlebnaya V A, et al. (2006) Bull Exp Biol Med. March; 141(3):339-42.
58. Sokurenko E V, et al. (2001) Mol Microbiol. August; 41(3):675-86.
59. Tchesnokova V, et al. (2008) J Biol Chem. March 21; 283(12):7823-33.
60. Kisiela D I, et al. (2021) PLoS Pathog. April 7; 17(4):e1009440. (See also Example 1.)
61. Tchesnokova V, et al. (2011) Infect Immun. October; 79(10):3895-904.
62. Aprikian P, et al. (2011) PLoS Biol. May; 9(5):e1000617.
63. Interlandi G, & Thomas W E. (2016) Proteins. July; 84(7):990-1008.
64. Pak J, et al. (2001) J Biol Chem. March 30; 276(13):9924-30.
65. Cavallone D, et al. (2004) J Biol Chem. January 2; 279(1):216-22.
66. Bates J M, et al. (2004) Kidney Int. March; 65(3):791-7.
67. Sokurenko E V, et al. (1994) J Bacteriol. February; 176(3):748-55.
68. Sokurenko E V, et al. (1997) J Biol Chem. July 11; 272(28):17880-6.
69. Kisiela D I, et al. (2015) PLoS Pathog. May 14; 11(5):e1004857.
70. Otwinowski Z, & Minor W. (1997) Methods Enzymol.; 276:307-26.
71. Murshudov G N, et al. (1997) Acta Crystallogr D Biol Crystallog. May 1; 53(Pt 3):240-55.
72. Collaborative Computational Project, Number 4. The CCP4 suite: programs for protein crystallography. (1994) Acta Crystallogr Biol Crystallogr. September 1; 50(Pt 5):760-3.
73. Brünger A T. (1992) Nature. January 30; 355(6359):472-5.
74. Read, R. J. (1986) Acta Crystallographica Section A. Volume 42, Issue 3, May, Pages 140-149.
75. McRee D E. (1999) J Struct Biol. April-May; 125(2-3):156-65.
76. Emsley P. & Cowtan K. (2004) Acta Crystallogr D Biol Crystallogr. December; 60(Pt 12 Pt 1):2126-32.
77. Kalé L, et al. (1999) Journal of Computational Physics. Volume 151, Issue 1, Pages 283-312.
78. MacKerell, A D, et al. (1998) Journal of Physical Chemistry B, v. 102, pp. 3586-3616.
79. MacKerell, A D, et al. (2004) Journal of the American Chemical Society (JACS), v 126, n 3, pp. 698-699.
80. Mackerell, A D, et al. (2004) Journal of Computational Chemistry, v 25, n 11, pp. 1400-1415.
81. Darden, T. et al. (1993) J. Chem. Phys, v 98, pp 10089-10092.
82. Schneider, T. & Stoll, E. (1978) Physical Review B, v 17, n 3. pp. 302-1322.
83. Feller, S. E. et al. (1995) Journal of chemical physics; v 103, n 11, pp. 4613-4621.
84. Merkel R, et al. (1999) Nature. January 7; 397(6714):50-3.

### Example 8: Increasing Residue Hydrophobicity to Keep it in the Core

In addition to mutating to a charged residue to keep it in the solvent, one can increase the residue hydrophobicity to keep it in the core. We replaced an amphipathic amino acid tyrosine in position 64 of FimH to a more hydrophobic phenylalanine that is predicted to stabilize residue 64 inside the core. This is the orientation in low-affinity conformation and makes it less energetically favorable to switch to the solvent. The latter is the orientation in the high affinity conformation. As result, the low-affinity conformation became significantly more stable.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An allosteric cell attachment protein stabilized in an ‘active’ (high-affinity) or ‘inactive’ (low-affinity) conformation comprising a substitution of a hydrophobic amino acid residue with a charged amino acid.

2. The protein of claim 1, which is a bacterial adhesin.

3. The protein of claim 1, wherein the substitution stabilizes the protein in an active conformation.

4. The protein of claim 1, wherein the substitution stabilizes the protein in an inactive conformation.

5. The protein of claim 1, wherein the hydrophobic amino acid residue is alanine, tyrosine, isoleucine, leucine, methionine, phenylalanine, or valine.

6. The protein of claim 1, wherein the substitution comprises a charged glutamic acid or lysine substituted for a leucine, tyrosine, phenylalanine, and/or a valine.

7. The protein of claim 6, wherein the protein is Escherichia coli (E. coli) fimbrial adhesin FimH (SEQ ID NO: 1, 2, or 4), and the substitution is at L34, V35, and/or Y64.

8. The protein of claim 6, wherein the protein is E. coli fimbrial adhesin FmIH (SEQ ID NO: 3), and the substitution is at L32, V33, and/or F63.

9. An immunogenic composition comprising the protein of claim 1, and an adjuvant.

10. A method of producing antibodies directed against a cell adhesion protein comprising:
(a) administering the composition of claim 9 to a subject;
(b) obtaining a sample of blood from the subject; and
(c) isolating antibodies from the sample of blood,
wherein the composition of claim 9 comprises a protein stabilized in an inactive conformation.

11. The method of claim 10, wherein the antibodies are neutralizing antibodies.

12. A method of stabilizing an allosteric cell adhesion protein in an active or inactive conformation, the method comprising substituting a hydrophobic amino acid residue with a charged amino acid.

13. The method of claim 12, wherein the protein is a bacterial adhesin.

14. The method of claim 12, wherein the hydrophobic amino acid residue is alanine, isoleucine, leucine, methionine, phenylalanine, tyrosine, or valine.

15. The method of claim 12, wherein the substitution stabilizes the protein in an inactive conformation.

16. The method of claim 12, wherein the substitution stabilizes the protein in an active conformation.

17. The method of claim 12, wherein the substitution comprises a charged amino acid substituted for an aliphatic or aromatic amino acid.

18. The method of claim 17, wherein the protein is Escherichia coli (E. coli) fimbrial adhesin FimH (SEQ ID NO: 1, 2, or 4), and the substitution comprises L34, V35, or Y64.

19. The method of claim 17, wherein the protein is E. coli fimbrial adhesin FmIH (SEQ ID NO: 3), and the substitution comprises L32, V33, and F63.

20. The method of claim 17, wherein the charged amino acid is a charged glutamic acid or a charged lysine.
